(19) United States
(12) Patent Application Publication
(10) Pub. No.: US 2013/0216495 A1
(43) Pub. Date: Aug. 22, 2013

(54) PHARMACEUTICAL COMPOSITION COMPRISING CD34+ CELLS

(71) Applicants: BAXTER INTERNATIONAL INC., (US); BAXTER HEALTHCARE SA, (US)

(72) Inventors: Delara Motlagh, Barrington, IL (US); Amy Cohen, Grayslake, IL (US); Lora Palmer, Algonquin, IL (US); David L. Amrani, Glendale, WI (US)

(73) Assignees: BAXTER HEALTHCARE SA, Glattpark (Opfikon) (CH); BAXTER INTERNATIONAL INC., Deerfield, IL (US)

(21) Appl. No.: 13/773,390

(22) Filed: Feb. 21, 2013

Related U.S. Application Data
(60) Provisional application No. 61/601,326, filed on Feb. 21, 2012.

(51) Int. Cl. A61K 35/14; A61K 35/12

(52) U.S. Cl. CPC A61K 35/14; A61K 35/12
USPC 424/85.1; 424/93.7

(57) ABSTRACT

The invention relates to the field of treatment of ischemic conditions and diseases using a cell population comprising CD34+ cells isolated from peripheral blood of a subject. The invention provides a pharmaceutical composition comprising (i) a cell population comprising CD34+ cells, (ii) a plasma protein, and (iii) an isotonic solution comprising at least one salt, said isotonic solution comprising acetate, gluconate, or both acetate and gluconate. Methods of treating tissue damaged by ischemia in a subject and methods of treating a medical condition, wherein the pharmaceutical composition of the invention is administered, are further provided herein. Also, methods of promoting mobilization of CD34+ cells from bone marrow into peripheral blood are provided herein.

### CROSS-REFERENCE TO RELATED APPLICATIONS

This application claim priority to U.S. Provisional Application No. 61/601,326, filed Feb. 21, 2012, the contents of which are incorporated herein by reference in its entirety.

### FIELD OF THE INVENTION

The present invention relates to the field of treatment of ischemic conditions and diseases using a cell population comprising CD34⁺ cells isolated from peripheral blood of a subject.

### BACKGROUND

It has been a goal of scientists and doctors to use stem cells to treat diseases by administering these cells to sites of disease, where it is hoped that the cells will regenerate or repair the tissue. All mammalian cells require a consistent source of oxygen to allow them to function normally. When their access to oxygen is interrupted, cell damage and death can quickly result. Certain cell types, including, but not limited to, muscle cells and neurons, are particularly vulnerable to ischemic injury in connection with myocardial infarction and stroke. Despite recent advances in treating ischemic injuries, stroke and myocardial infarction continue to kill or disable vast numbers of people each year. Accordingly, improved methods of treating tissue injury, particularly ischemic injuries associated with stroke and myocardial infarction, are needed.

### SUMMARY OF THE INVENTION

The invention provides pharmaceutical compositions comprising CD34-positive (CD34+) cells useful for administration to a subject in need thereof. In exemplary aspects, the pharmaceutical compositions are useful in methods of repairing tissue damaged by ischemia in a subject or in methods of treating a medical condition, including, but not limited to, chronic myocardial ischemia, critical limb ischemia, peripheral artery disease, Buerger's disease, ischemic heart disease, ischemic colitis, mesenteric ischemia, brain ischemia, cerebral ischemia, acute limb ischemia, and renal ischemia, since the CD34+ cells are stem cells (e.g., but without limitation to pluripotent stem cells, totipotent stem cells, or multipotent stem cells).

The pharmaceutical compositions of the invention are suitable for both short term storage of the CD34+ cells at non-freezing temperatures, as well as for direct administration to the subject. The pharmaceutical compositions suitably provide a stable storage environment for the CD34+ cells, such that the number of viable and functional cells in the pharmaceutical composition is maximized during the course of storage and/or transportation of the pharmaceutical composition that occurs prior to administration of the pharmaceutical composition. In exemplary aspects, the pharmaceutical compositions are suitable for storage of CD34+ cells for a time period of less than 5 days (e.g., but without limitation to 4, 3, or 2 days) at a temperature between 1 and 30 degrees Celsius (e.g., but without limitation to 2 to 8 degrees Celsius) and for direct administration to the subject.

The pharmaceutical compositions of the invention thus avoid the need for two separate solutions—one for storage and one for administration to the subject. Accordingly, a transferring step, in which the CD34+ cells are transferred from a storage solution to an administration solution (e.g., but without limitation to, a pharmaceutically acceptable carrier, excipient, or diluent) is not needed when preparing the pharmaceutical compositions of the invention.

In exemplary aspects, the pharmaceutical composition comprises (i) a cell population comprising CD34⁺ cells, (ii) a plasma protein and (iii) an isotonic solution comprising at least one salt. The pharmaceutical composition is optionally formulated for intravenous administration. In exemplary aspects, the pharmaceutical composition comprises (i) a cell population comprising CD34+ cells, (ii) a plasma protein, and (iii) an isotonic solution comprising at least one salt, said isotonic solution comprising a preservative or a stabilizing agent. In exemplary aspects, the pharmaceutical composition comprises (i) a cell population comprising CD34+ cells, (ii) a plasma protein, and (iii) an isotonic solution comprising at least one salt, said isotonic solution comprising a bicarbonate precursor. In exemplary aspects, the pharmaceutical composition comprises (i) a cell population comprising CD34+ cells, (ii) a plasma protein, and (iii) an isotonic solution comprising at least one salt, said isotonic solution comprising acetate, gluconate, or both acetate and gluconate.

In some embodiments, the cell population comprises a heterogenous cell population of which at least 1% (e.g., but without limitation to, at least 5%, at least 10%, at least 15%, at least 20%, at least 25%, at least 30%, at least 35%, at least 40%, at least 45%, at least 50%, at least 55%, at least 60%, at least 65%, at least 70% at least 75%, at least 80%, at least 85%, at least 90%, at least 95%, at least 96%, at least 97%, at least 98%, or at least 99%) of the cells of the cell population are CD34+ cells. The cells of the cell population optionally also express one or more cell markers including, but not limited to, CXCR4, c-kit (CD117), FLK-1 (VEGFR-1), Tie-2 and KDR (VEGFR-2), CD133, CD45, CD14, CD64, CD61, CD141, CD33, CD38, CD31, CD105, CD146, CD144, CD73, CD99, CD29 and CD90. In certain aspects, the cell population comprises a subset of cells that co-express CXCR4⁺/CD34⁺. For example, in some embodiments, at least 0.1% of the cells in the cell population are CXCR4⁺/CD34⁺ cells. Cell populations comprising at least 0.5%, at least 1%, at least 2%, at least 3%, at least 4%, at least 5%, at least 6%, at least 7%, at least 8%, at least 9%, at least 10%, at least 15%, at least 20% or more CXCR4⁺/CD34⁺ cells are also contemplated.

The cell population in the pharmaceutical composition optionally comprises at least 70%, at least 75%, at least 80%, at least 85%, at least 90%, at least 95% or more viable cells in the cell population. In some embodiments, at least 70%, at least 75%, at least 80%, at least 85%, at least 90%, at least 95% or more of the cells of the cell population are viable cells after storage in the pharmaceutical composition for a period of time from about 1 hour to about 5 days.

In certain aspects, at least 1%, at least 5%, at least 10%, at least 15%, at least 20%, at least 25% or more of the cells in the cell population have a migration index which is greater than that of a control, as determined by, for example, but without limitation to, a chemokine gradient migration assay after storing the pharmaceutical composition from about 1 hour to about 5 days at a temperature of about 1° C. to about 30° C.

The isotonic solution of the pharmaceutical composition optionally comprises one or more of sodium, potassium, magnesium, chloride, acetate and gluconate at various concentrations. In some embodiments, the isotonic solution comprises about 100 mEq to about 180 mEq sodium and optionally further comprises one or more of about 1 mEq to about 9 mEq potassium, about 0.5 mEq to about 5.5 mEq magnesium, about 70 mEq to about 120 mEq chloride, about 10 mEq to about 40 mEq acetate, and about 10 mEq to about 40 mEq gluconate. In certain aspects, the isotonic solution has a pH or about 6.5 to about 7.8 and an osmolality of about 240 mOsmol/L to about 350 mOsmol/L. In exemplary aspects, the isotonic solution is calcium-free. The pharmaceutical composition is optionally free of dextrose.

In exemplary aspects, the total protein concentration attributed by the plasma protein(s) of the pharmaceutical composition is less than that of human plasma (e.g., but without limitation, less than 60 g/L). In exemplary aspects, the pharmaceutical composition comprises no more than five different plasma proteins. In exemplary aspects, the pharmaceutical composition comprises no more than one plasma protein. In certain aspects, the plasma protein of the pharmaceutical composition is albumin (e.g., but without limitation to, human serum albumin). The pharmaceutical composition, in some embodiments, comprises human serum albumin in an amount of at least or about 0.5% (w/v). In some embodiments, the pharmaceutical composition comprises human serum albumin in an amount ranging from about 1% (w/v) to about 10% (w/v) or from about 3% to about 7%.

In exemplary embodiments, the pharmaceutical composition comprises serum or plasma and the total protein concentration attributed by the plasma protein(s) of the pharmaceutical composition is less than 60 g/L. In some embodiments, the serum or plasma is present in the pharmaceutical composition at a concentration less than 20% (v/v). In exemplary aspects, the pharmaceutical composition comprises an isotonic solution free of calcium and the only source of calcium in the pharmaceutical composition is the calcium present in the plasma or serum.

In certain aspects, the pharmaceutical composition is formulated for intravenous administration. In some embodiments, the pharmaceutical composition is packaged in a ready to use and/or non-reusable container, such as a syringe, vial or bag. The ready to use and/or non-reusable container optionally comprises a unit dose of the pharmaceutical composition described herein.

Also described herein is a method of repairing tissue damaged by ischemia in a subject comprising administering a pharmaceutical composition described herein to the subject in an amount effect to repair damaged tissue in the subject. Also, a method of treating a medical condition (including, but not limited to, myocardial ischemia, critical limb ischemia, peripheral artery disease, Berger's disease, ischemic heart disease, ischemic colitis, mesenteric ischemia, brain ischemia, cerebral ischemia, or acute limb ischemia) in a subject in need thereof is provided. In exemplary aspects, the method comprises administering a pharmaceutical composition described herein to the subject in an amount effective to treat the medical condition.

Methods of promoting mobilization of CD34⁺ cells from bone marrow into peripheral blood in a subject are provided. Such methods comprise administering to the subject granulocyte colony stimulating factor (G-CSF) at a total administered dose of less than 50 μg/kg, about 40 μg/kg or less, about 30 μg/kg or less or about 25 μg/kg or less. In certain embodiments, the total administered dose of G-CSF is administered within 7 days (e.g., but without limitation to, 7 days, 6 days, 5 days, 4 days, 3 days, 2 days). In other embodiments, the total administered dose of G-CSF is administered within 5 days. In yet other exemplary embodiments, the total administered dose of G-CSF is administered over a course of about 4 days. Without being bound to any particular theory, such methods of promoting CD34+ cell mobilization result in sufficient numbers of CD34+ cells in the peripheral blood for subsequent collection and administration for treatment, yet reduces the potential for pain and discomfort to the subject and reduces the time during which mobilization occurs.

In some embodiments, the G-CSF is administered to the subject at a dose between 2.5 μg/kg/day to about 7.0 μg/kg/day or at a dose between about 4 μg/kg/day and 6 μg/kg/day. The G-CSF is optionally administered to the subject at a dose between 4.8 μg/kg/day and 5.2 μg/kg/day for 5 days. In exemplary aspects, the G-CSF is administered to the subject at a dose between 4.8 μg/kg/day and 5.2 μg/kg/day for not more than 4 or 5 days.

Methods of obtaining CD34⁺ cells from a subject are also provided. Such methods comprise the step of promoting mobilization of CD34⁺ cells from bone marrow into peripheral blood in the subject, as described herein, and the step of collecting the mobilized CD34⁺ cells from the peripheral blood of the subject. The collecting step optionally comprises apheresis. In some embodiments, the method further comprises, after the collecting step, an enriching step in which CD34⁺ cells are separated from CD34-negative (CD34⁻) cells to provide an enriched population of CD34⁺ cells. The enriching step optionally comprises the use CD34-specific antibodies or antigen-binding fragments thereof. In some embodiments, the method further comprises a formulating step in which the CD34⁺ cells are formulated into a pharmaceutical composition comprising (i) a plasma protein and (ii) an isotonic solution comprising at least one salt, in accordance with the teachings provided herein.

### DETAILED DESCRIPTION OF THE INVENTION

The invention is based in part on the discovery that compositions comprising a plasma protein and an isotonic solution containing at least one salt, e.g., but without limitation to, an isotonic solution comprising acetate, gluconate, or both acetate or gluconate, provide a stable environment for a cell population comprising CD34+ cells. Accordingly, compositions comprising a cell population comprising CD34+ cells are provided herein. The following sections are provided to describe exemplary cell populations of the pharmaceutical compositions of the invention.

Cell Populations

Generally speaking, cells of a cell population may be characterized by cell surface marker phenotype. For example, a cell population can be described as a heterogeneous cell population, wherein a certain percentage (e.g., but not limited to, at least 25%, at least 30%, at least 40%, at least 50%, at least 60%, at least 70%, at least 80%, at least 90%, or at least 95%) of the cells in the population have a common cell surface marker phenotype. In various aspects, the phenotype is the expression of a cell marker. In alternative or additional aspects, the phenotype is the lack of expression of a cell marker.

With regard to the present invention, the cell population is one which comprises cells that express the cell surface marker, CD34. In other words, the cell populations described herein comprise CD34+ cells. In exemplary embodiments, the population of cells described herein is a heterogeneous population of cells, such that not all of the cells of the population express CD34. In exemplary embodiments, the cell population comprises or is a heterogeneous cell population of which at least 1% of the cells of the cell population are CD34⁺ cells. In exemplary embodiments, the cell population comprises or is a heterogeneous cell population of which at least 5%, at least 10%, at least 15%, at least 20%, at least 25%, at least 30%, at least 35%, at least 40%, at least 45%, at least 50%, at least 55%, at least 60%, at least 65%, at least 70% at least 75%, at least 80%, at least 85%, at least 90%, at least 95%, at least 96%, at least 97%, at least 98%, or at least 99% of the cells of the cell population are CD34⁺ cells. In some embodiments, the cell population comprises a subpopulation of cells that express one or more common cell markers selected from the group consisting of CD34, CXCR4, c-kit (CD117), FLK-1 (VEGFR-1), Tie-2, KDR (VEGFR-2), CD271, CD31, CD133, CD45, CD14, CD64, CD61, CD141, CD33, CD38, CD31, CD105, CD146, CD144, CD73, CD99, CD29 and CD90. In exemplary aspects, the subpopulation of cells that express one or more of the above cell markers also express CD34. In alternative aspects, the subpopulation of cells that express one or more of the above cell markers do not express CD34.

In some embodiments, the heterogeneous population of cells comprises only CD34⁺ cells, but the population is not a clonal population, e.g., not genetically indistinct from each other. In exemplary aspects, a substantial portion of the population of cells expresses one or more common cell markers, e.g., but without limitation to, CXCR4, c-kit (CD117), FLK-1, (VEGFR-1), Tie-2, KDR (VEGFR-2), CD133, CD45, CD14, CD64, CD61, CD141, CD33, CD38, CD31, CD105, CD146, CD144, CD73, CD99, CD29 and/or CD90, but the expression levels of the one or more other cell markers is different among the cells of the population. For example, the cell population may comprise CD34+ cells that express CXCR4, as well as CD34+ cells that do not express CXCR4.

In exemplary aspects, the cell population may be considered as heterogeneous, because the cells of the cell population express CD34 to varying degrees. In exemplary aspects, the cell population may comprise cells that strongly express CD34 (i.e., “CD34 bright” cells) in addition to cells that weakly express CD34 (i.e., “CD34 dim” cells).

The cell populations described herein are, in some embodiments, purified. The term “purified,” as used herein means having been increased in purity as a result of being separated from other components of the original composition (i.e., the composition before purification). In some aspects, a purified cell population comprises at least about 10% or greater of a single type of cell. In some embodiments, the purified cell population comprises about 20%, about 30%, about 40%, about 50%, about 60%, about 70%, about 80%, about 90%, about 91%, about 92%, about 93%, about 94%, about 95%, about 96%, about 97%, about 98%, about 99% or about 100% of a single type of cell. Alternatively or additionally, a cell population may be described as “enriched,” and/or “selected.” In some embodiments, the enrichment or selection is a 1.5-fold, 2-fold, 3-fold, 4-fold, 5-fold, 6-fold, 7-fold, 8-fold, 9-fold, 10-fold, 50-fold, 100-fold, 1000-fold more of a single cell type compared to the amount or number of the same cells in an original population of cells, i.e., the population of cells before enrichment or selection. It is recognized that “purity,” “enrichment” and “selection” are relative terms, and not to be necessarily construed as absolute purity or absolute enrichment or absolute selection. In exemplary aspects, the purity is at least or about 50%, at least or about 60%, at least or about 70%, at least or about 80%, or at least or about 90% (e.g., but without limitation to, at least or about 91%, at least or about 92%, at least or about 93%, at least or about 94%, at least or about 95%, at least or about 96%, at least or about 97%, at least or about 98%, at least or about 99%) or is approximately 100%. In exemplary aspects, the enrichment or selection is at least or about 50%, at least or about 60%, at least or about 70%, at least or about 80%, or at least or about 90% (e.g., but without limitation to, at least or about 91%, at least or about 92%, at least or about 93%, at least or about 94%, at least or about 95%, at least or about 96%, at least or about 97%, at least or about 98%, at least or about 99%) or is approximately 100%, relative to the original population before enrichment or selection.

In exemplary aspects, the cell population of the invention is an enriched population of CD34+ cells. Suitable techniques to purify, enrich and/or select for CD34+ cells are known in the art and are described herein. See, e.g., International Patent Application Publication Nos. WO/2011/041478 and WO/2010/045645, which are incorporated herein by reference in their entirety. In some embodiments, the percentage of CD34+ in the enriched population is at least or about 1.5 to about 5-fold more than the percentage of CD34+ in the population of cells before selection or purification.

In exemplary aspects, the cell population is purified to the extent that at least or about 50%, at least or about 60%, at least or about 70%, at least or about 80%, or at least or about 90% (e.g., but without limitation to, at least or about 91%, at least or about 92%, at least or about 93%, at least or about 94%, at least or about 95%, at least or about 96%, at least or about 97%, at least or about 98%, at least or about 99%) or approximately 100% of the cells of the cell population are CD34+ cells.

In exemplary aspects, the cell population has undergone steps for enriching or selecting for CD34+ cells and the degree of enrichment or selection for CD34+ cells is at least or about 50%, at least or about 60%, at least or about 70%, at least or about 80%, or at least or about 90% (e.g., but without limitation to, at least or about 91%, at least or about 92%, at least or about 93%, at least or about 94%, at least or about 95%, at least or about 96%, at least or about 97%, at least or about 98%, at least or about 99%) or is approximately 100%, relative to the original population before enrichment or selection.

The degree of “enrichment” or “selection” may, in exemplary aspects, be characterized in terms of % yield, which is defined as [(the total number of cells (e.g., but without limitation to, CD34+ cells) in the post-enriched population of cells) divided by (the total number of cells (e.g., but without limitation to, CD34+ cells) in the pre-enriched population of cells)] multiplied by 100. An exemplification of % yield is provided herein as Example 2. In exemplary aspects, the degree of “purity” may be characterized in terms of % purity. Suitable methods for determining % purity are known in the art and also are provided herein as Example 2 (e.g., but without limitation to, the section entitled “Subset Analysis”).

In some aspects, the population of cells of the invention is purified of debris or dead cells.

In some embodiments, the heterogeneous population comprises other types of cells, cells other than CD34+ cells. In some aspects the heterogeneous population of cells comprises, in addition to the CD34+ cells, a white blood cell (a white blood cell of myeloid lineage or lymphoid lineage), a red blood cell, an endothelial cell, circulating endothelial precursor cells, an epithelial cell, a kidney cell, a lung cell, an osteocyte, a myelocyte, a neuron, and/or a smooth muscle cell. In exemplary aspects, the heterogeneous population comprises CD34+ cells and other cell types, but is free of red blood cells and/or platelets or has a low level of red blood cells and/or platelet. For example, less than 2% of the cells of the cell population are red blood cells and/or platelets. In exemplary aspects, the heterogeneous population comprises CD34+ cells and one or more of B-cells, T-cells, granulocytes, and monocytes and is free of red blood cells and/or platelets. In exemplary aspects, the heterogeneous population comprises mostly CD34+ cells and only a minor amount of one or more of B-cells, T-cells, granulocytes, platelets, and monocytes. For example, in exemplary aspects, greater than 75% of the heterogeneous population is CD34+ cells and less than 15% of the heterogeneous population is one or more of B-cells, T-cells, granulocytes, and monocytes. In exemplary aspects, greater than 85% of the heterogeneous population is CD34+ cells and less than 10% of the heterogeneous population is one or more of B-cells, T-cells, granulocytes, platelets and monocytes. In exemplary aspects, greater than 75%, 85%, or 95% of the heterogeneous population are CD34+ cells and no more than about 4% of the heterogeneous population are B-cells, no more than about 2% of the heterogeneous population are T cells, no more than about 1% of the heterogeneous population are monocytes, no more than about 2% of the heterogeneous population are granulocytes and/or macrophages, and/or no more than about 5% of the heterogeneous population are platelets.

A. Enriched or Selected Cell Populations

In exemplary aspects, the cell population of the pharmaceutical composition is a cell population that has been isolated from a donor and subsequently purified, enriched, or selected for CD34+ cells. The following discussion exemplifies steps to purify, enrich, or select such isolated populations for CD34+ cells.

In exemplary aspects, the cell population comprising CD34+ cells is one which has been isolated from a donor. Exemplary donors are described below. In exemplary aspects, the cell population of the pharmaceutical compositions are freshly-isolated from a donor, as described below. In exemplary aspects, the isolated cells from the donor undergo enrichment or purification or selection steps, such that the total % of CD34+ cells within the cell population is increased, relative to the original isolated but unenriched, unpurified or unselected cell population. In exemplary aspects, the enrichment or purification or selection occurs within 2 days of isolating the cell population from the donor. In exemplary aspects, the enrichment or purification or selection occurs within 1 day of isolating the cell population from the donor. In exemplary aspects, the enrichment or purification or selection occurs on the same day of isolating the cell population from the donor, but subsequent to the isolation of the cells from the donor.

In some embodiments, the population of cells is one which has undergone one or more positive selection steps, e.g., but without limitation, by immunomagnetic cell selection. In this regard, for example, the population of cells in some embodiments is enriched, selected or purified by using a primary antibody which is specific for a cell marker expressed by CD34+ cells. In some embodiments, the cell marker expressed by cells of the cell population is CD34. In exemplary embodiments, the primary antibody is an antibody which specifically binds to CD34. CD34 specific antibodies are known in the art and are commercially available. See, for example, U.S. Pat. No. 4,965,204. In some embodiments, the CD34-specific antibody is the antibody provided in an Isolex 300i kit (Baxter, Deerfield, Ill.).

In exemplary aspects, the primary antibody is contacted with the isolated (but un-enriched) cell population at a final concentration within about 0.01 μg per 10⁶ CD34+ cells and about 10 μg per 10⁶ CD34+ cells, within about 0.1 μg per 10⁶ CD34+ cells and about 5 μg per 10⁶ CD34+ cells, or within about of about 1 μg per 10⁶ CD34+ cells to about 3 μg per 10⁶ CD34+ cells. In some embodiments, the primary antibody is at a final concentration of about 2.5 μg per 10⁶ CD34+ cells.

In exemplary aspects, the primary antibody is an antibody that binds to a cell marker expressed by CD34+ cells but is a cell marker other than CD34. For example, the primary antibody may be a CD45-specific antibody or a CXCR4-specific antibody. In this regard, cells that are positive for both CD34 and CD45 and/or CXCR4 will be selected. In some aspects, a biological sample (e.g., and without limitation, blood sample) obtained from a mammalian subject is incubated with an anti-CD34⁺ antibody or antibody that selects for other epitopes/enzymes/proteins contained on or in the CD34⁺ cells.

The primary antibody which separates the population of cells into subpopulations is, in some embodiments, “captured” onto a solid support. Exemplary solid supports include, without limitation, membranes, surfaces, beads, resins, particles and other supports well known in the art. For example and without limitation, in some aspects the solid support is a bead and the bead is incubated with the population of cells obtained from the peripheral blood. In some aspects, the incubation with the bead(s) occurs before or after incubation of these cells with the primary antibody. In some embodiments, the bead(s) are incubated with the population of cells obtained from a blood sample simultaneously with the primary antibody. Once the cells of the population have been incubated with both the bead(s) and the primary antibody, complexes comprising the bead, the primary antibody, and the desired cell type or the non-desired cell form.

In exemplary aspects, the primary antibody is captured onto the solid support by way of the solid support comprising a secondary antibody which binds to the primary antibody. The secondary antibody may be on which binds to the Fc region of the primary antibody. In exemplary aspects, the solid support comprises a protein, e.g., but without limitation to, Protein A, Protein G, Protein A/G, or Protein L (e.g., but without limitation to, Protein A, Protein G, Protein A/G, or Protein L from Staphylococcus aureus) which specifically binds to the secondary antibody. In alternative aspects, the primary antibody is captured onto the solid support without a secondary antibody.

In exemplary aspects, the solid support is magnetic. In exemplary aspects, the solid support is a magnetic bead. In exemplary aspects, the magnetic beads comprise a protein which binds to the primary antibody. In specific embodiments, the protein is a secondary antibody which specifically binds to the primary antibody, e.g., but without limitation to, the Fc region of the primary antibody. In some embodiments, the protein is Protein A, Protein G, Protein A/G, or Protein L (e.g., but without limitation to, Protein A, Protein G, Protein A/G, or Protein L from Staphylococcus aureus).

In exemplary aspects, the antibody/biological sample mixture, e.g., but without limitation, antibody/blood sample mixture, is incubated with paramagnetic beads coated with antibody directed against CD34⁺ antibody. The bead-antibody complexes with the CD34⁺ cell-antibody complex, forming a cell-antibody-bead complex. This beaded complex is then separated from the remainder of the blood sample by use of a magnet in exemplary aspects. The non-magnetic bound material may then be washed away from the bound material and the resultant bound material is then incubated with a peptide, which competes for the anti-CD34⁺ antibody. Such peptide has competitive or higher affinity for the anti-CD34⁺ antibody and, consequently, the cells are released from the beads, the antibody and the magnetic. In certain embodiments, gentle mechanical agitation (e.g., but without limitation to, trituration) is used to break up clumping of the cellular complexes in the biological sample to allow the peptide to remove the antibody and bead, thereby release the cells. The peptide-antibody-bead complexes would then be removed through the use of a magnet.

In exemplary aspects, antibody selection technology (Isolex 300i, Baxter Healthcare Corp., Deerfield, Ill.) is used to isolate, purify, and harvest human CD34+ stem cells from a patient's blood or bone marrow (U.S. Pat. Nos. 5,536,475; 6,251,295; 5,968,753; 6,017,719, the disclosures of which are incorporated herein by reference in their entireties). In some aspects, the enrichment process is performed with an Isolex system, for example, but without limitation, the Isolex 300i system or modification thereof (Baxter, Deerfield, Ill.).

While some embodiments encompass the selection of CD34⁺ cells, similar processes may be employed to select for other cells, such as CXCR4⁺, Flk-1⁺ (VEGFR-1), KDR (VEGFR-2), Tie-2⁺, c-kit⁺ (CD117⁺), CD271⁺, CD133⁺, CD45⁺, CD14⁺, CD64⁺, CD61⁺, CD141⁺, CD33⁺, CD38⁺, CD105⁺, CD146⁺, CD144⁺, CD73⁺, CD99⁺, CD29⁺, CD90⁺ or CD31⁺ cells. Such similar processes involve the use of antibodies specific for these other cell markers, such as anti-CXCR4, anti-Flk-1, anti-KDR, anti-Tie-2, anti-c-kit, anti-CD271, anti-CD133, anti-CD45, anti-CD14, anti-CD64, anti-CD61, anti-CD141, anti-CD33, anti-CD38, anti-CD105, anti-CD146, anti-CD144, anti-CD73, anti-CD99, anti-CD29, anti-CD90 or anti-CD31 antibodies, for example. Presumably, in such instances, the pharmaceutical composition comprises cells which express one or more these markers. The invention contemplates the use of any of these selection processes alone, or in concert with one or more of the other processes such that the final, resultant, enhanced cell population would be enriched for one of these cells or mixtures thereof.

In some embodiments, the cell population is one which has undergone negative selection steps. In this regard, by way of example and without limitation, a biological sample is incubated with one or more antibodies directed against cells of lesser or no interest to the final product. In some embodiments, when the cells of interest are CD34⁺ cells, antibodies directed to the undesired, non-CD34⁺ (or CD34⁻) cells may be incubated with the cells obtained from the blood sample or following incubation with paramagnetic beads coated with antibodies directed against the cell-specific antibodies. Through the process described above, such undesired (non-targeted) cells are then optionally isolated from the cell population and removed. The resultant population contains lower to no concentration of the undesired cells and, consequently, a higher concentration of the desired cells, for example, CD34⁺ cells. Examples of such cell removal include the reduction or removal of cells expressing Glycophorin-a in the blood sample.

In some embodiments, the population of cells is one which has undergone both positive and negative selection steps.

In certain aspects, the method of obtaining an enriched population of CD34⁺ cells comprises separating the population of cells into a subpopulation comprising CD34⁺ cells and a subpopulation devoid of CD34⁺ cells (CD34⁻ cells) by removing the complexes comprising the beads and the primary antibody and either the CD34⁺ cell or non-CD34⁺ (CD34⁻) cell from the cell population which contained the cells, beads, and primary antibody. Methods of removing the beads are known in the art. In some embodiments, the beads are paramagnetic beads and the beads are removed with a magnet. In some embodiments, the beads are separated by centrifugation.

In some embodiments, the complexes comprising the solid support (e.g., but without limitation, beads) and primary antibody further comprises the CD34+ cells or the non-CD34 cells. In the embodiments in which the complexes comprise the non-CD34 cells, the CD34+ cells are contained in the solution from which the beads were removed. In some embodiments, no further steps are taken to enrich or purify the CD34+ cells.

In some embodiments in which the complexes as described herein comprise CD34⁺ cells, the method comprises one or more further steps to release the CD34+ cells from the complexes. In certain aspects, the method comprises incubating the complexes with a release peptide. As used herein, the term “release peptide” is any molecule comprising at least two amino acids connected via a peptide bond which displaces the primary antibody from a desired cell (such as the CD34⁺ cell).

In some embodiments, the release peptide comprises an epitope which is an epitope of CD34 or an epitope of the primary antibody, e.g., but without limitation to, a CDR of the primary antibody. In some aspects, the release peptide is a soluble CD34, (e.g., but without limitation to, a soluble fragment of CD34), or a PR34 peptide, which is described in U.S. Pat. Nos. 5,968,753 and 6,017,719. In some embodiments, the release peptide is any of those described in these patents. In some embodiments, the release peptide is one which is provided as part of the Isolex 300i Kit (Baxter, Deerfield, Ill.).

In some embodiments, the concentration of the release peptide is present with the complexes at a final concentration within about 0.01 mg/ml and 10 mg/ml, within about 0.1 mg/ml and about 5 mg/ml, or within about of 1 mg/ml to about 2 mg/ml. In some embodiments, the release peptide is at a final concentration of about 2 mg/ml.

In some embodiments, the release peptide is incubated with the complexes while rotating, shaking, or otherwise moving. In some embodiments, the release peptide is incubated without an movement.

In some embodiments, the complexes are optionally triturated to increase the efficiency of the release peptide-mediated displacement of the primary antibody from the cell (e.g., but without limitation to, CD34⁺ cell). In some embodiments, triturating is accomplished with a syringe, a pipette, or like tool which has a relatively small bore through which cells can pass and which facilitates the breaking of cell clumps formed upon complex formation. In certain aspects, the method comprises triturating for at least or about 30 seconds, at least or about 1 minute, at least or about 5 minutes, at least or about 10 minutes, at least or about 15 minutes, at least or about 25 minutes, at least or about 30 minutes, at least or about 45 minutes, at least or about 60 minutes, at least or about 90 minutes, at least or about 120 minutes at least or about 2 hours, at least or about 3 hours, at least or about 4 hours. In some aspects, the method comprises triturating for no more than about 10 hours and in other aspects, no more than about 5 hours.

In certain embodiments, trituration occurs in the presence of the release peptide. In other embodiments, trituration occurs without the release peptide present, e.g., but without limitation to, trituration occurs before addition of the release peptide. In some embodiments, trituration occurs before addition of the release peptide and trituration and the addition of the release peptide occurs within about 30 seconds, within about 60 seconds, within about 1.5 minutes, within about 2 minutes, within about 5 minutes, within about 10 minutes, within about 15 minutes, within about 30 minutes, within about 45 minutes, within about 60 minutes, of each other.

B. Cell Populations Comprising Mobilized CD34+ Cells

In exemplary aspects, the cell population comprises CD34+ cells mobilized from the bone marrow of a donor. In exemplary aspects, the cell population comprises CD34+ cells mobilized from the bone marrow of a donor treated with cytokines or other agents which induce or promote mobilization of the CD34+ cells from the bone marrow into the peripheral blood. Methods of promoting the mobilization of CD34+ cells from the bone marrow into the peripheral blood in a patient are known in the art. In exemplary aspects, the cell population comprises CD34+ cells mobilized from the bone marrow into the peripheral blood in accordance with the methods of promoting mobilization of CD34+ cells described herein.

C. Cell Populations Comprising Freshly-Isolated CD34+ Cells

In exemplary aspects, the cell population comprises cells that are freshly-isolated from a donor. By “freshly-isolated” is meant that the cells of the cell population have been existing outside the body of the donor for not more than 7 days. In exemplary aspects, the freshly isolated cells have been existing outside the body for 6 days or less, 5 days or less, 4 days or less, or 3 days or less. In exemplary aspects, the cells are freshly-isolated in accordance with the teachings below relating to methods of obtaining cell populations from a biological sample.

In exemplary aspects, the cell population is one which has been cultured or plated for less than 7 days (e.g., but without limitation to, less than 6 days, less than 5 days, less than 4 days, less than 3 days, less than 2 days, less than 1 day). In exemplary aspects, the cell population is one which has not undergone any steps for cell expansion. In exemplary aspects, the positive selection of CD34+ cells occurs within 48 hours of apheresis. The selected cells are then loaded into syringes and administered to a patient within 48 hours of being loaded into the syringes. Therefore, in exemplary aspects, the cell population has existed outside the body of the donor for less than 5 days prior to administration.

D. Methods of Obtaining Cell Populations from a Biological Sample

The cell population comprising CD34⁺ cells referenced herein may be obtained by any means known in the art. In some embodiments, the cell population is isolated from a donor. The term “isolated” as used herein means having been removed from its natural environment. The cell population is isolated from any adult, fetal or embryonic tissue comprising the desired cell population

The donor is any of the hosts described herein with regard to patients. In some aspects, the donor is a mammal. In specific aspects, the donor is a human. In some embodiments, the donor of the cell population is the same as the patient or the subject to be treated with the pharmaceutical compositions of the invention. In this regard, the cell population is considered “autologous” to the patient or subject. In other embodiments, the donor of the cell population is different from the patient or subject to be treated, but the donor and patient are of the same species. In this regard, the cell population is considered as “allogeneic.”

The cell population is isolated from any biological sample suspected of containing CD34⁺ cells. Exemplary biological samples include, but are not limited to, peripheral blood, bone marrow and adipose tissue. In exemplary aspects, the biological sample is obtained from the donor via apheresis, e.g., but without limitation, leukapheresis. In exemplary aspects, the biological sample is the mononuclear fraction obtained from the donor via apheresis (e.g., but without limitation to, leukapheresis).

In certain aspects, the cell population is isolated from the peripheral blood of the subject donor. The cell population is optionally isolated from blood following pre-treatment of the donor with cytokines or other agents which induce or promote mobilization of the cell population from the bone marrow into the peripheral blood. See Nervi et al., (J. Cell. Biochem., 99:690-705, 2006, incorporated by reference in its entirety and particularly with respect to the discussion of hematopoietic stem cell mobilization) for a review of cytokines and hematopoietic stem cell mobilization. Agents that induce or promote mobilization of the cell population from the bone marrow into the peripheral blood include, but are not limited to, granulocyte-colony stimulating factor (G-CSF), granulocyte macrophage colony stimulating factor (GM-CSF), AMD-3100, pegylated G-CSF (pegfilgrastim), thrombopoietin, stem cell factor (SCF), CXCR4 peptide (CTCE-0021; SD-1α analog), SD-1, IL-8, monocyte-chemoattractant protein-1, macrophage inflammatory protein 1a, macrophage inflammatory protein 1b, recombinant human growth hormone, recombinant human parathyroid hormone, SB-251353, Gro13 and combinations thereof. In some embodiments, one or more of AMD-3100, SCF, SB-251353, recombinant human growth hormone, and/or thrombopoietin is administered to the subject donor in combination with G-CSF (either concurrently or sequentially) to induce or promote mobilization of the cell population from the bone marrow into the peripheral blood

The invention also provides a method of obtaining CD34+ cells from a subject. The method comprises the steps of promoting mobilization of CD34⁺ cells from bone marrow into peripheral blood in the subject and collecting CD34⁺ cells from the peripheral blood of the subject. The step of promoting mobilization of CD34+ cells from bone marrow into peripheral blood of the subject comprises administering to the subject G-CSF at a total administered dose of less than 50 μg/kg. By “total administered dose” as used herein is meant the total amount administered up to the time at which collection occurs. This promoting step may be carried out in accordance with any of the teachings below. See, e.g., but without limitation to, the section entitled “Methods of promoting mobilization of CD34+ cells.” In some embodiments, the CD34⁺ cells are collected by apheresis. The method optionally further comprises, after collection of the CD34⁺ cells, enrichment of the CD34⁺ cells (as described herein). Once the CD34+ cells are obtained from the subject, the CD34⁺ cells in exemplary aspects are formulated into a pharmaceutical composition comprising a plasma protein and an isotonic solution comprising at least one salt, optionally, wherein said isotonic solution comprises acetate, gluconate, and/or both acetate and gluconate.

Methods of Promoting Mobilization of CD34+ Cells

The invention also provides a method of promoting mobilization of CD34+ cells from bone marrow into peripheral blood in a subject. The method comprises administering to the subject G-CSF at a total administered dose of less than 50 μg/kg. Without being bound to any particular theory, the inventive method minimizes the potential for pain and discomfort experienced by the subject, reduces the overall time needed for CD34+ cell-mobilization, yet allows for a sufficient number of cells to be mobilized into circulation for subsequent collection.

In some embodiments, the method of promoting mobilization of CD34⁺ cells from bone marrow into peripheral blood of the subject comprises administering G-CSF (optionally in multiple injections) to the subject at a dosage of less than or about 2 μg/kg/day, less than or about 2.5 μg/kg/day, less than or about 3 μg/kg/day, less than or about 3.5 μg/kg/day, less than or about 4 μg/kg/day, less than or about 4.5 μg/kg/day, less than or about 5 μg/kg/day, less than or about 5.5 μg/kg/day, less than or about 6 μg/kg/day, less than or about 6.5 μg/kg/day, less than or about 7 μg/kg/day, less than or about 7.5 μg/kg/day, less than or about 8 μg/kg/day, less than or about 8.5 μg/kg/day, less than or about 9 μg/kg/day, less than or about 9.5 μg/kg/day or less than or about 10 μg/kg/day immediately prior to obtaining the peripheral blood sample from the donor. In some embodiments, the method comprises administering multiple injections of G-CSF to the subject at a dosage ranging from about 2.5 μg/kg/day to about 7 μg/kg/day or from about 4 μg/kg/day to about 6 μg/kg/day). In some aspects the method comprises administering multiple injections of G-CSF to the subject at a dosage ranging from 4.8 μg/kg/day to about 5.2 μg/kg/day.

In some embodiments, less than 20 doses, or specifically, 19, 18, 17, 16, 15, 14, 13, 12, 11, 10, 9, 8, 7, 6, 5, 4, 3, 2, or 1 dose, of G-CSF is administered to the donor immediately prior to obtaining the peripheral blood sample. The method comprises, in some embodiments, administering a total of five doses of G-CSF to the donor prior to obtaining the peripheral blood sample. In other embodiments, the method comprises administering a total of four doses of G-CSF to the donor prior to obtaining the peripheral blood sample.

In exemplary aspects, the subject is administered a total administered dose of G-CSF of about 40 μg/kg or less, about 35 μg/kg or less, about 34 μg/kg or less, about 33 μg/kg or less, about 32 μg/kg or less, about 31 μg/kg or less, about 30 μg/kg or less, about 29 μg/kg or less, about 28 μg/kg or less, about 27 μg/kg or less, about 26 μg/kg or less, about 25 μg/kg or less, about 24 μg/kg or less, about 23 μg/kg or less, about 22 μg/kg or less, about 21 μg/kg or less, about 20 μg/kg or less, about 19 μg/kg or less, about 18 μg/kg or less, about 17 μg/kg or less, about 16 μg/kg or less, about 15 μg/kg or less, about 14 μg/kg or less, about 13 μg/kg or less, about 12 μg/kg or less, about 11 μg/kg or less, about 10 μg/kg or less, about 9 μg/kg or less, about 8 μg/kg or less, about 7 μg/kg or less, about 6 μg/kg or less, or about 5 μg/kg or less. In some embodiments, the total administered dose of G-CSF is administered in 7 days. In other embodiments, the total administered dose of G-CSF is administered in 5 days. In yet other embodiments, the total administered dose of G-CSF is administered in 4 days.

In exemplary aspects, the total administered dose of G-CSF is less than 50 μg/kg or is less than 30 μg/kg, and a dosage within the range of 4.8 μg/kg/day to about 5.2 μg/kg/day is administered to the subject for 4 or 5 days. In exemplary aspects, the total administered dose of G-CSF is 20 μg/kg prior to collection of peripheral blood and a dosage of about 5.0 μg/kg/day is given for four days. In exemplary aspects, the total administered dose of G-CSF is 25 μg/kg prior to collection of peripheral blood and a dosage of about 5.0 μg/kg/day is given for five days.

Pharmaceutical Compositions

The cell population, including the enriched population of CD34⁺ cells obtained from a blood sample has therapeutic value. In this regard, the invention further provides a pharmaceutical composition comprising cells for administration to a patient. The pharmaceutical composition comprises a cell population comprising CD34⁺ cells, a plasma protein and an isotonic solution comprising at least one salt.

A. Plasma Proteins

The term “plasma protein” as used herein means a protein that is present in blood plasma of a mammalian subject. Exemplary plasma proteins include, but are not limited to, albumin (in particular, human serum albumin), transferrin, haptoglobin, fibrinogen, a coagulation factor, a complement component, an immunoglobulin, an enzyme inhibitor, a precursor of substances such as angiotensin and bradykinin and other types of proteins, prealbumin, Alpha 1 antitrypsin, Alpha 1 acid glycoprotein, Alpha 1 fetoprotein, alpha2-macroglobulin, Gamma globulins, Beta 2 microglobulin, Haptoglobin, Ceruloplasmin, Complement component 3, Complement component 4, Lipoproteins, C-reactive protein (CRP), Lipoproteins (chylomicrons, VLDL, LDL, HDL), Transferrin, Prothrombin, or mannan-binding lectin (MBL, also known as mannose-binding protein or mannan-binding protein or MBP).

In exemplary aspects, the plasma protein in the pharmaceutical composition of the invention is present in the pharmaceutical composition at a concentration ranging from about 0.5% (w/v) to about 10% (w/v). In this regard, in some embodiments, the plasma protein is present in the pharmaceutical composition at a concentration of about 1% (w/v), about 2% (w/v), about 2.5% (w/v), about 3% (w/v), about 3.5% (w/v), about 4% (w/v), about 4.5% (w/v), about 5% (w/v), about 5.5% (w/v), about 6% (w/v), about 6.5% (w/v), about 7% (w/v), about 7.5% (w/v), about 8% (w/v), about 8.5% (w/v), about 9% (w/v) or about 9.5% (w/v), or about 10%. In other embodiments, the plasma protein is present in the pharmaceutical composition at a concentration ranging from about 2% (w/v) to about 10% (w/v), or from about 2% (w/v) to about 8% (w/v), or from about 3% (w/v) to about 7% (w/v) or from about 4% (w/v) to about 6% (w/v).

In certain aspects, the plasma protein in the pharmaceutical composition of the invention is serum albumin and in certain aspects, the serum albumin is human serum albumin. In some embodiments, human serum albumin is present in the pharmaceutical composition at a concentration ranging from about 0.5% (w/v) to about 10% (w/v). In this regard, in some embodiments, human serum albumin is present in the pharmaceutical composition at a concentration of about 1% (w/v), about 2% (w/v), about 2.5% (w/v), about 3% (w/v), about 3.5% (w/v), about 4% (w/v), about 4.5% (w/v), about 5% (w/v), about 5.5% (w/v), about 6% (w/v), about 6.5% (w/v), about 7% (w/v), about 7.5% (w/v), about 8% (w/v), about 8.5% (w/v), about 9% (w/v) or about 9.5% (w/v), or about 10%. In other embodiments, the human serum albumin is present in the pharmaceutical composition at a concentration ranging from about 2% (w/v) to about 10% (w/v), or from about 2% (w/v) to about 8% (w/v), or from about 3% (w/v) to about 7% (w/v) or from about 4% (w/v) to about 6% (w/v).

In exemplary embodiments, the total protein concentration attributed by the plasma protein(s) of the pharmaceutical composition is less than 60 g/L. In exemplary aspects, the total protein concentration attributed by the plasma protein(s) is less than or about 50 g/L, less than or about 40 g/L, less than or about 30 g/L, less than or about 20 g/L, or less than about 10 g/L.

In exemplary aspects, the pharmaceutical composition comprises no more than five different plasma proteins or no more than four different plasma proteins. In exemplary aspects, the pharmaceutical composition comprises no more than three different plasma proteins or no more than two different plasma proteins. In exemplary aspects, the pharmaceutical composition comprises no more than one plasma protein. In exemplary aspects, wherein the pharmaceutical composition comprises no more than five different plasma proteins, the total protein concentration attributed by the plasma protein(s) is less than 60 g/L but greater than 1 g/L, 5 g/L, 10 g/L, 15 g/L, 20 g/L, 25 g/L, 30 g/L, 35 g/L, or 40 g/L. In exemplary aspects, the total protein concentration attributed by the plasma protein(s) is between 40 g/L and 55 g/L or between 45 g/L and 53 g/L. In exemplary aspects, the total protein concentration attributed by the plasma protein(s) is about 50 g/L. In exemplary aspects, the pharmaceutical composition comprises only a single plasma protein which is human serum albumin. In exemplary aspects, the plasma proteins are recombinant proteins.

In exemplary aspects, the pharmaceutical composition comprises more than five different plasma proteins or more than ten different plasma proteins. In exemplary aspects, the pharmaceutical composition comprises all of the proteins found in plasma. In exemplary aspects, the pharmaceutical composition comprises plasma or serum. In exemplary aspects, the pharmaceutical composition comprises human plasma or human serum. In exemplary aspects, the human plasma or human serum is obtained from the human from which the CD34+ cells were isolated. In such aspects, the human plasma or human serum is considered as autologous plasma or autologous serum.

In exemplary aspects, the pharmaceutical composition comprises plasma or serum at a concentration of less than 20% (v/v). In exemplary aspects, the pharmaceutical composition comprises plasma or serum at a concentration of less than 10% (v/v). In exemplary aspects, the pharmaceutical composition comprises plasma or serum at a concentration within a range from about 2% (v/v) to about 10% (v/v), or from about 2% (v/v) to about 8% (v/v), or from about 3% (v/v) to about 7% (v/v) or from about 4% (v/v) to about 6% (v/v). In exemplary aspects, the pharmaceutical composition comprises plasma or serum at a concentration of about 1% (v/v), about 2% (v/v), about 2.5% (v/v), about 3% (v/v), about 3.5% (v/v), about 4% (v/v), about 4.5% (v/v), about 5% (v/v), about 5.5% (v/v), about 6% (v/v), about 6.5% (v/v), about 7% (v/v), about 7.5% (v/v), about 8% (v/v), about 8.5% (v/v), about 9% (v/v) or about 9.5% (v/v), or about 10% (v/v). In exemplary aspects, wherein the pharmaceutical composition comprises plasma or serum, the total protein concentration attributed by the plasma proteins is less than or about 30 g/L, less than or about 20 g/L, or less than or about 10 g/L. In exemplary aspects, wherein the pharmaceutical composition comprises plasma or serum, the total protein concentration attributed by the plasma proteins is less than or about 5 g/L, e.g., but without limitation to, between 2.5 and 4.5 g/L.

B. Isotonic Solutions

The term “isotonic solution” as used herein means a solution that has the same salt concentration as the cytoplasm of cells in the cell population. In exemplary embodiments, the isotonic solution has the same osmotic pressure as blood or has the same salt concentration as cells and blood.

In exemplary aspects of the invention, the isotonic solution comprises at least one salt. In exemplary aspects, the salt is present in the isotonic solution by way of the isotonic solution comprising separate cations and anions of the salt. In exemplary aspects, the salt is present in the isotonic solution as electrolytes.

In exemplary embodiments, the isotonic solution comprises more than one electrolyte which is present in plasma. In exemplary aspects, the isotonic solution comprises one or more of the following electrolytes: sodium (Na+), potassium (K⁺), calcium (Ca²⁺), magnesium (Mg²⁺), and chloride (Cl⁻). In exemplary aspects, the isotonic solution comprises at least sodium and one or more of potassium (K⁺), calcium (Ca²⁺), magnesium (Mg²⁺), and chloride (Cl⁻). In exemplary aspects, the isotonic soluction comprises sodium (Na+), potassium (K⁺), calcium (Ca²⁺), magnesium (Mg²⁺), and chloride (Cl⁻). In exemplary aspects, the isotonic solution comprises sodium (Na+), potassium (K⁺), calcium (Ca²⁺), and magnesium (Mg²⁺). In exemplary aspects, the isotonic solution comprises sodium (Na+), potassium (K⁺), and calcium (Ca²⁺). In exemplary aspects, the isotonic solution comprises sodium (Na+) and potassium (K⁺).

In exemplary aspects, the isotonic solution is free of at least one ion selected from sodium (Na⁺), potassium (K⁺), calcium (Ca²⁺), magnesium (Mg²⁺), and chloride (Cl⁻). In specific aspects, the isotonic solution is free of calcium (Ca²⁺).

In specific aspects, the isotonic solution comprises electrolytes or ions at a concentration which is the same as that found in plasma, e.g., but without limitation to, human plasma. Plasma contains 145 mEq/L sodium (Na⁺), 110 mEq/L chloride (Cl⁻), 4-5 mEq/L potassium (K⁺), 2 mEq/L magnesium (Mg²⁺). In exemplary aspects, the isotonic solution comprises electrolytes or ions at a concentration which is substantially the same as that found in plasma. An exemplary isotonic solution comprising electrolytes or ions at a concentration which is substantially the same as that found in plasma is one which comprises sodium (Na+) at a concentration within 10%±145 mEq/L, chloride (Cl⁻) at a concentration within 10%±110 mEq/L, potassium (K⁺) at a concentration within 10%±4-5 mEq/L, magnesium (Mg²⁺) at a concentration within 10%±2 mEq/L. Plasma also contains 5 mEq/L calcium (Ca²⁺). In exemplary aspects, the isotonic solution is free of calcium (Ca²⁺).

In certain aspects, the isotonic solution comprises sodium in an amount ranging from about 100 mEq to about 180 mEq (e.g., but without limitation to, from about 110 mEq to about 170 mEq, or from about 120 mEq to about 160 mEq, or from about 130 mEq to about 150 mEq). In some embodiments, the isotonic solution comprises sodium in an amount of about 131 mEq, about 132 mEq, about 133 mEq, about 134 mEq, about 135 mEq, about 136 mEq, about 137 mEq, about 138 mEq, about 139 mEq, about 140 mEq, about 141 mEq, about 142 mEq, about 143 mEq, about 144 mEq, about 145 mEq, about 146 mEq, about 147 mEq, about 148 mEq, about 149 mEq, or about 150 mEq. In some embodiments, the isotonic solution comprises about 140 mEq sodium.

The isotonic solution optionally comprises or further comprises potassium in an amount ranging from about 1 mEq to about 9 mEq (e.g., but without limitation to, from about 2 mEq to about 8 mEq, or from about 3 mEq to about 7 mEq, or from about 4 mEq to about 6 mEq). In some embodiments, the isotonic solution comprises potassium in an amount of about 1 mEq, about 2 mEq, about 3 mEq, about 4 mEq, about 5 mEq, about 6 mEq, about 7 mEq, about 8 mEq, or about 9 mEq. In some embodiments, the isotonic solution comprises about 5 mEq potassium.

In certain aspects, the isotonic solution optionally comprises or further comprises magnesium in an amount ranging from about 0.5 mEq to about 5.5 mEq (e.g., but without limitation to, from about 1 mEq to about 5 mEq, or from about 2 mEq to about 4 mEq). In some embodiments, the isotonic solution comprises magnesium in an amount of about 2 mEq, about 2.1 mEq, about 2.2 mEq, about 2.3 mEq, about 2.4 mEq, about 2.5 mEq, about 2.6 mEq, about 2.7 mEq, about 2.8 mEq, 2.9 mEq, about 3.0 mEq, about 3.1 mEq magnesium, about 3.2 mEq, about 3.3 mEq, about 3.4 mEq, about 3.5 mEq, 3.6 mEq, about 3.7 mEq, about 3.8 mEq, about 3.9 mEq, or about 4 mEq. In some embodiments, the isotonic solution comprises about 3 mEq magnesium.

The isotonic solution optionally comprises or further comprises, in some embodiments, chloride in an amount ranging from about 70 mEq to about 120 mEq (e.g., but without limitation to, from about 75 mEq to about 115 mEq, or from about 80 mEq to about 110 mEq, or from about 85 mEq to about 105 mEq or from about 90 mEq to about 100 mEq. In some embodiments, the isotonic solution comprises chloride in an amount of about 90 mEq, or about 91 mEq, or about 92 mEq, or about 93 mEq, or about 94 mEq, or about 95 mEq, or about 96 mEq, or about 97 mEq, or about 98 mEq, or about 99 mEq, or about 100 mEq. In some embodiments, the isotonic solution comprises about 98 mEq chloride.

In exemplary aspects, the isotonic solution comprises additional ions which are absent from plasma or which are present in plasma at very low levels.

In exemplary aspects, the isotonic solution comprises a preservative or stabilizing agent. As used herein, the term “preservative” refers to any substance which protects the pharmaceutical composition from chemical damage (e.g., but without limitation to oxidation) or microbial action. In exemplary aspects, the preservative is an anti-oxidant or an anti bacterial agent. As used herein, the term “stabilizing agent” refers to any chemical which tends to inhibit the reaction between two or more chemicals. In exemplary aspects, the stabilizing agent is an antioxidant, a sequestrant, an emulsifier or surfactant, an ultraviolet stabilizer (e.g., but without limitation to, a UV absorber, a quencher, a scavenger for free radicals. Suitable preservatives and stabilizing agents are known in the art. See, e.g., Remington's Pharmaceutical Sciences, 16^{th} edition, E. W. Martin (Mack Publishing Co., Easton Pa., 1980).

In exemplary aspects, the isotonic solution comprises a bicarbonate precursor. In exemplary aspects, the isotonic solution comprises anions which are absent from plasma, e.g., but without limitation to, human plasma, or are present in plasma at very low levels. In exemplary aspects, the bicarbonate precursor is also a preservative or a stabilizing agent and an anion which is absent from plasma or present in plasma at a very low level. In exemplary aspects, the isotonic solution comprises acetate, gluconate, or both acetate and gluconate. In exemplary aspects, the isotonic solution comprises acetate at a concentration which is greater than or about 0.06 mmol/L, greater than or about 0.07 mmol/L, greater than or about 0.08 mmol/L, greater than or about 0.09 mmol/L, or greater than or about 0.1 mmol/L. In exemplary aspects, the isotonic solution comprises acetate at an even greater concentration, e.g., but without limitation to, at a concentration of 1 mmol/L or more, 5 mmol/L or more, 10 mmol/L or more, 15 mmol/L or more, 20 mmol/L or 25 mmol/L or more.

In certain aspects, the isotonic solution optionally comprises or further comprises acetate in an amount ranging from about 10 mEq to about 40 mEq (e.g., but without limitation to, from about 15 mEq to about 35 mEq, or from about 20 mEq to about 30 mEq). In some embodiments, the isotonic solution comprises acetate in an amount of about 20 mEq, about 21 mEq, about 22 mEq, about 23 mEq, about 24 mEq, about 25 mEq, about 26 mEq, about 27 mEq, about 28 mEq, about 29 mEq, or about 30 mEq. In some embodiments, the isotonic solution comprises about 27 mEq acetate.

The isotonic solution optionally comprises or further comprises, in some embodiments, gluconate in an amount ranging from about 10 mEq to about 40 mEq (e.g., but without limitation to, from about 15 mEq to about 35 mEq or from about 20 mEq to about 30 mEq). In some embodiments, the isotonic solution comprises gluconate in an amount of about 20 mEq, about 21 about mEq, about 22 mEq, about 23 mEq, about 24 mEq, about 25 mEq, about 26 mEq, about 27 mEq, about 28 mEq, about 29 mEq or about 30 mEq. In some embodiments, the isotonic solution comprises about 23 mEq gluconate.

In certain exemplary embodiments, the isotonic solution comprises or about 140 mEq sodium, about 5 mEq potassium, about 3 mEq magnesium, about 98 mEq chloride, about 27 mEq acetate and about 23 mEq gluconate. In exemplary aspects, the isotonic solution is substantially the same as the above isotonic solution, as it comprises sodium at a concentration within about 10%±140 mEq, potassium at a concentration within about 10%±5 mEq, magnesium at a concentration within about 10%±3 mEq, chloride at a concentration within about 10%±98 mEq, acetate at a concentration within about 10%±27 mEq, and gluconate at a concentration within about 10%±23 mEq. Alternatively, the isotonic solution which is substantially the same as the above isotonice solution comprises sodium at a concentration within about 5%±140 mEq, potassium at a concentration within about 5%±5 mEq, magnesium at a concentration within about 5%±3 mEq, chloride at a concentration within about 5%±98 mEq, acetate at a concentration within about 5%±27 mEq, and gluconate at a concentration within about 5%±23 mEq.

In exemplary aspects, the osmolality of the isotonic solution ranges from, in some embodiments, about 240 mOsmol/L to about 375 mOsmol/L. The osmolality of the isotonic solution ranges from, in some embodiments, about 240 mOsmol/L to about 350 mOsmol/L (e.g., but without limitation to, from about 250 mOsmol/L to about 340 mOsmol/L, or from about 260 mOsmol/L to about 330 mOsmol/L, or from about 270 mOsmol/L to about 320 mOsmol/L or from about 280 mOsmol/L to about 310 mOsmol/L). In some embodiments, the isotonic solution has an osmolality of about 280 mOsmol/L, about 281 mOsmol/L, about 282 mOsmol/L, about 283 mOsmol/L, about 284 mOsmol/L, about 285 mOsmol/L, about 286 mOsmol/L, about 287 mOsmol/L, about 288 mOsmol/L, about 289 mOsmol/L, about 290 mOsmol/L, about 291 mOsmol/L, about 292 mOsmol/L, about 293 mOsmol/L, about 294 mOsmol/L, about 295 mOsmol/L, about 296 mOsmol/L, about 297 mOsmol/L, about 298 mOsmol/L, about 299 mOsmol/L, about 300 mOsmol/L, about 301 mOsmol/L, about 302 mOsmol/L, about 303 mOsmol/L, about 304 mOsmol/L, about 305 mOsmol/L, about 306 mOsmol/L, about 307 mOsmol/L, about 308 mOsmol/L, about 309 mOsmol/L, or about 310 mOsmol/L.

In certain aspects, the isotonic solution has a pH of about 5 to about 9 (e.g., but without limitation to, about 6 to about 8, about 6.5 to about 8 or about 7 to about 8. In some embodiments, the isotonic solution has a pH of about 7.0, about 7.1, about 7.2, about 7.3, about 7.4, about 7.5, about 7.6, about 7.7, about 7.8, about 7.9 or about 8.0.

In exemplary aspects, the isotonic solution is a crystalloid intravenous fluid comprising electrolytes. By “crystalloid intravenous fluid” is meant a sodium-based electrolyte fluid containing small molecules that flow easily across semipermeable membranes, allowing for transfer from the bloodstream into the cells and body tissues. See, e.g., but without limitation to, Crawford and Harris, “I.V. Fluids: What nurses need to know,” Nursing 41:30-38 (2011). Common crystalloid intravenous fluids are known in the art and include, but not limited to, saline, Lactated Ringer's, Ringer's solution, dextrose in water (D₅W), Darrow's solution, and 0.18% sodium chloride and 4% glucose. As used herein, the term “saline” refers to a solution of 0.90% (w/v) of NaCl.

In some embodiments, the pharmaceutical composition comprises a saline component and an autologous plasma component wherein the plasma component is at a concentration of less than about 20% (e.g., but without limitation to, about 19%, about 18%, about 17%, about 16%, about 15%, about 14%, about 13%, about 12%, about 11%, about 10%, about 9%, about 8%, about 7%, about 6%, about 5%, about 4%, about 3%, about 2% or about 1%). The pharmaceutical composition comprises, in some embodiments, saline and autologous plasma, wherein the plasma is at a concentration of about 5%.

In exemplary aspects, the isotonic solution is not saline. However, the isotonic solution in some aspects comprises sodium (Na+) with at least one other electrolyte as discussed above. In exemplary aspects, the isotonic solution is one which is free of dextrose and/or lactate and/or glucose.

In exemplary aspects, the isotonic solution is or is substantially the same as any one of: Plasma-Lyte® A, Plasma-Lyte® 148, Plasma-Lyte® 56, Normosol®-R, Isolyte® P, Lactated Ringer's solution (also known as Hartmann solution), Ringer's solution, and 5% Dextrose in water (D5W) As used herein, the term “substantially the same as” refers to a solution having the same components as that found in the reference solution, but an amount of each component which is within 10%± the amount found in the reference solution. The table below provides electrolyte content of some of these exemplary solutions.

Contents of Isotonic Solutions (mEq/L)

| Isotonic | So- | Potas- | Magne- | | Ace- | |
|---|---|---|---|---|---|---|
| Solution | dium | sium | sium | Chloride | tate | Gluconate |
| Plasma- | 140 | 5 | 3 | 98 | 27 | 23 |
| Lyte ® A | | | | | | |
| Plasma- | 140 | 5 | 3 | 98 | 27 | 23 |
| Lyte ® 148 | | | | | | |
| Plasma- | 40 | 13 | 3 | 40 | 16 | |
| Lyte ® 56 | | | | | | |
| Normosol ®-R | 140 | 5 | 3 | 98 | 27 | 23 |
| Isolyte ® P | 23 | 20 | 3 | 29 | 23 | |
| Lactated | 130 | 4 | | 109 | | |
| Ringer's | | | | | | |
| Ringer's | 147 | 4 | | 156 | | |
| solution | | | | | | |

C. Cell Populations Comprising CD34+ Cells

The pharmaceutical composition of the invention comprises a cell population comprising CD34+ cells. In exemplary aspects, the cell population is in accordance with any of the teachings of cells populations described herein. See, e.g., but without limitation to, the section entitled “Cell Populations.” Accordingly, the cell population of the pharmaceutical composition of the invention is, in exemplary aspects, a heterogeneous cell population comprising CD34+ cells and other cells. In exemplary aspects, the cell population is a heterogeneous cell population of which at least 1% (e.g., but without limitation to, at least or about 5%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 96%, at least or about 97%, at least or about 98%, or at least or about 99%) of the cells of the cell population are CD34+ cells.

Also, in exemplary aspects, the cell population of the pharmaceutical composition comprises a subset of cells that express one or more cell surface markers selected from the group consisting of: CXCR4, c-kit (CD117), FLK-1, (VEGFR-1), Tie-2, KDR (VEGFR-2), CD133, CD45, CD14, CD64, CD61, CD141, CD33, CD38, CD31, CD105, CD146, CD144, CD73, CD99, CD29 and CD90. The cells of the subset in exemplary aspects express CD34 and one or more of the above markers. In alternative aspects, the cells of the subset express one or more of the above markers but do not express CD34.

In exemplary embodiments, the pharmaceutical compositions comprises at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3.0×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4.0×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5.0×10⁶, at least about 5.5×10⁶, at least about 6.0×10⁶, at least about 6.5×10⁶, at least about 7.0×10⁶, at least about 7.5×10⁶, at least about 8.0×10⁶, at least about 8.5×10⁶, at least about 9.0×10⁶, at least about 10⁷, at least about 10⁸) cells. In exemplary aspects, the pharmaceutical composition comprises In some embodiments, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3.0×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4.0×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5.0×10⁶, at least about 5.5×10⁶, at least about 6.0×10⁶, at least about 6.5×10⁶, at least about 7.0×10⁶, at least about 7.5×10⁶, at least about 8.0×10⁶, at least about 8.5×10⁶, at least about 9.0×10⁶, at least about 10⁷, at least about 10⁸) CD34+ cells.

Without being bound to any particular theory, the pharmaceutical composition of the invention provides a stable environment for the CD34+ cells, such that the CD34+ cells may be stably stored at a non-freezing temperature (e.g., but without limitation to 1 to 30 degrees Celsius or 2 to 8 degrees Celsius) for a brief period of time (e.g., but without limitation to 2, 3, 4, or 5 days) without significantly reducing the overall viability and functionality of the population of CD34+ cells. Accordingly, the cell population in the pharmaceutical composition optionally comprises at least 70%, at least 75%, at least 80%, at least 85%, at least 90%, at least 95% or more viable cells. For example, in some embodiments, at least 70%, at least 75%, at least 80%, at least 85%, at least 90%, at least 95% or more of the cells of the cell population are viable cells after storage in the pharmaceutical composition for a period of time from about 1 hour to about 5 days and/or at a temperature within the range of 1 to 30 degrees Celsius. In exemplary aspects, at least 70%, at least 75%, at least 80%, at least 85%, at least 90%, at least 95% or more of the cells of the cell population are viable cells after storage in the pharmaceutical composition for a period of time of about 2 days and/or at a temperature within the range of 2 to 8 degrees Celsius. Cell viability of the cell population can be determined by any means known in the art, including but not limited to, the use of a viability dye such as 7-AAD. The percentage of total cells that exclude 7-AAD due to the presence of an intact cell membrane is recorded as a percentage of the total population.

Assays for measuring cell survival are known in the art, and are described, for example, by Crouch et al. (J. Immunol. Meth. 160, 81-8); Kangas et al. (Med. Biol. 62, 33843, 1984); Lundin et al., (Meth. Enzymol. 133, 2742, 1986); Petty et al. (Comparison of J. Biolum. Chemilum. 10, 29-34, 1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull J. Heterocyclic Chem. 25, 911, 1988). Assays for cell viability are also available commercially. These assays include but are not limited to CELLTITER-GLO®Luminescent Cell Viability Assay (Promega), which uses luciferase technology to detect ATP and quantify the health or number of cells in culture, and the CellTiter-Glo®. Luminescent Cell Viability Assay, which is a lactate dehyrodgenase (LDH) cytotoxicity assay (Promega).

In exemplary aspects, less than 50% of the cells of the cell population are apoptotic cells, i.e., cells undergoing apoptosis. In exemplary aspects, less than or about 40%, less than or about 30%, less than or about 20%, less than or about 10%, or less than or about 5% of the cells of the cell population are apoptotic cells. The percent of apoptotic cells in the pharmaceutical composition is determined, by for example, but without limitation, by TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.). In addition, apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface.

In exemplary aspects, the CD34+ cells of the cell population are functional CD34+ cells. In exemplary aspects, the CD34+ cells of the cell population are functional CD34+ cells after having been stored as the pharmaceutical composition for a short period of time (e.g., but without limitation to 2, 3, 4, or 5 days) at non-freezing temperatures (e.g., but without limitation to 1 to 30 degrees Celsius). Functionality of the CD34+ cells of the cell population in the pharmaceutical composition can be determined by methods known in the art, such as the migration and/or clonogenic assays described in Examples 2 and 3 herein. In some embodiments, the CD34+ cells exhibit migratory ability after having been stored for a short period of time (e.g., but without limitation to 2, 3, 4, or 5 days) at non-freezing temperatures (e.g., but without limitation to 1 to 30 degrees Celsius). In some embodiments, at least 1%, at least 5%, at least 10%, at least 15%, at least 20%, at least 25% or more of the cells in the cell population have migratory ability as determined, for example by a chemokine gradient migration assay after storing the pharmaceutical composition from about 1 hour to about 5 days, or for about 2 hours, for about 6 about, for about 12 hours, for about 1 day, for about 2 days, for about 3 days, for about 4 days or about 5 days at a temperature of about 1° C. to about 30° C. Exemplary chemokines for use in a migration assay described herein include, but are not limited to, stromal cell chemokines such as SDF-1α (CXCL-12a) and SFD-1β (CXCL-12b). See, Aiuti et al., JEM, 185:111-120, the disclosure of which is incorporated herein by reference in its entirety. Migratory ability may be characterized in terms of a “migration index” which is defined as the average CD34+ cell migration events in the sample divided by the average CD34+ cell migration events in a negative control. In exemplary aspects, the CD34+ cells of the pharmaceutical compositions have a migration index greater than that of a negative control, as determined by a migration assay (e.g., but without limitation to, the migration assay described in Example 2), after having been stored for a short period of time (e.g., but without limitation to 2, 3, 4, or 5 days) at non-freezing temperatures (e.g., but without limitation to 1 to 30 degrees Celsius). In exemplary aspects, at least 1%, at least 5%, at least 10%, at least 15%, at least 20%, at least 25% or more of the cells in the cell population demonstrate a migration index which is greater than that of a negative control, as determined by a migration assay (e.g., but without limitation to, the migration assay described in Example 2), after having been stored for a short period of time (e.g., but without limitation to 2, 3, 4, or 5 days) at non-freezing temperatures (e.g., but without limitation to 1 to 30 degrees Celsius). In exemplary aspects, the migration index is determined in a manner which slightly differs from the method described in Example 2. For instance, the migration index may be calculated for the number of all CD34+ cells, regardless of CD45 expression by these cells.

In exemplary aspects, the CD34+ cells of the cell population of the pharmaceutical composition have clonogenic potential after having been stored as a pharmaceutical composition for a short period of time (e.g., but without limitation to 2, 3, 4, or 5 days) at non-freezing temperatures (e.g., but without limitation to 1 to 30 degrees Celsius). Clonogenic potential may be measured by way of a clonogenic assay. Suitable assays for testing clonogenic potential of cells are known in the art and are described herein in Examples 2 and 3. In exemplary aspects, the CD34+ cells of the cell population of the pharmaceutical composition exhibit clonogenic potential as determined by a clonogenic assay (e.g., but without limitation to the colony forming unit (CFU) assay described herein at Example 2), after having been stored in the pharmaceutical composition for a short period of time (e.g., but without limitation to 2, 3, 4, or 5 days) at non-freezing temperatures (e.g., but without limitation to 1 to 30 degrees Celsius). In exemplary aspects, at least 1%, at least 5%, at least 10%, at least 15%, at least 20%, at least 25% or more of the CD34+ cells of the cell population of the pharmaceutical composition form colonies in a clonogenic assay (e.g., but without limitation to the colony forming unit (CFU) assay described herein at Example 2), after having been stored in the pharmaceutical composition for a short period of time (e.g., but without limitation to 2, 3, 4, or 5 days) at non-freezing temperatures (e.g., but without limitation to 1 to 30 degrees Celsius).

In certain aspects, the cell population comprises a subset of cells that co-express CXCR4⁺/CD34⁺. For example, in some embodiments, the cell population comprises from about 0.1% to about 20% CXCR4⁺/CD34⁺ cells. In some embodiments, the cell population comprises from about 0.5% to about 15%, or from about 1% to about 10%, or from about 2% to about 8%, or from about 3% to about 5% or from about 1% to about 5% or from about 1% to about 2% CXCR4+/CD34+ cells. In some embodiments, at least 0.1%, at least 0.5%, at least 1%, at least 2%, at least 3%, at least 4%, at least 5%, at least 6%, at least 7%, at least 8%, at least 9%, at least 10%, at least 15%, or at least 20% of the cells in the cell population are CXCR⁺/CD34⁺ cells.

In exemplary aspects, the CD34+ cells are CD34+ cells isolated from a human. In exemplary aspects, the CD34+ cells are CD34+ cells isolated from the peripheral blood of a human. In exemplary aspects, the human was treated with a cytokine to promote mobilization of CD34+ cells from the bone marrow to the peripheral blood of the human, prior to the time at which the CD34+ cells were isolated. In exemplary aspects, the human was administered a total administered dose of G-CSF of less than 50 μg/kg. In exemplary aspects, the G-CSF was administered at a dose between 4.8 μg/kg/day to 5.2 μg/kg/day for not more than 4 or 5 days. In exemplary aspects, the CD34+ cells are freshly isolated CD34+ cells. In exemplary aspects, the CD34+ cells have been existing outside the body of the donor for not more than 7 days, not more than 6 days, not more than 5 days, or not more than 4 days. In exemplary aspects, the CD34+ cells of the pharmaceutical composition have been formulated with the plasma protein and the isotonic solution for not more than 3 days or not more than 2 days.

D. Additional Features

The pharmaceutical composition is, in some embodiments, formulated into a ready-to-use formulation that is packaged in a non-reusable container (e.g., but without limitation to, a syringe, vial or bag). In some embodiments, the ready-to-use formulation comprises a unit dose of the pharmaceutical composition. In exemplary aspects, the pharmaceutical composition is sterile and pyrogen- and/or endotoxin-free.

Uses

The pharmaceutical compositions described herein are useful in the therapeutic treatment of diseases and medical conditions. Accordingly, the invention additionally provides a method of treating a disease or medical condition comprising administering to the patient any of the pharmaceutical compositions described herein in an amount effective to treat the disease or medical condition. The term “treat,” as well as words stemming therefrom, as used herein, does not necessarily imply 100% or complete amelioration of a targeted condition. Rather, there are varying degrees of a therapeutic effect which one of ordinary skill in the art recognizes as having a benefit. In this respect, the methods described herein provide any amount or any level of therapeutic benefit of a medical condition described herein and therefore “treat” the condition.

In some aspects, the disease or medical condition is chronic myocardial ischemia, critical limb ischemia, peripheral artery disease Berger's disease, ischemic heart disease, ischemic limb disorder, ischemic colitis, mesenteric ischemia, brain ischemia, cerebral ischemia, renal ischemia or acute limb ischemia.

By the term “ischemic limb disorder” is meant any disorder or condition that, due to primary or secondary causes, results in insufficient levels of oxygenated blood to be delivered to tissues in the extremities (arms or legs) of a mammal. Ischemic limb disorders are associated with many pathological conditions and disorders, including but not limited to atheroslcerosis, Berger's disease, critical limb ischemia, claudication, diabetic neuropathy, chemotherapy-induced neuropathy, stroke, transient ischemic attack, Parkinson's disease, and spinal cord injury.

The invention also provides a method of mediating revascularization in tissue damaged by ischemia in a subject or a method of repairing tissue damaged by ischemia in a subject. The method comprises administering any of the pharmaceutical compositions described herein in an amount effective to increase development of blood vessels in the damaged tissue or in an amount effective to repair the tissue in the subject. In some embodiments, the method results in increased development of the microvasculature in the damaged tissue. By the phrase “tissue damaged by ischemia” is meant any tissue having a deficiency in oxygen (also termed “hypoxia”) that is due to vascular disorders, such as narrowing or occlusion of an artery that supplies oxygenated blood to the tissue. Vascular disorders result in a deficiency in blood or blood vessels and can cause ischemia at any one of a number of sites including, but not limited to, cerebrovascular ischemia (e.g., but without limitation to, stroke), renal ischemia, limb ischemia (due to a circulatory disorder or limb reattachment), and organ ischemia (e.g., but without limitation to, a transplanted organ). In some embodiments, the tissue damaged by ischemia includes, but is not limited to, myocardial tissue, large intestine, small intestine, cerebral tissue, renal tissue and liver tissue. In exemplary aspects, the tissue damaged by ischemia is cardiac tissue, large intestine tissue, small intestine tissue, brain tissue, limb tissue, rental tissue or cutaneous tissue.

An individual in need of prevention, alleviation, and/or treatment of ischemia is prone to, suspected of having, or known to have tissue ischemic conditions such as those listed above. For example, individuals with circulatory problems due to organ transplant, chemotherapy treatments, diabetes, or other conditions that damage circulation may be prone to or suspected of having ischemic tissue, even if no such tissue has been observed directly. Tissues after organ transplant may also be prone to ischemia. Individuals with cardiovascular and diabetic disease can be prone to ischemia.

In some aspects in which the pharmaceutical composition comprises CD34⁺ cells, the method comprises providing therapeutic effects such as but not limited to: proangiogenic effects to combat ischemia; producing cell, tissue, and/or organ regeneration; wound healing; differentiation; reconstitution of blood supply; decrease of apoptosis; paracrine signaling, and immunomodulation. In some aspects, in which cells are selected using the positive selection procedure using antibodies directed to CD34 and CD271, the method treats inflammation. In aspects in which the pharmaceutical composition comprises CD34⁺ cells, the method provides anti-apoptotic effects.

In exemplary aspects, with regard to the methods of the invention, the CD34+ cells may be autologous cells, i.e., the donor from which the CD34+ cells were isolated is the same individual as the subject or patient being treated. In exemplary aspects, the CD34+ cells of the pharmaceutical composition were isolated from the subject no more than 2, 3, 4, 5, 6, or 7 days prior to administration to the subject. In exemplary aspects, the subject was administered a cytokine prior to the isolation of the cells. In exemplary aspects, the subject was administered a G-CSF at a total administered dose of less than 50 μg/kg prior to when the CD34+ cells were isolated from the subject. In exemplary aspects, the G-CSF was administered to the subject at a dose between 4.8 μg/kg/day and 5.2 μg/kg/day (e.g., but without limitation to, a dose of about 5±10% μg/kg per day) for not more than 4 or 5 days immediately prior to when the CD34+ cells were isolated from the subject.

Routes of Administration

In some embodiments, the pharmaceutical composition comprising the cells is formulated for parenteral administration, subcutaneous administration, intravenous administration, intramuscular administration, intra-arterial administration, intrathecal administration, or interperitoneal administration. In other embodiments, the pharmaceutical composition is administered via nasal, spray, oral, aerosol, rectal, or vaginal administration.

Methods of administering cells are known in the art. See, for example, any of U.S. Pat. Nos. 5,423,778, 5,550,050, 5,662,895, 5,800,828, 5,800,829, 5,811,407, 5,833,979, 5,834,001, 5,834,029, 5,853,717, 5,855,619, 5,906,827, 6,008,035, 6,012,450, 6,049,026, 6,083,523, 6,206,914, 6,303,136, 6,306,424, 6,322,804, 6,352,555, 6,368,612, 6,479,283, 6,514,522, 6,534,052, 6,541,024, 6,551,338, 6,551,618, 6,569,147, 6,579,313, 6,599,274, 6,607,501, 6,630,457, 6,648,849, 6,659,950, 6,692,738, 6,699,471, 6,736,799, 6,752,834, 6,758,828, 6,787,357, 6,790,455, 6,805,860, 6,852,534, 6,863,900, 6,875,441, 6,881,226, 6,884,427, 6,884,428, 6,886,568, 6,918,869, 6,933,281, 6,933,286, 6,949,590, 6,960,351, 7,011,828, 7,031,775, 7,033,345, 7,033,603, 7,049,348, 7,070,582, 7,074,239, 7,097,832, 7,097,833, 7,135,172, 7,145,055, 7,157,080, 7,166,280, 7,176,256, 7,244,242, 7,452,532, 7,470,425, and 7,494,644, the disclosures of which are incorporated by reference in their entireties.

In some embodiments, the pharmaceutical composition is delivered directly to injured tissue. The pharmaceutical composition is optionally administered by injection or by alternative delivery methods into the center, bordering zone, or neighboring areas of an ischemic tissue, e.g., but without limitation to, the myocardium, coronary blood vessels, or peripheral blood vessels. In one aspect, the pharmaceutical composition is delivered to underperfused tissue such as tissue found in chronic ischemia. Such tissue includes, but is not limited to, ischemic tissues, cardiac muscle tissues, vascular tissues, or combinations thereof. In another aspect, the cells may be introduced to an area of tissue near or within a distance sufficient to enable the cells of the pharmaceutical composition to migrate to the ischemic tissue.

Parenteral

In some embodiments, the pharmaceutical composition described herein is formulated for parenteral administration. Parenteral administration includes, but is not limited to, intravenous, intraarterial, intramuscular, intracerebral, intracerebroventricular, intracardiac, subcutaneous, intraosseous, intradermal, intrathecal, intraperitoneal, intravesical, and intracavernosal injections or infusions.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The pharmaceutical composition are in various aspects administered via a physiologically acceptable diluent in a pharmaceutical carrier, such as a sterile liquid or mixture of liquids, including water, saline, aqueous dextrose and related sugar solutions, a glycol, such as propylene glycol or polyethylene glycol, glycerol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which are optionally used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

The parenteral formulations in some embodiments contain preservatives or buffers. In order to minimize or eliminate irritation at the site of injection, such compositions optionally contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations are in various aspects presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions are in certain aspects prepared from sterile powders, granules, and tablets of the kind previously described.

Injectable formulations are in accordance with the invention. The requirements for effective pharmaceutical carriers for injectable compositions are well-known to those of ordinary skill in the art (see, e.g., but without limitation to, Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Cell Delivery Matrices

Cells obtained through the methods described herein (including CD34⁺ cells that were first mobilized from the bone marrow to the peripheral blood by a method described herein) are optionally administered via a cell delivery matrix. The cell delivery matrix in certain embodiments comprises any one or more of polymers and hydrogels comprising collagen, fibrin, chitosan, MATRIGEL, polyethylene glycol, dextrans including chemically crosslinkable or photocrosslinkable dextrans, and the like. In certain embodiments, the cell delivery matrix comprises one or more of: collagen, including contracted and non-contracted collagen gels, hydrogels comprising, for example, but not limited to, fibrin, alginate, agarose, gelatin, hyaluronate, polyethylene glycol (PEG), dextrans, including dextrans that are suitable for chemical crosslinking, photocrosslinking, or both, albumin, polyacrylamide, polyglycolyic acid, polyvinyl chloride, polyvinyl alcohol, poly(n-vinyl-2-pyrollidone), poly(2-hydroxy ethyl methacrylate), hydrophilic polyurethanes, acrylic derivatives, pluronics, such as polypropylene oxide and polyethylene oxide copolymer, 35/65 Poly(epsilon-caprolactone)(PCL)/Poly(glycolic acid) (PGA), Panacryl® bioabsorbable constructs, Vicryl® polyglactin 910, and self-assembling peptides and non-resorbable materials such as fluoropolymers (e.g., but without limitation to, Teflon® fluoropolymers), plastic, and metal.

The matrix in some instances comprises non-degradable materials, for example, but not limited to, expanded polytetrafluoroethylene (ePTFE), polytetrafluoroethylene (PTFE), polyethyleneterephthalate (PET), poly(butylenes terephthalate (PBT), polyurethane, polyethylene, polycabonate, polystyrene, silicone, and the like, or selectively degradable materials, such as poly (lactic-co-glycolic acid; PLGA), PLA, or PGA. (See also, Middleton et al., Biomaterials 21:2335 2346, 2000; Middleton et al., Medical Plastics and Biomaterials, March/April 1998, at pages 30 37; Handbook of Biodegradable Polymers, Domb, Kost, and Domb, eds., 1997, Harwood Academic Publishers, Australia; Rogalla, Minim. Invasive Surg. Nurs. 11:6769, 1997; Klein, Facial Plast. Surg. Clin. North Amer. 9:205 18, 2001; Klein et al., J. Dermatol. Surg. Oncol. 1 1:337 39, 1985; Frey et al., J. Urol. 154:812 15, 1995; Peters et al., J. Biomed. Mater. Res. 43:422 27, 1998; and Kuijpers et al., J. Biomed. Mater. Res. 51:13645, 2000).

The matrix in some embodiments includes biocompatible scaffolds, lattices, self-assembling structures and the like, whether bioabsorbable or not, liquid, gel, or solid. Such matrices are known in the arts of therapeutic cell treatment, surgical repair, tissue engineering, and wound healing. In certain aspects, the matrix is pretreated with the CD34⁺ cells. In other embodiments, the matrix is populated with CD34⁺ cells in close association to the matrix or its spaces. The CD34⁺ cells can adhere to the matrix or can be entrapped or contained within the matrix spaces. In certain aspects, the matrix-cells complexes in which the cells are growing in close association with the matrix and when used therapeutically, growth, repair, and/or regeneration of the patient's own kidney cells is stimulated and supported, and proper angiogenesis is similarly stimulated or supported. The matrix-cell compositions can be introduced into a patient's body in any way known in the art, including but not limited to implantation, injection, surgical attachment, transplantation with other tissue, and the like. In some embodiments, the matrices form in vivo, or even more preferably in situ, for example in situ polymerizable gels can be used in accordance with the invention. Examples of such gels are known in the art. or the like.

The cells in some embodiments are seeded on a three-dimensional framework or matrix, such as a scaffold, a foam or hydrogel and administered accordingly. The framework in certain aspects are configured into various shapes such as substantially flat, substantially cylindrical or tubular, or can be completely free-form as may be required or desired for the corrective structure under consideration. Two or more substantially flat frameworks in some aspects are laid atop another and secured together as necessary to generate a multilayer framework.

Examples of matrices, for example scaffolds which may be used for aspects of the invention include mats (woven, knitted, and more preferably nonwoven) porous or semiporous foams, self assembling peptides and the like. Nonwoven mats may, for example, be formed using fibers comprised of natural or synthetic polymers. In some embodiments, absorbable copolymers of glycolic and lactic acids (PGA/PLA), sold under the tradename VICRYL® (Ethicon, Inc., Somerville, N.J.) are used to form a mat. Foams, composed of, for example, poly(epsilon-caprolactone)/poly(glycolic acid) (PCL/PGA) copolymer, formed by processes such as freeze-drying, or lyophilization, as discussed in U.S. Pat. No. 6,355,699, can also serve as scaffolds. Gels also form suitable matrices, as used herein. Examples include in situ polymerizable gels, and hydrogels, for example composed of self-assembling peptides. These materials are used in some aspects as supports for growth of tissue. In situ-forming degradable networks are also suitable for use in the invention (see, e.g., but without limitation to, Anseth, K. S. et al., 2002, J. Controlled Release 78: 199-209; Wang, D. et al., 2003, Biomaterials 24: 3969-3980; U.S. Patent Publication 2002/0022676 to He et al.). These materials are formulated in some aspects as fluids suitable for injection, then may be induced by a variety of means (e.g., but without limitation to, change in temperature, pH, exposure to light) to form degradable hydrogel networks in situ or in vivo.

In some embodiments, the framework is a felt, which is comprised of a multifilament yarn made from a bioabsorbable material, e.g., but without limitation to, PGA, PLA, PCL copolymers or blends, or hyaluronic acid. The yarn in certain aspects is made into a felt using standard textile processing techniques consisting of crimping, cutting, carding and needling. The CD34⁺ cells in certain aspects are seeded onto foam scaffolds that may be composite structures. In addition, the three-dimensional framework are molded in some aspects into a useful shape, such as a specific structure in or around the ischemic tissue to be repaired, replaced, or augmented.

The framework in certain aspects is treated prior to inoculation of the CD34⁺ cells in order to enhance cell attachment. For example, prior to inoculation with the cells, nylon matrices are treated with 0.1 molar acetic acid and incubated in polylysine, PBS, and/or collagen to coat the nylon. Polystyrene is some aspects is similarly treated using sulfuric acid.

In additional embodiments, the external surfaces of the three-dimensional framework is modified to improve the attachment or growth of cells and differentiation of tissue, such as by plasma coating the framework or addition of one or more proteins (e.g., but without limitation to, collagens, elastic fibers, reticular fibers), glycoproteins, glycosaminoglycans (e.g., but without limitation to, heparin sulfate, chondroitin-4-sulfate, chondroitin-6-sulfate, dermatan sulfate, keratin sulfate), a cellular matrix, and/or other materials such as, but not limited to, gelatin, alginates, agar, agarose, and plant gums, among others.

The scaffold in some embodiments comprises materials that render it non-thrombogenic. These materials in certain embodiments promote and sustain endothelial growth, migration, and extracellular matrix deposition. Examples of such materials include but are not limited to natural materials such as basement membrane proteins such as laminin and Type IV collagen, synthetic materials such as ePTFE, and segmented polyurethaneurea silicones, such as PURSPAN® (The Polymer Technology Group, Inc., Berkeley, Calif.). These materials can be further treated to render the scaffold non-thrombogenic. Such treatments include anti-thrombotic agents such as heparin, and treatments which alter the surface charge of the material such as plasma coating.

The pharmaceutical composition comprising the cells in certain embodiments comprises any of the components of a cell delivery matrix, including any of the components described herein.

In some embodiments, the pharmaceutical composition comprises stem cells. Administration of stem cells to animals with ischemic injury is described in U.S. Pat. No. 5,980,887.

In aspects of the invention, the pharmaceutical composition comprises CD34⁺ cells. The enhanced CD34⁺ cells mixture devoid of all or substantially all of the processing reagents may then be placed in a media suitable for therapeutic injection to a patient. Such media are generally known to those skilled in the art, and may include, but are not limited to, irrigating solutions, cell culture solutions and the like. In some aspects, the CD34⁺ cells are delivered to a patient by one of several means. In some embodiments, the CD34⁺ cells are delivered intramuscularly, intra-peritoneally, intra-cranially, intra-vascularly, intravenously, between tissue components such as fractured or broken bone or cartilage.

Possible delivery options of cells include but are not limited to: direct injection (needle and syringe); injection catheter (deeper tissue); spray for surface; implanting pre-made fibrin (subcutaneous or deeper within tissue beds) optionally in conjunction with bioscaffolds (both internal and external). In aspects of the invention, the target body site for delivery can be heart, limb, eye, brain, kidney, nerve, liver, kidney, heart, lung, eye, organs of the gastrointestinal tract, skin, and brain.

Controlled Release Formulations

The pharmaceutical composition are in certain aspects modified into a depot form, such that the manner in which the pharmaceutical composition is released into the body to which it is administered is controlled with respect to time and location within the body (see, for example, U.S. Pat. No. 4,450,150). Depot forms are in various aspects, an implantable composition comprising the population of cells and a porous or non-porous material, such as a polymer, wherein the population of cells is encapsulated by or diffused throughout the material and/or degradation of the non-porous material. The depot is then implanted into the desired location within the body and the population of cells are released from the implant at a predetermined rate.

Accordingly, the pharmaceutical composition in certain aspects is modified to have any type of in vivo release profile. In some aspects of the invention, the pharmaceutical composition is an immediate release, controlled release, sustained release, extended release, delayed release, or bi-phasic release formulation.

Dose

For purposes herein, the amount or dose of the pharmaceutical composition administered is sufficient to effect, e.g., but without limitation to, a therapeutic or prophylactic response, in the subject or animal over a reasonable time frame. For example, the dose of the pharmaceutical composition is sufficient to treat or prevent a disease or medical condition in a period of from about 12 hours, about 18 hours, about 1 to 4 days or longer, e.g., but without limitation to, 5 days, 6 days, 1 week, 10 days, 2 weeks, 16 to 20 days, or more, from the time of administration. In certain embodiments, the time period is even longer. The dose is determined by the efficacy of the particular pharmaceutical composition and the condition of the recipient (or patient), as well as the body weight of the recipient (or patient) to be treated.

Many assays for determining an administered dose are known in the art. In some embodiments, an assay which comprises comparing the extent to which cells of the cell population are localized to the injured site upon administration of a given dose of such cells to a mammal among a set of mammals each of which is given a different dose of the cells is used to determine a starting dose to be administered to a mammal. The extent to which cells are localized to the injured site upon administration of a certain dose can be assayed by methods known in the art.

Also, an assay which comprises comparing the extent to which cells cause reperfusion of an injured hindlimb upon administration of a given dose of such cells to a mammal among a set of mammals each of which is given a different dose of the cells is used to determine a starting dose to be administered to a mammal. The extent to which cells cause reperfusion of an injured hindlimb upon administration of a certain dose can be assayed by methods known in the art and are described herein.

The dose of the pharmaceutical composition also will be determined by the existence, nature and extent of any adverse side effects that might accompany the administration of a particular pharmaceutical composition. Typically, the attending physician will decide the dosage of the pharmaceutical composition with which to treat each individual patient, taking into consideration a variety of factors, such as age, body weight, general health, diet, sex, therapeutic agent(s) (e.g., but without limitation to, cells) of the pharmaceutical composition to be administered, route of administration, and the severity of the condition being treated. By way of example and not intending to limit the invention, the dose of the pharmaceutical composition can be such that at least about 0.5×10⁶ cells are administered to the patient. In some embodiments, at least about 1×10⁶, at least about 1.1×10⁶, at least about 1.2×10⁶, at least about 1.3×10⁶, at least about 1.4×10⁶, at least about 1.5×10⁶, at least about 1.6×10⁶, at least about 1.7×10⁶, at least about 1.8×10⁶, at least about 1.9×10⁶, at least about 2×10⁶, at least about 2.1×10⁶, at least about 2.2×10⁶, at least about 2.3×10⁶, at least about 2.4×10⁶, at least about 2.5×10⁶, at least about 2.6×10⁶, at least about 2.7×10⁶, at least about 2.8×10⁶, at least about 2.9×10⁶, at least about 3.0×10⁶, at least about 3.1×10⁶, at least about 3.2×10⁶, at least about 3.3×10⁶, at least about 3.4×10⁶, at least about 3.5×10⁶, at least about 3.6×10⁶, at least about 3.7×10⁶, at least about 3.8×10⁶, at least about 3.9×10⁶, at least about 4.0×10⁶, at least about 4.1×10⁶, at least about 4.2×10⁶, at least about 4.3×10⁶, at least about 4.4×10⁶, at least about 4.5×10⁶, at least about 4.6×10⁶, at least about 4.7×10⁶, at least about 4.8×10⁶, at least about 4.9×10⁶, at least about 5.0×10⁶, at least about 5.5×10⁶, at least about 6.0×10⁶, at least about 6.5×10⁶, at least about 7.0×10⁶, at least about 7.5×10⁶, at least about 8.0×10⁶, at least about 8.5×10⁶, at least about 9.0×10⁶, at least about 10⁷, at least about 10⁸) cells are administered to the patient.

Timing of Administration

In exemplary aspects, the pharmaceutical composition is administered once a day, once every 2 days, once every 3 days, once every 4 days, once every 5 days, once every 6 days, once every 7 days, once every 8 days, once every 9 days, once every 10 days, once every 11 days, once every 12 days, once every 13 days, once every 14 days, once every 15 days, once every 16 days, once every 17 days, once every 18 days, once every 19 days, once every 20 days, once every 21 days, once every 22 days, once every 23 days, once every 24 days, once every 25 days, once every 26 days, once every 27 days, once every 28 days, once every 29 days, once every 30 days, or once every 31 days.

In exemplary aspects, the pharmaceutical composition is administered at a time relative to the time at which the cells were isolated from the donor. In exemplary aspects, the pharmaceutical composition is administered not more than 1 day, not more than 2 days, not more than 3 days, not more than 4 days, not more than 5 days, not more than 6 days, or not more than 7 days after the day the cells were isolated from the donor. In exemplary aspects, the pharmaceutical composition is administered on the same day the cells were isolated from the donor.

In exemplary aspects, the pharmaceutical composition is administered at a time relative to the time at which the cells were formulated into the pharmaceutical composition and loaded into the non-reusable container. In exemplary aspects, the pharmaceutical composition is administered not more than 1 day, not more than 2 days, not more than 3 days, not more than 4 days, not more than 5 days, not more than 6 days, or not more than 7 days after the day the cells were formulated into the pharmaceutical composition and loaded into the non-reusable container. In exemplary aspects, the pharmaceutical composition is administered on the same day the cells were formulated into the pharmaceutical composition and loaded into the non-reusable container.

In exemplary aspects, the pharmaceutical composition is administered at a time relative to the time at which the injury being treated occurred. In exemplary aspects, the pharmaceutical composition is administered immediately after injury. In certain embodiments of the invention, administration of the pharmaceutical composition is delayed; that is, the cells are not administered immediately after injury (e.g., but without limitation to, about 30 minutes, about 1 hour, about 2 hours, about 3 hours, about 4 hours, about 5 hours, about 6 hours, about 7 hours, about 8 hours, about 9 hours, about 10 hours, about 11 hours, or about 12 hours post-injury).

In some aspects of the invention, the pharmaceutical composition is administered to the patient at least about 12 hours, at least about 14 hours, at least about 16 hours, at least about 18 hours, at least about 20 hours, at least about 21 hours, at least about 22 hours, at least about 23 hours, at least about 24 hours, at least about 25 hours, at least about 26 hours, at least about 28 hours, at least about 30 hours, at least about 32 hours, at least about 32 hours, at least about 34 hours, at least about 36 hours, at least about 38 hours, at least about 40 hours, at least about 42 hours, at least about 44 hours, at least about 46 hours, at least about 48 hours, at least about 50 hours, at least about 52 hours, at least about 54 hours, at least about 56 hours, at least about 58 hours, at least about 60 hours, at least about 62 hours, at least about 64 hours, at least about 66 hours, at least about 68 hours, at least about 70 hours, or at least about 72 hours post-injury.

In further embodiments, the pharmaceutical composition is administered to the patient at a timepoint as described above and before about 14 days, before about 13 days, before about 12 days, before about 11 days, before about 10 days, before about 9 days, before about 8 days, before about 7 days, before about 6 days, before about 5 days, before about 4 days, or before about 3 days post-injury. In some embodiments, the pharmaceutical composition is administered to the patient at about 24 hours post-injury, or some time thereafter, but before about 14 days post-injury.

In some aspects, the pharmaceutical composition is administered after a time “X” post-injury and before a time “Y” post-injury, wherein “X” is selected from a group consisting of about 20 h, about 21 h, about 22 h, about 23 h, about 24 h, about 25 h, about 26 h, about 27 h, about 28 h, about 29 h, about 30 h, about 31 h, about 32 h, about 33 h, about 34 h, about 35 h, about 36 h, about 40 h, about 48 h, about 52 h, about 58 h, about 64 h, about 72 h, about 3.5 d, about 4 d, about 5 d, about 6 d, about 1 week, about 8 d, about 9 d, about 10 d, wherein “Y” is selected from a group consisting of about 16 d, about 15 d, about 14 d, about 13 d, about 12 d, about 11d, about 10 d, about 9 d, about 8 d, about 1 week, and wherein “X” is less than “Y.” In some aspects of the invention, the pharmaceutical composition is administered about 20, about 21, about 22, about 23, about 24 hours post-injury.

In some embodiments of the invention, the pharmaceutical composition is administered to the patient more than once. The pharmaceutical composition may be administered once daily, twice daily, three times daily, four times daily, once weekly, once every 2, 3, 4, 5, 6, 8, 9, 10, 11, 12, 13, or 14 days, or once monthly. In some embodiments, the pharmaceutical compositions is administered at or after about 24 hours post-injury and administered again at or after about 48 hours post-injury. In exemplary aspects, the timing between administrations changes as the medical condition improves in the patient or as the damaged tissue gets repaired.

Conjugates

In some embodiments of the invention, the population of cells is attached or linked to a second moiety, such as, for example, a therapeutic agent or a diagnostic agent. One or more cells in the cell population of these embodiments act as a targeting agent, since the cells are able to specifically localize to ischemic tissue. Accordingly, the invention provides in one aspect a composition comprising one or more cells of the cell population attached to a therapeutic agent or a diagnostic agent. Suitable therapeutic agents and diagnostic agents for purposes herein are known in the art and include, but are not limited to, any of those mentioned herein.

Combinations

The pharmaceutical compositions described herein, including the conjugates, are administered by itself in some embodiments. In other embodiments, the pharmaceutical compositions, including the conjugates, are administered in combination with other therapeutic or diagnostic agents. In some embodiments, the pharmaceutical composition is administered with other therapeutic agents, including, but not limited to, anti-thrombogenic agents, anti-apoptotic agents, anti-inflammatory agents, immunosuppressants (e.g., but without limitation to, cyclosporine, rapamycin), antioxidants, or other agents ordinarily used in the art to treat kidney damage or disease such as eprodisate and triptolide, an HMG-CoA reductase inhibitor (e.g., but without limitation to, simvastatin, pravastatin, lovastatin, fluvastatin, cerivastatin, and atorvastatin), cell lysates, soluble cell fractions, membrane-enriched cell fractions, cell culture media (e.g., but without limitation to, conditioned media), or extracellular matrix trophic factors.

In certain embodiments, a subset of the cell population is combined with other stem cells selected from the group consisting of totipotent stem cells, pluripotent stem cells, hematopoietic stem cells, and any other stem cells. In some embodiments, the subset of the cell population are combined with non-hematopoietic stem cells, such as, but not limited to mesenchymal cells. The cells in some embodiments are combined with scaffolds such as but not limited to fibrin, collagen, or polyethylene glycol (PEG).

The selected cells in some embodiments are used in concert with various growth factors or other bioactive agents. They could be modified using gene therapy for use as up or down regulators.

Patient Types

With regard to the inventive methods described herein, the patient is any host. In some embodiments, the host is a mammal. As used herein, the term “mammal” refers to any vertebrate animal of the mammalia class, including, but not limited to, any of the monotreme, marsupial, and placental taxas. In some embodiments, the mammal is one of the mammals of the order Rodentia, such as mice and hamsters, and mammals of the order Logomorpha, such as rabbits. In certain embodiments, the mammals are from the order Carnivora, including Felines (cats) and Canines (dogs). In certain embodiments, the mammals are from the order Artiodactyla, including Bovines (cows) and Swines (pigs) or of the order Perssodactyla, including Equines (horses). In some instances, the mammals are of the order Primates, Ceboids, or Simoids (monkeys) or of the order Anthropoids (humans and apes). In particular embodiments, the mammal is a human.

The following examples are given merely to illustrate the present invention and not in any way to limit its scope.

### EXAMPLES

### Example 1

### Mobilization of CD34⁺ Cells

The following Example evaluated factors and patterns that influence mobilization of CD34⁺ cells in human subjects suffering from chronic myocardial ischemia.

Methods:

Chronic myocardial ischemia (CMI) subjects (n=167) who were no longer candidates for surgical or interventional revascularization procedures, were mobilized with subcutaneous injections of G-CSF at a dose of 5 μg/kg/day for 5 days. Peripheral blood CD34+ cell levels were measured using flow cytometry on Days 4 and 5.

A single apheresis procedure, via a femoral or jugular placed catheter, was performed on Day 5. The number of total blood volumes (TBV) processed ranged from 2-5 and was based on each subject's Day 5 peripheral blood CD34+ cell level to minimize collection time for good mobilizers and maximize the number of CD34+ cells collected from poor mobilizers. The purpose was to harvest sufficient CD34+ cells in the apheresis mononuclear fraction (MNF) for subsequent enrichment and release testing with the goal of having adequate cells for each subject to potentially randomize to the highest dose of 50 million CD34+ cells. All but three subjects received their five doses of G-CSF. While a different apheresis device was used for 22 subjects and whole blood flow rates varied slightly, the main variable was the number of total body volumes processed during apheresis.

The apheresis MNF was enriched for CD34+ cells using the functionally closed, automated ISOLEX 300i. The CD34+ product was manually volume reduced via centrifugation and resuspended in approximately 2 mL of 0.9% normal saline, USP, containing 5% autologous plasma. A 100 μL sample was diluted to 2 mL in additional saline+5% autologous plasma. Aliquots were sent for release testing which consisted of total cell counts, hematocrit, CD34+ enumeration, total cell viability and gram stain. Other aliquots were prepared and shipped to a core facility for endotoxin and microbial testing. Subject randomization proceeded and 1 cc syringes of cells or placebo were prepared to allow 10 injections of 0.2 mL each.

Statistical Analysis:

Simple linear regression was used to test for a linear relationship between the parameters in Table 1 and the number of cells mobilized. The p-value tests the hypothesis that the slope is 0. Table 1 reports the mean and standard deviation of each parameter by four categories of the number of CD34+ cells mobilized. The same analyses were performed for the continuous parameters summarized in Table 2 (age and BMI). One way analysis of variance (AOV) was performed on the categorical parameters (gender, smoking status, and diabetic status). The analysis tests the hypothesis the number of CD34+ cells mobilized are equal for the different levels of the categorical parameters (male vs. female, smokers vs. non-smokers, diabetics vs. non-diabetics, and insulin diabetics vs. non-insulin diabetics). AOV was used to test for equal mean levels purity and yield for subjects with and without a history of congestive heart failure (CHF) in Table 5. Analysis of covariance was used to test if the linear relationship between the parameters in Table 6 and the number of CD34+ mobilized were similar in subjects with a history of CHF and those without a history of CHF. Poisson regression was used to test for equal rates of MACE in subjects with two or three planned TBVs processed and those with four or five planned TBVs processed. Fisher's exact test was used to test for equal number of subjects with at least one MACE between two to three and four to five TBVs processed.

Results:

The entire study group consisted of 87% males and 13% females ranging in age from 41-91, with an average age of 61 (±8.9) years and a mean body mass index (BMI) of 32.0 (±5.7). Ten percent were active smokers, 63% were former smokers and 27% had no history of smoking. Forty-seven percent of subjects had no history of diabetes and the remaining 53% were either insulin dependent (n−43) or not insulin dependent (n=45) diabetics.

A history of hypertension, myocardial infarction, congestive heart failure, tachycardia, pericardial effusion or pacemaker had no significant impact on an individual's ability to mobilize CD34+ cells. Comparable relationship was also observed when the data was stratified in the clinical protocol for determining the target number of TBV to the apheresed.

Results indicated that higher platelet counts correlated with good mobilization not only at baseline but throughout the mobilization period. By Day 4 and 5 of mobilization, increased white blood cell count was positively correlated with CD34 mobilization whereas the % mononuclear cells (MNCs) did not differ between poor and good mobilizers. See Table 1 below.

TABLE 1

| Day 5 Blood CD34/μL | | | | |
|---|---|---|---|---|
| <15 | 15-25 | 26-50 | >50 | P value |

Blood - Day 1 Mobilization

| | | | | | |
|---|---|---|---|---|---|
| WBC | 6.2 (1.7) | 6.4 (1.9) | 7.4 (2.0) | 6.8 (2.0) | 0.116 |
| (K/μL) | | | | | |
| Platelets | 202 (70) | 211 (57) | 229 (66) | 246 (59) | 0.005 |
| (×10⁹/L) | | | | | |
| MNC (%) | 33.7 (8.4) | 34.5 (11) | 33.9 (8.8) | 34.3 (10) | 0.680 |

Blood - Day 4 Mobilization

| | | | | | |
|---|---|---|---|---|---|
| WBC | 25.4 (6.4) | 29.8 (7.3) | 31.9 (8.6) | 31.2 (7.7) | 0.003 |
| (K/μL) | | | | | |
| Platelets | 195 (74) | 207 (53) | 218 (69) | 244 (69) | 0.005 |
| (×10⁹/L) | | | | | |
| MNC (%) | 13.7 (5.5) | 13.2 (4.4) | 14.7 (6.0) | 13.9 (5.3) | 0.512 |
| CD34 | 7.0 (3.4) | 12.8 (4.3) | 24.0 (9.9) | 41.0 (14) | <0.001 |
| (/μL) | | | | | |

Blood - Day 5 Mobilization

| | | | | | |
|---|---|---|---|---|---|
| WBC | 24.7 (7.2)00 | 31.7 (8.5) | 33.7 (7.7) | 34.1 (8.5) | <0.001 |
| (K/μL) | | | | | |
| Platelets | 189 (71) | 195 (46) | 217 (69) | 244 (75) | 0.001 |
| (×10⁹/L) | | | | | |
| MNC (%) | 15.2 (6.6) | 14.0 (5.2) | 15.6 (5.5) | 14.7 (5.7) | 0.446 |
| CD34 | 9.5 (3.6) | 19.6 (3.0) | 35.9 (7.6) | 74.0 (26) | — |
| (/μL) | | | | | |

75% of the subjects were able to mobilize ≧15 CD34+ cells/μL whole blood and required apheresis of ≦4 TBVs (Table 2) resulting in a mean total of 197×10⁶ CD34+ cells in the apheresis product. Subjects with <15 CD34+ cells/μL, whole blood apheresed 5 TBV resulting in a mean total of 76×10⁶ CD34+ cells in the apheresis product.

TABLE 2

| | Day 5 CD34/μL Blood | | | |
|---|---|---|---|---|
| | <15 | 15-25 | 26-50 | >50 |
| #TBV | 5.0 (0.2) | 4.0 (0.2) | 3.1 (0.3) | 2.2 (0.5) |
| Product Vol. | 354 (63) | 324 (64) | 276 (63) | 192 (53) |
| (mL) | | | | |
| CD34 | 220 (122) | 397 (239) | 778 (391) | 1374 (698) |
| (/μl) (MNC | | | | |
| Fraction) | | | | |
| Total CD34 | 76 (40) | 123 (76) | 206 (103) | 251 (121) |
| (×10⁶) | | | | |
| % CD34 | 98.4 (1.4) | 98.3 (2.2) | 98.5 (1.9) | 98.4 (1.5) |
| Viability | | | | |

As part of the analyses, the influence of other cardiovascular conditions was assessed and demonstrated that a history of CHF, especially in subjects requiring large volume (4-5 TBV) leukapheresis is associated with an increased rate of major adverse cardiac events (MACE) during post-treatment follow-up. Results indicated that the mobilization data of subjects with or without a history of CHF show minor numerical differences, but the differences were not statistically significant.

The analysis of subjects with greater than or less than TBVs demonstrates that the percentage of subjects with <4 TBVs developed numerically less MACE (35%) than those with ≧4 TBVs. The total number of MACE were increase >90% when subjects had >4 TBVs. When examined for the impact of a history of CHF, it was found that CHF increased rate of MACE for this group of subjects regardless of TBVs but was significantly greater for those with =4 TBVs. The group with the lowest MACE was the subjects with ≦3 TBVs and no history of CHF.

A possible alternative to excluding all subjects with a history of CHF is (1) stratify both control and treated population to receive equal amounts of such subjects; and (2) reduce the number of TBV processed during apheresis in Phase 3 given a target CD34 cell dose of 1×10⁵/kg up to 100 kg for a maximum of cell dose of 10×10⁶ CD34+ cells.

Based on the data in Table 2, subjects mobilizing <15 CD34+ cells/μL of blood on Day 5 could still collect >40 million CD34+ cells in a 3-3.5 TBV apheresis. The shorter collection time might preclude the need for a 5^{th} injection of G-CSF. Assuming 50% yield post-ISOLEX 300i (average 75% in Phase 2) and 5% of material sent for release testing, a dose of 1×10⁵ CD34+ cells/kg could be achieved in poor as well as good mobilizers.

Discussion:

The foregoing Example provides the first insight into the effect of low dose G-CSF mobilization in a CMI population of older, sedentary individuals with multiple concomitant medical conditions. Greater than 75% of all subjects mobilized ≧15 CD34+/μL of whole blood and required <4 Total body volumes during apheresis to achieve required potential of 50 million cells for treatment.

The overall Study group was 87% males and 13% females. Even though the total female numbers were small, they mobilized better than the males. This was a clear trend and though statistically significant, it was a small subject population. This observation, if it is also representative of a larger population would be in contrast to published results from normal donor population (Tigue et al., Bone Marrow Transplant., 40:185-192, 2007; Ings et al., Br. J. Haematol., 134:517-525, 2006; and Vasu et al., Blood, 112:2092-2100, 2008) or hematologic populations (Tigue et al., supra) that have been examined.

The diabetes distribution in the study was 25.7% insulin dependent, 26.9% non-insulin dependent and 47.4% with no history of diabetes. The observation that the insulin dependent subjects were statistically better mobilizers than non-insulin dependent or those with no history of diabetes is contrary to the current normal or hematologic database (Tigue, supra; Ings, supra). A recent clinical study of type 2 diabetes, however, showed that when comparing G-CSF mobilization between insulin dependent and non-insulin dependent diabetics in CAD patients that insulin dependent patients mobilized appreciably better and yielded CD34+ cells that were more functional (Humpert et al., Diabetes Care, 28:934-936, 2005). Another variable that enhanced mobilization was increased BMI which positively associated with mobilization—BMI 32±5.7.

Increased age and smoking, whether current or historical, negatively impacted mobilization. Increasing age was the most impactful factor (>65) with an average age of 61±8.9 years and age range of 41-91 years. Subjects older than 65 were observed to demonstrate a diminished capacity to mobilize sufficient cells and generally had to have more than 3 total blood volumes (TBV) to achieve or approach the total CD34+ cell count that would allow for sufficient cell recovery to yield 50 million cells. This observation is consistent with the normal donor and hematologic literature (Tigue, supra; Ings supra; and Vasu, supra) as well as the loss of functionality seen in cardiac patients for CD34+ EPCs (Dimmeler et al., Circ. Res., 102-:1319-1330, 2008). A surprising result was the impact of smoking which we observed demonstrated that non-smokers mobilized better than current or former smokers—10% active, 63% former and 27% non-smokers. This observation suggests that previous smokers even if they discontinued smoking sometime in the past, have already been negatively impacted on the ability to mobilize cells from the bone marrow via the currently understood release mechanism with G-CSF (Petit et al., Nature Immunol., 3:687-395, 2002 and Christopher et al., Blood: 114:1331-1339, 2009).

Previous studies in normal or hematologic populations (Tigue, supra; Ings supra; and Vasu, supra) did not show any substantial impact on G-CSF on cardiovascular complications or indications. The phase 2 study demonstrated a small but definite response in subjects to increases in TBVs. However, a definitive statement cannot be made that any safety risk that occurred during G-CSF treatment and apheresis was specifically due to G-CSF. The rationale behind this statement is twofold: (1) all but 3 subjects received 5 days of G-CSF and were apheresed and then a blood sample for troponin and other cardiac marker assessment together with the baseline blood draw was made. Therefore, a distinction between the effect of apheresis and G-CSF cannot be made. (2) All but 3 subjects received 5 days of G-CSF but received varying degrees of apheresis based on day 5 morning CD34 count level. Therefore, the study does not allow for the assessment of G-CSF alone. To assess impact of G-CSF on cardiovascular complications, one can examine data from the majority of cardiovascular studies (AMI) and a few chronic studies (Boyle et al., Int. J. Cardiol., 109:21-27, 2006; Losordo et al., Circ., 115″3165-3172, 2007; Humpert et al., Diabetes Care, 28:934-936, 2005; Petit et al., Nat. Immunol., 3′687-395, 2002) with limited subjects that show little negative impact of G-CSF alone. Nevertheless, there are a few studies that have presented some negative cardiac outcomes (Ripa et al., Heart Drud, 5:177-182, 2005).

The hematologic data provided in this Example demonstrates that use of 4 days of G-CSF rather than 5 days can provide more than sufficient cell numbers to reach the 10 million cell dose.

The TBVs processed during the phase 2 study were designed to maximize the ability of all subjects to reach and thus be a potential randomized candidate for the 50 millions cell dose group. The TBV was adjusted based on the day 5 CD34+ cell count and an additional injection of G-CSF was given prior to apheresis to further enhance the mobilization process. Post the phase 2 study and adverse event analysis, it was noted that MACE was elevated significantly in some subjects requiring ≧4 TBVs. A much lower MACE level was seen for those subjects who only required ≦3 TBVs of apheresis. The TBV processed during a single apheresis collection can be adjusted to minimize collection time for good mobilizers and maximize the number of CD34+ cells collected from very poor mobilizers. Identifying these characteristics and recognizing their impact should be considered when designing future cell therapy based trials utilizing mobilization and apheresis, not only in a CMI population, but other populations as well. For phase 3, we are initially recommending to lower potential risk from G-CSF by limiting to 4 doses and on day 5 have apheresis planned to not exceed 3.5 TBVs. It is contemplated that this approach will minimize potential risk to the subject, reduce time and meet the expected 10 million cell dose.

### Example 2

### Storage and Stability of Composition Comprising CD34+ Cells (Mobilized from Donors Receiving 10 μg/kg/day G-CSF)

The present Example evaluated the stability of ISOLEX selected CD34+ cells after concentration and storage in a syringe in various solutions suitable for injection.

Mobilized peripheral blood mononuclear cells (MNCs) were obtained from AllCells (Catalog#mPB026, Emeryville, Calif.) by injecting a normal healthy human donor with 10 μg/kg/day of G-CSF for 5 days followed by apheresis on Day 5 and Day 6. The mobilized MNCs were shipped at 2-8° C. under temperature-monitored conditions using 3M TL20 Temperature Loggers (St. Paul, Minn.). The average recorded temperature range was 4.5 to 8.8° C. Each donor's two mobilized MNCs were pooled and split immediately prior to paired CD34+ cell selection procedures on two ISOLEX 300i Magnetic Cell Selection Systems (version 2.5, S/N 3002=Device A and S/N 3210=Device B) with the positive selection procedure. These selections utilized Rat anti-Mouse IgG1 (RAM) paramagnetic beads and CD34⁺ antibody and PR34⁺ Releasing Agent from ISOLEX Stem Cell Reagent Kit.

Samples were collected from the pre- and post-selected product of each ISOLEX device and analyzed for cell counts, CD34+ and CD45+ cell enumeration, and total cell viability to determine yield, purity, and viability of the post ISOLEX 300i product. These test parameters were analyzed according to the following procedures:

Cell Counts:

Pre-ISOLEX samples were diluted 1:10 in Hank's Balanced Salt Solution (HBSS) and the pre- and post-ISOLEX samples were analyzed on the COULTER AcT DIFF 2 (Beckman Coulter, Pasadena, Calif., Ref. 4) in triplicate to determine the average concentration of white blood cells (WBC), red blood cells (RBC), and platelets (PLT).

CD34⁺ and CD45⁺ Cell Enumeration and Viability:

The concentration of CD34⁺ cells and CD45⁺ cells and the total cell viability were determined using Stem-Kit™. Prior to analysis, the pre-ISOLEX samples were diluted 1:10 in HBSS as not to exceed a concentration of 30×10⁶WBC/mL (the “cell suspension”). 20 μL of CD45-FITC/CD34-PE antibody was added to sample tubes (in triplicate) and 20 μL of CD45-FITC/CTRL-PE antibody was added to a control tube for each sample. Then 20 μL of 7-Amino-Actinomycin D (7-AAD) was added into each of the tubes.

Approximately 100 μL of the cell suspension was then added to each of the sample and control tubes, vortexed and incubated at room temperature in the dark for 20 minutes. Next, 2 mL of diluted lysing buffer was added to each sample tube. The sample tubes were then vortexed and incubated at room temperature in the dark for 10 minutes. 100 μL of Stem-Count Fluorospheres was added to each tube. The tubes were then vortexed and stored on melting ice in the dark. The samples were analyzed by flow cytometry within 1 hour of adding the Stem-Count Fluorospheres. The absolute CD34+ and CD45+ cell counts, percent yield, percent purity and total percentage of viable cells were then calculated as described below.

The remainder of the CD34+ selected product from each device was pooled, concentrated, and re-suspended in each of the following solutions: Formulation A (saline with 5% autologous plasma; Formulation B (PLASMA-LYTE A with 5% autologous plasma); and Formulation C (PLASMA-LYTE A with 5% HSA) to an approximate concentration of 5.0×10⁶ cells/mL (Experiments 2-6; N=5) and 7.5×10⁶ cells/mL (Experiments 1, 2, 4 and 5; N=4) as determined by Coulter cell count (±10%). The cell concentration(s) chosen for testing for each experiment was based on cell availability and was prioritized to test the cells at a concentration of 5.0×10⁶ cells/mL first. A concentration of 7.5×10⁶ cells/mL was also tested if a sufficient quantity of cells were available. The cells were loaded into 10, 1 mL syringes (Becton Dickenson, polycarbonate, luer-lock tip, Ref#309628) with a 16 gauge needle per condition tested and stored at 2-8° C. lying horizontally under static conditions over a time period of three days.

A 200 μL sample was collected from each of the syringes per condition daily (Days 0, 1, 2, and 3), pooled, and analyzed for cell counts. Prior to sample collection, the cell solution in the syringe was gently mixed. The syringe was rolled between two hands both vertically and horizontally; 15 times each. This process was repeated and followed by mixing end-over-end five times. The needle was removed and the sample was collected. The needle was replaced prior to storage of the syringes until the next time point. Cell viability, apoptosis, and purity (percentage of CD34+ cells and percentage of contaminating cell subsets) were determined. The clonogenic potential of the CD34+ cells was determined by counting the number of colonies formed using a CFU assay. Cell functionality was also analyzed on Days 1 to 3 by assessing the ability of the cells to migrate toward a chemoattractant, human stromal cell-derived factor-1α (SDF-1α). These test parameters were analyzed according to the following procedures:

Cell Counts:

Samples were analyzed on the COULTER AcT DIFF 2 (in triplicate) to determine the average concentration of white blood cells.

Viability:

The viability of the cells was determined by measuring the amount of cells that were negative for 7-Amino-Actinomycin D (7-AAD, viable) and positive for 7-AAD (non-viable) as outlined in the procedure below.
a. Added approximately 5×10⁵ cells to each sample tube (in duplicate).
b. Added 100 μL of PBS.
c. Added 10 μL of 7-AAD and incubate 10 minutes at room temperature in the dark.
d. Added 400 μL of PBS.
e. Analyzed the samples by flow cytometry.
f. Analyzed the data to determine the percentage of all events that are negative for 7-AAD (viable).

Apoptosis:

The percentage of apoptotic cells was determined using the Annexin V-FITC Apoptosis Detection Kit (BD Pharmingen, San Jose, Calif.) according to a modified version of the manufacturer's instructions as outlined below:
a. Added approximately 5×10⁵ cells to each sample tube (in duplicate).
b. Washed the cells once with 1 mL of cold PBS and re-suspend the cells in 100 μL of 1× binding buffer.
c. Added 5 μL of Annexin V-FITC and 5 μL of Propidium Iodide (PI).
d. Gently vortexed and incubated the sample for 15 minutes at room temperature in the dark.
e. Added 400 μL of 1× binding buffer and analyzed by flow cytometry.
f. Analyzed the data to determine the percentage of all events that are Annexin V+/PI− (early apoptotic) or Annexin V+/PI+ (late apoptotic or non-viable).

Subset Analysis:

Purity was determined by analyzing the sample for CD34 (stem cells), CD3/45 (T cells), CD19/45 (B cells), CD14 (monocytes), CD16 (granulocytes/macrophages), and CD61 (platelets). Analysis for the determination of cells positive for CD34/14 and CD34/61 was also conducted. The procedure for this analysis is outlined below.

Tube 1: CD3/34/45/19

Tube 2: CD16/34/61/14
a. Combined 50 μl of sample at an approximate concentration of 1×10⁶ cells/mL to 10×10⁶ cells/mL with 10 μl of each of the appropriate antibodies (in duplicate).
b. Stained and incubated the samples for 15 minutes at room temperature in the dark.
c. Washed the samples with 1 ml of 2% Fetal Bovine Serum (FBS) in PBS and resuspended in 0.5 ml of 2% FBS-PBS.
d. Analyzed the samples by flow cytometry.
e. Analyzed the data to determine the percentage of total cells positive for each of the markers described above.

Colony Forming Unit (CFU) Assay:

The CFU assay was set up (in duplicate) by first diluting cells in Iscove's Modified Dulbecco's Medium (IMDM) with 2% FBS (Stem Cells Technologies, Catalog #07700, Vancouver, Canada) and adding the diluted cells to MethoCult GF+ H4435 media (Catalog #04445, StemCell Technologies, Vancouver, Canada) for a final concentration of approximately 500 cells/dish. The CFU assays were scored using light microscopy after 14 to 16 days of incubation at approximately 37° C. and 5% CO₂ with high humidity, for the presence of Colony Forming Unit-Granulocyte Macrophage (CFU-GM), Colony Forming Unit-Erythroid (CFU-E), Burst Forming Unit-Erythroid (BFU-E), and Colony Forming Units with both GM and erythroid colonies (CFU-GEMM).

Migration Assay:

The cells were analyzed for their ability to migrate across a membrane in the presence of the chemoattractant SDF-1α according to the procedure outlined below:
a. Prepared cells to an approximate concentration of 1×10⁶ cells/mL in 0.5% HSA-RPMI.
b. Diluted SDF-1α to 10, 50, 100, 200, and 400 ng/mL in 1% HSA-RPMI and placed on ice until ready to use.
c. Pipetted 600 μL of each concentration of SDF-1α into wells (in triplicate/sample) and 600 μL 1% HSA/RPMI into three additional wells/sample to serve as negative controls.
d. Placed the Transwell inserts into wells and pipetted 100 μL of cells into each insert by dispensing cells along the side of the insert.
e. Incubated at 37° C., 5% CO₂ and high humidity for 2 to 3 hours.
f. Removed Transwell inserts from the wells and aspirated contents at bottom of plate wells and transferred into 12×75 tubes.
g. Pipetted 500 μL of 1% HSA/RPMI into wells and gently agitated.
h. Aspirated contents of wells and transferred to the corresponding tube from the first aspiration.
i. Performed flow cytometric assay as follows:
   i. To set up assay controls, pipetted 1000 of cell stock into tubes labeled Isotype, CD45-FITC, and CD34-PE.
   ii. Added 1 mL of 1% HSA-RPMI to each tube.
   iii. Centrifuged all tubes at approximately 3000 rpm for 3 minutes at room temperature.
   iv. Decanted supernatant and add 200 μL of 1% FBS-PBS to each tube.
   v. Added 10 μL of each antibody to the tubes and vortexed lightly:
      1. IgG1-FITC, IgG1-PE (cell stock)
      2. CD45-FITC (cell stock)
      3. CD34-PE (cell stock)
      4. CD45-FITC/CD34-PE (sample tubes)
   vi. Incubated the tubes in the dark for 15 minutes at room temperature.
   vii. Added 2 mL of 1% FBS/PBS to each tube and vortexed gently.
   viii. Centrifuged tubes at approximately 3000 rpm for 3 minutes at room temperature.
   ix. Decanted supernatant and added 250 μL 1% FBS/PBS to each sample tube and 500 μL to each cell stock tube.
   x. Analyzed the samples by flow cytometry.
   xi. Calculated the migration index for each sample=(Average CD34+CD45+ events in the sample/Average CD34+CD45+ events in the negative control).

All of the handling procedures were completed under aseptic conditions. This study was conducted in accordance with GLP and GDP standards.

Acceptance Criteria:

The acceptance criteria for determination of under which condition(s) the stability of the CD34+ cells was maintained was primarily based on the viability of the cells. A percent viability of greater than 80% was considered acceptable. The percentage of cells undergoing apoptosis should be less than 50%. The criterion for percent apoptosis was chosen with the premise that no data is currently available to support the clinical significance of this measurement.

The functionality of the CD34+ cells, assessed by migration and clonogenic assays, was measured with an acceptance criterion of pass or fail. Failure of the migration assay is defined as no migration of CD34+ cells in response to SDF-1α resulting in a migration index of 1.0 (same as the control) at each concentration of SDF-1α. Failure of the clonogenic assay is defined as no observable colony formation at the end of the incubation period. The samples that pass the migration and/or the clonogenic assays will undergo a qualitative assessment of the robustness of the migration of CD34+ cells in response to SDF-1α and the number of colonies observed, respectively.

Data Analysis:

The criterion of 80% for viability and 50% for apoptotic cells was tested. Analysis of variance with repeated measures was performed on these two parameters. The independent factors in the analysis were test solution, number of days stored, and the interaction between test solution and days of storage. Estimates of the least squares means were calculated along with 95% confidence intervals of these estimates for each of the test solutions and each day of storage. This analysis was completed for data collected from test formulations at both concentrations (5.0×10⁶ cells/mL and 7.5×10⁶ cells/mL). For viability, the lower bound of the 95% confidence interval was calculated. If this value was greater than 80% then the criteria was met. For apoptotic cells, the upper bound of the 95% confidence interval was calculated. If this value is less than 50% then the criteria was met.

Additional analyses were completed on each of the viability and apoptosis data sets to calculate the differences between the least square means for each pair of solutions at each day of analysis. A resultant p-value of less than 0.05 provided evidence that they are not equal.

The criteria for the migration and clonogenic assays were met if there were no failures.

Results:

Six paired ISOLEX 300i selections were completed in order to select CD34+ cells for use in this study. In each procedure, pooled and split mobilized MNCs from the same donor were run on two ISOLEX 300i Magnetic Cell Selection Systems (Device A and Device B) side-by-side with RAM beads.

Cell Count:

Triplicate samples were collected from the pre- and post-product of each ISOLEX 300i device and analyzed to determine the concentration of white blood cells (WBCs), red blood cells (RBCs), and platelets (PLTS). The total cells per unit (product loaded onto each ISOLEX device) were calculated. The average results of the total WBCs/unit are summarized in Table 1 below.

A range of 3.69×10¹⁰ to 5.01×10¹⁰ WBC/unit was loaded onto the ISOLEX 300i device. These units contained a total of 82.3×10⁹ to 154.7×10⁹ RBCs and 3.2×10¹¹ to 5.1×10¹¹ PLTS. Overall the WBC counts of the post ISOLEX 300i products averaged to 3.51×10⁸/unit with a range that encompassed 1.55×10⁸ to 5.76×10⁸ WBC/unit. None of the post ISOLEX 300i products was found to contain any measurable levels of RBCs or PLTS based on Coulter cell counts.

The total number of CD34+ and CD45+ cells in each of the pre- and post-samples were determined by flow cytometric analysis. This measurement provided a precise determination of the total number of these cell types in each pre- and post-ISOLEX 300i products. The CD34+ and CD45+ cell enumeration data is shown below in Tables 3 and 4, respectively.

TABLE 3
Enumeration of CD34+ Cells/Unit

| | Pre | | Post | |
|---|---|---|---|---|
| Experiment | Device A | Device B | Device A | Device B |
| 1 | 4.81E+08 | 4.44E+08 | 2.91E+08 | 2.77E+08 |
| 2 | 9.28E+08 | 1.01E+09 | 4.31E+08 | 4.51E+08 |
| 3 | 3.21E+08 | 3.99E+08 | 2.20E+08 | 2.29E+08 |
| 4 | 5.70E+08 | 5.67E+08 | 3.86E+08 | 3.99E+08 |
| 5 | 3.88E+08 | 3.48E+08 | 2.44E+08 | 2.76E+08 |
| 6 | 2.19E+08 | 1.75E+08 | 1.22E+08 | 1.33E+08 |
| Average | 4.84E+08 | 4.91E+08 | 2.82E+08 | 2.94E+08 |

TABLE 4
Enumeration of CD45+ Cells/Unit

| | Pre | | Post | |
|---|---|---|---|---|
| Experiment | Device A | Device B | Device A | Device B |
| 1 | 3.80E+10 | 3.82E+10 | 3.11E+08 | 2.81E+08 |
| 2 | 4.52E+10 | 5.10E+10 | 4.61E+08 | 4.70E+08 |
| 3 | 3.22E+10 | 3.87E+10 | 2.38E+08 | 2.32E+08 |
| 4 | 4.28E+10 | 4.22E+10 | 4.02E+08 | 4.53E+08 |
| 5 | 4.90E+10 | 4.90E+10 | 2.57E+08 | 2.85E+08 |
| 6 | 5.02E+10 | 4.73E+10 | 1.37E+08 | 1.50E+08 |
| Average | 4.29E+10 | 4.44E+10 | 3.01E+08 | 3.12E+08 |

In order to determine the yield of CD34+ cells obtained from each ISOLEX 300i selection, the total number of CD34+ cells in each of the post ISOLEX 300i products was compared to the values obtained for the corresponding pre samples with the following equation:

$$\% Yield = \frac{Total CD 34 + cells in the post ISOLEX 300 i product}{Total CD 34 + cells in pre ISOLEX 300 i product} \times 100$$

The enumeration data was also analyzed to determine the percent purity of each of the post ISOLEX 300i products.

The yield of CD34+ cells from each of the ISOLEX 300i selections ranged from 44.66% to 79.21%. The average yield obtained with Device A and Device B was 60.37% and 64.97%, respectively. The average purity of the post ISOLEX product was approximately 93% with each of the devices and ranged from 88.06% to 98.59%. Both the yield and the purity were fairly consistent between the paired ISOLEX 300i runs for the majority of the selection procedures. This data is displayed in Table 5.

TABLE 5
% Yield and % Purity of CD34+ Cells

| | % Yield | | % Purity | |
|---|---|---|---|---|
| Experiment | Device A | Device B | Device A | Device B |
| 1 | 60.49 | 62.35 | 93.51 | 98.59 |
| 2 | 46.39 | 44.66 | 93.50 | 95.95 |
| 3 | 68.60 | 57.26 | 92.61 | 98.53 |
| 4 | 67.74 | 70.34 | 96.01 | 88.06 |
| 5 | 63.00 | 79.21 | 94.93 | 96.55 |
| 6 | 55.98 | 75.99 | 89.45 | 88.36 |
| Average | 60.37 | 64.97 | 93.34 | 94.34 |
| SD | 0.08 | 0.13 | 0.02 | 0.05 |

The post-ISOLEX 300i product from each device was pooled, concentrated, and re-suspended to a target concentration of 5.0×10⁶ cells/mL and/or 7.5×10⁶ cells/mL in each of the following solutions: Formulation A (saline with 5% autologous plasma); Formulation B (PLASMA-LYTE A with 5% autologous plasma) and Formulation C (PLASMA-LYTE A with 5% HSA). The concentration of each of the test formulations was adjusted to within ±10% of the 5.0×10⁶ cells/mL or 7.5×10⁶ cells/mL target concentration and confirmation of the WBC concentration determined by analysis with the Coulter Act Diff 2. The CD34+ cells were adjusted to the appropriate concentration in the various test formulations and stored in syringes for up to 3 days under refrigerated (2-8° C.) conditions. Each of the test formulations were sampled daily (Days 0, 1, 2, and 3). The samples from each of the syringes for each condition were pooled and analyzed to determine the WBC concentration.

Immediately after loading the syringes for all of the time points, the Day 0 syringes were mixed by following a consistent process of rolling each syringe between two hands both vertically and horizontally; 15 times each. This process was repeated and followed by mixing end-over-end five times. Interestingly, the average WBC concentration observed on the Day 0 sample with each of the test formulation was lower than the target concentration (5.0×10⁶ cells/mL or 7.5×10⁶ cells/mL). The test formulations prepared to 5.0×10⁶ cells/mL ranged from 3.8×10⁶ cells/mL to 4.2×10⁶ cells/mL at the time of sampling (Day 0), while the test formulations prepared to 7.5×10⁶ cells/mL ranged from 5.5×10⁶ cells/mL to 6.5×10⁶ cells/mL. The average WBC concentration observed on Days 1, 2, and 3, was generally higher from that observed on Day 0 and slightly increased over time. This suggests the complexity of achieving a uniformly mixed sample of cells while they are stored in a syringe.

The average number of cells counted in each of the pooled samples/day was totaled in order to determine the percent recovery of cells out of the syringes over the three day sampling time. The results of this analysis determined that the majority of the cells (range of 89% to 98%) were recovered over the three day storage period.

On Days 0 to 3, the pooled cell samples were subsequently analyzed for viability, apoptosis, purity, and functionality by analysis of their clonogenic potential. The functionality of the CD34+ cells was also analyzed on Days 1, 2, and 3 by assessing their migration to a chemoattractant, SDF-1α. The data presented herein represents the average values of the results from each of the experiments that were completed with data obtained after storage of cells up to 3 days.

Cell Viability:

The viability of the cells was determined with the viability dye, 7-AAD. 7-AAD is a membrane impermeable dye that can only be detected in non-viable cells with compromised membranes. The percentage of total cells that excluded 7-AAD due to the presence of an intact cell membrane was recorded as a percentage of the total population. The viability of the cells prepared at both cell concentrations (5.0×10⁶ cells/mL and 7.5×10⁶ cells/mL) resulted in very similar outcomes at each time point of analysis. The viability of the cells on Day 0 ranged from 96.38% to 97.20%. Cells stored in Formulations B and C maintained high levels of viability for up to three days with an average viability of approximately 95% at that time. Viability of the cells stored in Formulation A started to decline after Day 1 with an average viability of approximately 91% on Day 2 and approximately 87% on Day 3. See Table 6 below.

TABLE 6
Average Percent Total Cell Viability in Formulations A-C

| Day | Formulation A | Formulation B | Formulation C |
|---|---|---|---|
| | Starting Concentration of 5.0 × 10⁶ cells/mL | | |
| 0 | 96.38 ± 0.59 | 96.45 ± 0.42 | 96.95 ± 0.63 |
| 1 | 94.29 ± 1.67 | 95.88 ± 0.84 | 96.68 ± 0.45 |
| 2 | 90.83 ± 2.60 | 95.43 ± 0.96 | 95.95 ± 0.98 |
| 3 | 87.62 ± 5.05 | 94.62 ± 0.44 | 95.15 ± 0.71 |
| | Starting Concentration of 7.5 × 10⁶ cells/mL | | |
| 0 | 96.63 ± 0.50 | 96.54 ± 1.13 | 97.20 ± 0.88 |
| 1 | 95.39 ± 0.92 | 96.56 ± 0.94 | 96.99 ± 0.75 |
| 2 | 90.87 ± 0.87 | 96.19 ± 0.80 | 96.80 ± 0.57 |
| 3 | 87.10 ± 2.55 | 95.24 ± 0.87 | 94.97 ± 0.57 |

The acceptance criterion for determination of the solution most suitable for storage of the CD34+ cells was primarily based on the viability of the cells. As defined in the acceptance criteria, if the lower bound of these intervals is greater than 80% then the cells may be stored in that solution at that concentration for the given number of days. This acceptance criterion was met for storage of cells in each of the solutions at both concentrations for up to three days.

Additional statistical analyses were completed on this data set to calculate the differences between the least square means for each pair of solutions at each day of analysis. The results of this analysis determined that, at both cell concentrations, there were significant differences between Formulation A and Formulation C on Day 1 as well as significant differences between Formulation A and both Formulations B and C on Days 2 and 3.

The viability of the cells was further explored by determining the percentage of cells undergoing the various stages of apoptosis versus those that were non-viable. This analysis was completed for the entire cell population present in the post-ISOLEX 300i product (regardless of cell surface markers) by staining with Annexin V and PI. Annexin V is a protein with a strong, specific affinity to phosphatidylserine (PS), a component of the phospholipid bilayer of cell membranes. In the early stages of apoptotic cell death, the phospholipid asymmetry of the plasma membrane is disrupted and PS is translocated to the outer layer of the cell plasma membrane. PS exposed on the cell membrane binds Annexin V allowing for the detection of apoptotic cells. At this stage, the cell membrane remains intact. Therefore, staining with Annexin V was completed in conjunction with PI, a membrane impermeable dye, which is similar to 7-AAD, and can only be detected in cells with compromised membranes. This allowed for the identification of early apoptotic cells (Annexin V+/PI−) vs. cells undergoing the latest stages of cell death resulting from either apoptotic or necrotic processes. At this stage, the cell membrane integrity becomes compromised and the cells will stain both with Annexin V and PI. The average percentage of late apoptotic or non-viable cells is shown in Table 7 below.

TABLE 7
Average Percentage of Late Apoptotic/Non-
Viable Cells in Formulations A-C

| Day | Formulation A | Formulation B | Formulation C |
|---|---|---|---|
| | Starting Concentration of 5.0 × 10⁶ cells/mL | | |
| 0 | 3.07 ± 0.34 | 3.22 ± 0.98 | 2.20 ± 0.37 |
| 1 | 3.21 ± 0.70 | 3.21 ± 0.72 | 2.72 ± 0.61 |
| 2 | 3.92 ± 1.69 | 2.67 ± 0.82 | 2.43 ± 0.39 |
| 3 | 4.80 ± 1.57 | 2.78 ± 0.77 | 3.43 ± 0.72 |
| | Starting Concentration of 7.5 × 10⁶ cells/mL | | |
| 0 | 2.23 ± 0.89 | 2.90 ± 1.43 | 2.10 ± 0.57 |
| 1 | 3.32 ± 1.25 | 2.98 ± 1.06 | 2.42 ± 0.51 |
| 2 | 4.29 ± 1.06 | 2.80 ± 0.60 | 2.48 ± 0.59 |
| 3 | 3.69 ± 0.60 | 2.45 ± 0.28 | 3.44 ± 0.66 |

As indicated in Table 6, on Day 0, an average of approximately 2% to 3% of the cells in each the solutions were determined to be in the late stages of apoptosis (or non-viable). The percentage of late apoptotic (or non-viable) cells remained under approximately 5% for each of the test formulations, regardless of starting cell concentration, for the duration of the storage time (until Day 3). These results correlate well with the results observed with the viability analysis with 7-AAD (Tables 6 and 7).

The percentage of early apoptotic cells in each of the test formulations were identified by Annexin V positive and PI negative staining. The results of this analysis are displayed below in Table 8 below.

TABLE 8
Average Percentage of Early Apoptotic Cells in Formulations A-C

| Day | Formulation A | Formulation B | Formulation C |
|---|---|---|---|
| | Starting Concentration of 5.0 × 10⁶ cells/mL | | |
| 0 | 3.70 ± 1.53 | 3.45 ± 1.65 | 5.43 ± 2.70 |
| 1 | 5.76 ± 1.77 | 3.53 ± 1.46 | 5.45 ± 2.18 |
| 2 | 8.44 ± 4.35 | 3.66 ± 2.01 | 7.05 ± 2.73 |
| 3 | 13.62 ± 6.63 | 7.12 ± 3.00 | 12.00 ± 3.85 |
| | Starting Concentration of 7.5 × 10⁶ cells/mL | | |
| 0 | 2.94 ± 0.58 | 2.32 ± 0.44 | 3.63 ± 1.24 |
| 1 | 4.65 ± 0.27 | 2.86 ± 0.52 | 3.92 ± 0.86 |
| 2 | 7.24 ± 1.95 | 3.05 ± 1.03 | 5.40 ± 1.50 |
| 3 | 10.95 ± 3.12 | 5.40 ± 1.70 | 9.73 ± 3.32 |

As indicated in Table 8, on Day 0, the average percentage of the cells in the early stages of apoptosis ranged from 2.32% to 5.43% in each of the test formulations (at both concentrations tested). The levels of early apoptosis observed with cells stored for one day were very similar to that observed on Day 0 regardless of the test solution utilized. The cell integrity was most stable overall during storage of the cells in Formulation B with less than 7% of cells in the early stages of apoptosis until Day 3. Analysis of cells stored in Formulation C determined that ≦12% of cells were in the early stages of apoptosis by Day 3. Cells stored in Formulation A resulted in similar results to that observed with cells stored in Formulation C (up to approximately 14% by Day 3).

Additional statistical analyses were completed on this data set to calculate the differences between the least square means for each pair of solutions at each day of analysis. The results of this analysis determined that, at both cell concentrations, there were significant differences between Formulation B and both Formulation A and Formulation C on Days 2 and 3.

Purity of Samples:

An extensive subset analysis was completed in order to examine the purity of the samples at each time point. The percentage of the cells positive for CD34 (stem cells), CD3 (T cells), CD19 (B cells), CD14 (monocytes), CD16 (granulocytes/macrophages), and CD61 (platelets) was determined. In addition, cells that were dual positive for CD61/34 (co-aggregates) and CD14/34 (potential endothelial progenitor cells; EPCs) was determined. The results are summarized in Tables 9-10 (below), for each of the test formulations at both starting concentrations (5.0×10⁶ cells/mL and 7.5×10⁶ cells/mL, respectively).

TABLE 9
Average Percentage of Cell Subsets in Formulations A, B and C (Starting
Concentration of 5.0 × 10⁶ cells/mL)

| | | | | | | | | | |
|---|---|---|---|---|---|---|---|---|---|
| Day | CD34+ | CD3+ | CD19+ | CD14+ | CD16+ | CD14/34+ | CD61+ | CD61/34+ | Total |

Formulation A

| | | | | | | | | | |
|---|---|---|---|---|---|---|---|---|---|
| 0 | 96.69 | 0.38 | 2.84 | 0.16 | 0.71 | 0.13 | 1.53 | 1.57 | 100.63 |
| 1 | 97.22 | 0.31 | 1.75 | 0.01 | 0.59 | 0.05 | 1.82 | 1.88 | 99.33 |
| 2 | 97.56 | 0.31 | 1.45 | 0.03 | 0.73 | 0.04 | 1.86 | 1.99 | 99.86 |
| 3 | 97.23 | 0.28 | 0.85 | 0.03 | 1.01 | 0.20 | 2.31 | 2.42 | 98.76 |

Formulation B

| | | | | | | | | | |
|---|---|---|---|---|---|---|---|---|---|
| 0 | 97.17 | 0.26 | 2.44 | 0.05 | 0.51 | 0.07 | 1.50 | 1.57 | 100.25 |
| 1 | 97.28 | 0.34 | 2.17 | 0.03 | 0.54 | 0.05 | 1.69 | 1.76 | 100.04 |
| 2 | 97.84 | 0.23 | 2.15 | 0.02 | 0.51 | 0.01 | 1.34 | 1.45 | 100.57 |
| 3 | 96.87 | 0.30 | 2.20 | 0.02 | 0.66 | 0.17 | 1.70 | 1.93 | 99.52 |

Formulation C

| | | | | | | | | | |
|---|---|---|---|---|---|---|---|---|---|
| 0 | 96.62 | 0.79 | 2.38 | 0.09 | 0.79 | 0.09 | 1.88 | 1.98 | 100.30 |
| 1 | 97.25 | 0.88 | 1.63 | 0.11 | 0.79 | 0.17 | 3.24 | 3.26 | 100.41 |
| 2 | 96.95 | 0.55 | 1.77 | 0.13 | 0.87 | 0.09 | 2.02 | 1.99 | 100.24 |
| 3 | 96.97 | 1.00 | 1.83 | 0.04 | 0.88 | 0.27 | 1.33 | 1.56 | 100.02 |

TABLE 10
Average Percentage of Cell Subsets in Formulations A, B and C
(Starting Concentration of 7.0 × 10⁶ cells/mL)

| | | | | | | | | | |
|---|---|---|---|---|---|---|---|---|---|
| Day | CD34+ | CD3+ | CD19+ | CD14+ | CD16+ | CD14/34+ | CD61+ | CD61/34+ | Total |

Formulation A

| | | | | | | | | | |
|---|---|---|---|---|---|---|---|---|---|
| 0 | 97.88 | 0.20 | 2.02 | 0.01 | 0.11 | 0.11 | 1.01 | 1.16 | 100.08 |
| 1 | 98.37 | 0.30 | 1.86 | 0.01 | 0.35 | 0.06 | 1.34 | 1.42 | 100.80 |
| 2 | 98.31 | 0.46 | 1.39 | 0.07 | 0.46 | 0.05 | 1.42 | 1.47 | 100.65 |
| 3 | 97.02 | 0.82 | 1.82 | 0.00 | 0.54 | 0.07 | 1.89 | 1.90 | 100.00 |

Formulation B

| | | | | | | | | | |
|---|---|---|---|---|---|---|---|---|---|
| 0 | 97.89 | 0.28 | 2.25 | 0.00 | 0.12 | 0.02 | 1.04 | 1.27 | 100.20 |
| 1 | 97.97 | 0.29 | 2.03 | 0.00 | 0.26 | 0.02 | 1.30 | 1.43 | 100.39 |
| 2 | 98.13 | 0.44 | 2.15 | 0.00 | 0.33 | 0.03 | 1.22 | 1.29 | 100.97 |
| 3 | 97.73 | 0.32 | 2.55 | 0.00 | 0.25 | 0.09 | 1.55 | 1.57 | 100.50 |

Formulation C

| | | | | | | | | | |
|---|---|---|---|---|---|---|---|---|---|
| 0 | 97.75 | 0.57 | 2.35 | 0.01 | 0.22 | 0.07 | 1.35 | 1.49 | 100.76 |
| 1 | 98.32 | 0.59 | 1.79 | 0.00 | 0.23 | 0.06 | 1.36 | 1.52 | 100.76 |
| 2 | 98.25 | 0.64 | 1.76 | 0.16 | 0.37 | 0.07 | 1.42 | 1.48 | 101.12 |
| 3 | 98.14 | 0.69 | 1.51 | 0.00 | 0.29 | 0.14 | 1.64 | 1.64 | 100.61 |

As indicated in Tables 9 and 10 above, on Day 0, each of the test formulations was determined to have a very high purity with an average composition of CD34+ cells in the range of 96.62% to 97.89%. These results correlate and confirm the purity results determined for the post ISOLEX product from the enumeration assay (displayed above in Table 5). The purity observed on Day 0 was maintained until Day 3 for each of the test formulations at both concentrations. Each of the test formulations contained a small percentage of contaminating cells that were non-specifically carried into the post ISOLEX product. The majority of these cells were B cells, followed in descending order by T cells, granulocytes, and monocytes. The level of CD14/34+ cells in each of the test formulations was low (<0.3%). Analysis of the contaminating cell subsets accounted for approximately 100% of the cells present in each of the test formulations. The percentage of platelets identified in the products averaged to ≦3.2%. In each case, the majority of the platelets identified were co-aggregated with a CD34+ cell (CD61/34+). Overall, the results show that the composition of the cells in each of the test formulations remained very similar despite the solution tested or the time period that the cells were stored.

Clonogenic Potential:

The clonogenic potential of the cells was accessed after storage of the cells at each time point (Days 0 to 3). An aliquot of the cells was collected from each of the test formulations and placed into culture for approximately two weeks. The colonies on each plate were scored for the presence of Colony Forming Unit-Granulocyte Macrophage (CFU-GM), Colony Forming Unit-Erythroid (CFU-E), Burst Forming Unit-Erythroid (BFU-E), and Colony Forming Units with both GM and erythroid colonies (CFU-GEMM). Donor to donor variability was seen in the number of colonies produced. Therefore, the results of this analysis were averaged such that general trends in clonogenic potential correlated to test solutions could be determined.

CFU-GM colonies constituted the greatest number of colonies observed after culture of the cells sampled from each of the test formulations on Day 0. At this time point, the average CFU-GM counts ranged from 93.5 to 109.4. The average number of CFU-GM colonies was best maintained over storage time with the cells stored in PLASMA-LYTE A (Formulations B and C). Under these conditions, a gradual decrease in CFU-GM colony number was observed with an average colony number in the range of 66.4 to 77.5 on Day 3. A larger decrease in the colony number was observed after one day of storage of cells in saline with autologous plasma (Formulation A); similar to that observed after three days of storage of cells in solutions composed of PLASMA-LYTE A (Formulations B and C). The average number of colonies continued to decrease over time and decreased by approximately ≧50% by Day 3.

As was observed with the formation of the CFU-GM colonies, the highest number of BFU-E colonies was maintained for the duration of the storage time with cells stored in PLASMA-LYTE A (Formulations B and C). Culture of these test formulations produced average BFU-E colony numbers in the range of 62.6 to 77.0 on Day 1 with a gradual decrease to a range of 52.8 to 64.3 on Day 3. On Day 1, cells stored in saline with autologous plasma (Formulation A) were found to produce a similar number of BFU-E colonies to cells stored in PLASMA-LYTE A (Formulations B and C), but lower numbers were observed in comparison as the storage time increased. As seen with the formation of CFU-GM colonies, this resulted in a decrease of BFU-E colonies by approximately ≧50% by Day 3.

As is typically observed with CD34+ cells in the CFU assay, only a small number of CFU-E and CFU-GEMM colonies were produced after culture of cells from each of the test formulations after storage for one to three days. Due to the low number of colonies produced, no conclusions can be determined from this analysis.

The acceptance criterion for the CFU assay was pass or fail. Failure of the clonogenic assay is defined as no observable colony formation at the end of the incubation period. This acceptance criterion was met for storage of the cells in each of the test formulations for three days.

The average clonogenic potential (percent clonogenicity) of the cells was calculated for each of the test formulations by totaling each of the cell types observed and dividing by the number of cells originally plated (500 cells/plate). Results indicated that on Day 0, the clonogenic potential of each of the test formulations ranged from 33.6% to 42.3%. The clonogenic potential of each of the test formulations produced very similar results regardless of the starting cell concentration and decreased over storage time. This was most notably observed with cells stored in saline with autologous plasma (Formulation A). Cells stored under this condition had the lowest percent clonogenicity which steadily decreased to an average of ≦18.7% on Day 3. Cells stored in solutions composed of PLASMA-LYTE A (Formulations B and C) retained a higher level of clonogenic potential for a longer period of time. On Day 1, the percent clonogenicity of these test formulations decreased by approximately 2% to 7% in Formulations B and C, respectively. This was maintained until Day 2 and then decreased to levels ≧24.4% on Day 3 in Formulations B and C.

Functionality of Cells:

The functionality of the cells was measured after storage for one to three days in each of the test formulations with assessment of their ability to migrate across a membrane in the presence of increasing concentrations of a chemoattractant, SDF-1α. Donor to donor variability was seen in the functionality of the CD34+ cells in their ability to migrate in response to SDF-1α; therefore, the results of this analysis were averaged such that general trends in migration patterns correlated to test solutions.

Results indicated that on Day 1, cells stored in Formulation A had average migration indexes of 84.8 and 53.2 at starting concentrations of 5.0×10⁶ cells/mL and 7.5×10⁶ cells/mL, respectively. On Day 2 of storage, the average migration indexes decreased to 28.5 and 14.3, respectively. Approximately the same level of migratory capacity that was observed on Day 2 was maintained after three days of storage.

Cells stored in Formulation B displayed a varying range of migratory potential after one day of storage with average migration indexes of 199.1 and 78.8 at starting concentrations of 5.0×10⁶ cells/mL and 7.5×10⁶ cells/mL, respectively. Despite this variability observed between the two concentrations tested, the level of migration observed after storage of the cells in Formulation B was well maintained to indexes of ≧66.3 after storage of the cells for three days. In fact, the level of migration observed with the 7.5×10⁶ cells/mL formulation was nearly identical for the duration of the storage time.

Cells at a starting concentration of 5.0×10⁶ cells/mL stored in Formulation C exhibited a robust level of migration (average migration index of 122.7 on Day 1) that was maintained until Day 3 of storage (average migration index of 100.9). Unlike the data presented thus far, the cells stored in Formulation C at a starting concentration of 7.5×10⁶ cells/mL displayed a variable range of migratory potential that, on average, did not decrease over storage time. It was in fact lowest on Day 1 (average migration index of 54.9), highest on Day 2 (average migration index of 148.6), and then decreased by Day 3 of storage (average migration index of 91.6). Although this is the case, trends in the migratory potential of the cells stored under this condition can be made; such that in general, an average migration index of ≧54.9 was observed.

Overall, a dose dependant migration response was observed for each of the test formulations on each day of analysis. The level of migratory potential observed was fairly similar with each of the test formulations on Day 1. After that time point, the highest levels of migration were noted with cells stored in each of the test solutions composed of Formulations B and C. Cells stored in this manner retained the greatest ability to migrate in a dose dependant manner to increasing concentrations of SDF-1α for up to three days.

The acceptance criterion for the migration assay was pass or fail. Failure of the migration assay is defined as no migration of CD34+ cells in response to SDF-1α resulting in a migration index of 1.0 (same as the control) at each concentration of SDF-1α. Cell migration was measured for each of the test formulations on each day of storage. Therefore, the acceptance criterion for this parameter was achieved for storage of the cells in each of the test formulations for three days. These results indicate that the receptor for SDF-1α (CXCR-4) is maintained to some extent on the cells for a period of at least three days of storage under these conditions. Thus, selected CD34+ cells may be able to home to ischemic areas in response to cytokines after injection of these cells into those sites.

### Conclusion

This study evaluated alternate solutions to saline with autologous plasma (i.e., Formulation A) for storage of CD34+ cells at two concentrations in a syringe at 2-8° C. over a three day period. The goal of the study was to determine under which condition(s) the stability of the cells was maintained. In summary, the following key observations were made during this study:

The viability of the cells stored in each of the formulations at both cell concentrations was maintained (>80%) until Day 3. A significant difference was observed between the viability of the cells stored in saline with autologous plasma (Formulation A) compared to PLASMA-LYTE A with HSA (Formulation C) on Days 1-3 and PLASMA-LYTE A with autologous plasma (Formulation B) on Days 2 and 3. Overall, the highest level of viability was observed with cells stored in PLASMA-LYTE A (i.e., Formulations B and C). The viability of the cells stored in these solutions remained ≧95% until Day 3.

The level of early apoptotic cells was <50% for the cells stored in each of the solutions at both cell concentrations until Day 3. Storage in each of the solutions produced similar results until Day 1. On Days 2 and 3, the most minimal degree of early apoptosis was found with storage of the cells in PLASMA-LYTE A with autologous plasma (Formulation B). This was significantly different than the levels observed with cells stored in saline with autologous plasma (Formulation A) or PLASMA-LYTE A with HSA (Formulation C) at that time.

Cells stored in PLASMA-LYTE A (Formulations B and C) produced the same type of cell differentiation patterns during the three day storage period. The clonogenic potential of the cells stored in both of these solutions was maintained at fairly constant levels from Day 1 to Day 2 (>30%) and then decreased to approximately 24% by Day 3. Cells stored in saline with autologous plasma (Formulation A) had the lowest percent clonogenicity which steadily decreased over time to an average of <19% on Day 3.

A dose dependant cell migration response was measured for each of the test formulations on each day of storage. The ability of the cells to migrate was very similar for each of the test formulations on Day 1. After that time period, the cells stored in PLASMA-LYTE A with autologous plasma (Formulation B) and PLASMA-LYTE A with HSA (Formulation C) retained the highest migratory potential.

Overall, no difference in results was observed due to the concentration of cells utilized in this study. Cells stored in each of the solutions maintained a high degree of viability, low level of apoptosis, and similar levels of clonogenic and migratory potential until Day 1. After that time period, storage of cells in PLASMA-LYTE A with autologous plasma or HSA (Formulations B and C) maintained viability and functionality to the greatest extent. The degree of cells undergoing the early stages of apoptosis was determined to be lowest in cells stored in PLASMA-LYTE A with autologous plasma (Formulation B) at that time. Although this is the case, levels of early apoptotic cells were relatively low overall (<12%) after storage of cells in either solution composed of PLASMA-LYTE A (Formulations B and C).

### Example 3

### Further Experiments Evaluating the Storage and Stability of Compositions Comprising CD34+ Cells (Mobilized from Donors Receiving 5 μg/kg/day G-CSF)

The present Example evaluated the stability of ISOLEX selected CD34+ cells additional experiments after concentration and storage of the CD34+ cells in a syringe in various solutions suitable for injection.

Experimental Design:

Mobilized peripheral blood mononuclear cells (MNCs) were obtained from AllCells (Regimen E, Emeryville, Calif.) by injecting a normal healthy human donor with G-CSF once a day for five consecutive days with a cell collection procedure subsequent to the last dose. The donor received a customized dose of G-CSF per kilogram of body weight per day, or 5 mcg/kg/day. The mobilized MNCs were shipped overnight in temperature-monitored conditions of 1 to 10° C. using 3M TL20 Temperature Loggers (St. Paul, Minn.) and received within 24 hours of collection. Testing on the day of receipt of the apheresis product was referred to as “Day −1” (Day minus 1) because it was the day before the ISOLEX selection and syringe sampling which was referred to as “Day 0” testing. After receipt of the mobilized apheresis unit (Day −1), the product was sampled and analyzed on the Coulter AcT Diff 2 hematology analyzer and a determination of the WBCs (white blood cells), RBCs (red blood cells), and PLT (platelet) concentration was collected. A manual differential was performed to determine the percentage of neutrophils, lymphocytes, monocytes, eosinophils, basophils, and immature cells present in the product. Flow cytometry was utilized to determine the viability and percentage of CD34+ (stem cells), CD3+ (T-cell lymphocytes) and CD19+ (B-cell lymphocytes) cells. After the apheresis product was received into the laboratory and sampled for analysis, the product was stored overnight under refrigerated conditions.

The following day, which is two days post-apheresis collection, the product was sampled again to obtain an automated cell count, manual WBC differential, subset analysis and viability determination. Testing on this day was designated Day 0 because it was the ISOLEX selection day and the initiation of storage of the selected cells into test formulations. The CD34+ cells were selected using the ISOLEX 300i Magnetic Cell Selection Systems (version 2.5) with the positive selection utilizing 9C5 CD34 Monoclonal Antibody and PR34+ Releasing Agent from the ISOLEX Reagent Kit (code #4R9734) and Rat anti-Mouse IgG1 (RAM) paramagnetic beads (Dynabeads® M-450 Cat #428.01D). Samples were collected from the pre- and post-selected product and analyzed for cell counts and CD34+ and CD45+ cell enumeration which determined yield, purity, and viability of the post ISOLEX 300i product. These test parameters were analyzed according to the procedures described above in Example 2.

The CD34+ selected product was concentrated, and re-suspended in the following solutions: saline with 5% autologous plasma (Formulation A), PLASMA-LYTE A with 5% autologous plasma (Formulation B), or PLASMA-LYTE A with 5% HSA (Formulation C) at an approximate concentration of 5×10⁶ cells/mL (±10%) as determined by Coulter cell count. The syringes were loaded into three 1 mL syringes (Becton Dickenson, polycarbonate, luer-lock tip) to a total volume of 2.5 mL and the syringes were packed in Credo Series 4-1296 thermal shipping containers and stored over a time period of one, two or three days (Days 1, 2 and 3) at 1 to 10° C. After three of the ISOLEX 300i selections, it was determined that there were not enough cells available to concentrate and load 2.5 mL of cells into 3 syringes for each of the media conditions. For those experiments, the test formulations were prioritized to ensure testing of saline with 5% autologous plasma (Formulation A) and PLASMA-LYTE A with 5% autologous plasma (Formulation B) over testing of PLASMA-LYTE A with 5% HSA (Formulation C).

Three Crēdo Series 4-1296 thermal shipping containers each containing either Day 1, Day 2 or Day 3 samples and a temperature monitor were shipped via World Courier overnight via NFO (Next Flight Out) and received back into the laboratory the following day (designated Day 1 for in-house testing). The shipping boxes containing the Day 2 and Day 3 samples were stored at room temperature unopened until the appropriate testing day. The Day 1 storage box was opened upon receipt and the conditions were documented. The temperature monitor log was included in the study documentation and the samples were collected from the syringes for analysis. The Day 2 box was opened approximately 2 days after the cell selection procedure and the Day 3 box was opened approximately 3 days after the cell selection procedure.

On each day (Days 1, 2 and 3), cells were collected from the syringes, pooled according to test formulation, and analyzed. Prior to sample collection, the cell solution in the syringe was mixed by rolling the syringe between two hands both vertically and horizontally followed by an end over end mixing technique. Cell viability, apoptosis, and purity (percentage of CD34+ cells and contaminating cell subsets) was determined using procedures described above in Example 2. The functionality and clonogenicity of the CD34+ cells was determined by performing cell migration and CFU (colony forming unit) assays using procedures described above ins Example 2.

Acceptance Criteria was similar to that provided in Example 2.

Data Analysis:

The criterion of 80% for viability and 50% for apoptotic cells was tested as follows. Analysis of variance with repeated measures was used in all analyses. The dependent factors or responses in the analyses were viability and apoptosis respectively. The independent factors in the analysis were storage solutions, number of day stored and the interaction between storage solutions and days of storage. The repeated measures factor was the CD34+ product (ISOLEX Selection Date). Estimates of the Least Square Means were calculated along with 95% confidence intervals of these estimates for each solution and each day of storage. A resultant p-value of less than 0.05 provided evidence that they are not equal. The criteria for the migration and clonogenic assays were met if there were no failures.

Results:

Twelve ISOLEX 300i selections were completed in order to select CD34+ cells for use in this study. In each procedure, a mobilized unit from a single donor was processed on one of two ISOLEX 300i Magnetic cell Selection Systems Instrument A (serial number 3002) and Instrument B (serial number 3210) with RAM beads. Six of the runs selected a sufficient number of cells to enable the analysis of all three test formulations. Three of the runs selected enough cells to enable the analysis of saline with 5% autologous plasma (Formulation A) and PLASMA-LYTE A with 5% autologous plasma (Formulation B), but not PLASMA-LYTE A with 5% HSA (Formulation C).

An additional three runs were terminated at various points after cell selection due to compromised conditions. The first run was terminated after it was discovered that the plasma used to make Formulations A and B was contaminated with bacteria at the time of processing at AllCells. The sixth run was terminated because the ISOLEX 300i selection did not yield a sufficient number of cells for three days of testing with at least two of the test formulations. The tenth run was terminated after it was discovered that the thermal panels in the shipping boxes were not cooled according to the user guide and the cells were inadvertently frozen in the shipping process.

An attempt was made to evenly distribute the cell selections between the two ISOLEX 300i instruments. Of the completed runs, 5 were performed on Instrument A and 4 were performed on Instrument B.

For the purpose of this study, the day of receipt of the apheresis MNC product will be referred to as Day −1 (Day minus 1) and the following day, Day 0. Day −1 is approximately one day post-collection of the apheresis product and Day 0 is approximately two days post-collection of the apheresis product. ISOLEX 300i selection of the CD34+ cells occurred on Day 0. This is also when the test formulations were prepared and testing of the cells in the test formulations began. Tables 11-13 summarize the results of the WBC (white blood cell), RBC (red blood cell), and PLTS (platelet) determination in the apheresis MNC product on Day −1 and Day 0 samples.

TABLE 11
Average WBC Counts × 10⁷ cells/mL

| Run | Day −1 | Day 0 |
|---|---|---|
| 2 | 13.8 | 13.1 |
| 3 | 17.0 | 17.4 |
| 4 | 23.4 | 23.9 |
| 5 | 28.1 | 26.0 |
| 7 | 24.6 | 27.8 |
| 8 | 20.6 | 23.5 |
| 9 | 11.9 | 13.3 |
| 11 | 20.9 | 23.0 |
| 12 | 17.7 | 19.8 |
| Average | 19.8 | 20.9 |
| Std. Dev. | 5.19 | 5.30 |

TABLE 12
Average RBC Counts × 10¹⁰ cells/mL

| Run | Day −1 | Day 0 |
|---|---|---|
| 2 | 0.03 | 0.03 |
| 3 | 0.05 | 0.05 |
| 4 | 0.05 | 0.05 |
| 5 | 0.06 | 0.06 |
| 7 | 0.06 | 0.07 |
| 8 | 0.05 | 0.05 |
| 9 | 0.04 | 0.04 |
| 11 | 0.05 | 0.08 |
| 12 | 0.05 | 0.06 |
| Average | 0.05 | 0.05 |
| Std. Dev. | 0.01 | 0.02 |

TABLE 13
Average PLT Counts × 10⁷ cells/mL

| Run | Day −1 | Day 0 |
|---|---|---|
| 2 | 214 | 205 |
| 3 | 325 | 314 |
| 4 | 287 | 279 |
| 5 | 376 | 366 |
| 7 | 318 | 308 |
| 8 | 294 | 295 |
| 9 | 174 | 184 |
| 11 | 263 | 278 |
| 12 | 333 | 326 |
| Average | 287 | 284 |
| Std. Dev. | 62.3 | 57.4 |

The average WBC, RBC and PLT counts obtained from the Coulter AcT Diff 2 hematology analyzer were comparable from Day −1 to Day 0. These results suggest that storage of the apheresis product overnight at refrigerated conditions does not result in a loss in cell number for the parameters tested.

The percentage of neutrophils, lymphocytes, monocytes and immature cells present in the apheresis MNC product were comparable from Day −1 and Day 0 samples. There is some variability seen with the lymphocytes and monocyte percentages, but the average values from Day −1 to Day 0 are comparable when the standard deviations are taken into consideration. Eosinophil and basophil cells were not visible in any of the samples and therefore, not represented in the tables. The number of immature cells was highly variable from donor to donor, but on average, the Day −1 to Day 0 counts were comparable. The percentages are based on a count of 100 cells. These results suggest that storage of the apheresis product overnight in refrigerated conditions does not result in the loss of the visualized WBC populations visualized.

The average percent viability of the apheresis MNC product was 95.80±5.68 for Day −1 and 93.13±3.15 for Day 0. While the average for the runs suggests that the Day 0 viability is slightly less than the Day −1 viability, the values are comparable when the standard deviations are taken into consideration. These results suggest that storage of the apheresis product overnight in refrigerated conditions does not result in a large decrease in viability.

The percentage of CD34+ stem cells averaged 0.73±0.41 on Day −1 and 0.84±0.39 on Day 0. The CD3+ T-cells averaged 38.74±9.23 on Day −1 and 43.79±11.69 on Day 0. The CD19+ B-Cells averaged 10.72±3.98 on Day −1 and 12.07±4.40 on Day 0. Combined, the percentage of CD34, CD3 and CD19 cells represent 56.70% of the entire apheresis MNC population.

After the ISOLEX 300i selection, samples of the CD34+ cells were analyzed to determine the concentration of WBCs, RBCs, and PLTs present. Using a sample of the pre and post ISOLEX 300i products, the total cells per unit (product loaded onto each ISOLEX instrument) was calculated.

A range of 3.42×10¹⁰ to 7.21×10¹⁰ WBC/unit was loaded onto the ISOLEX 300i device with an average of 5.44×10¹⁰ WBC/unit. This range was below the maximum of 8.0×10¹⁰ cells that represents the maximum number of cells that may be processed per ISOLEX 300i selection². The apheresis units contained an average of 140.57×10⁹ RBCs/unit with a range of 79.56×10⁹ to 207.08×10⁹ for the runs. The platelets averaged 735.84×10⁹ PLTs/unit with a range of 472.99×10⁹ to 967.70×10⁹ per unit for the runs.

Overall the WBC counts of the post ISOLEX 300i products averaged to 2.68×10⁸/unit with a range that encompassed 1.25×10⁸ to 6.77×10⁸WBC/unit. The post ISOLEX 300i products did not contain any measurable levels of RBCs or PLTS based on Coulter cell counts. Runs 2, 3 and 9 had the lowest WBC numbers per apheresis unit of the 9 runs included in this study. Runs 2, 3 and 9 also yielded the lowest post-ISOLEX WBC counts and consequently for these three runs, there were only a sufficient number of cells to test two of the three test formulations.

The total number of CD34+ and CD45+ cells in each of the pre and post samples was determined by flow cytometric analysis. This measurement provided an accurate determination of the total number of these cell types in each pre and post ISOLEX 300i products.

In order to determine the yield of CD34+ cells obtained from each ISOLEX 300i selection, the total number of CD34+ cells in each of the post ISOLEX 300i products was compared to the values obtained for the corresponding pre-ISOLEX samples with the following equation:

$$\% Yield = \frac{Total CD 34 + cells in the post ISOLEX 300 i product}{Total CD 34 + cells in pre ISOLEX 300 i product} \times 100$$

The data was also analyzed to determine the percent purity of each of the post ISOLEX 300i products.

The percent yield of CD34+ cells from the ISOLEX 300i selections averaged 60.42%±1.92% with values ranging between 57.93% and 63.08%. The percent purity of the post ISOLEX product averaged 97.31%±1.26% with values ranging between 95.74% and 99.08%.

After determining the WBC count of the post ISOLEX 300i product, the appropriate number of cells were concentrated and then re-suspended to a target concentration of 5.0×10⁶ cells/mL in each of the following solutions:

Formulation A: Saline with 5% autologous plasma

Formulation B: PLASMA-LYTE A with 5% autologous plasma

Formulation C: PLASMA-LYTE A with 5% HSA

The concentration of each of the test formulations was adjusted to within ±10% of the 5.0×10⁶ cells/mL target concentration and confirmation of the WBC concentration was determined by analysis with the Coulter Act Diff 2 hematology analyzer. The CD34+ cells in the various test solutions were then aspirated using a 16 gauge needle into BD 1 mL syringes with Luer-Lok™ Tip, Ref. 309628, capped with the original needle cap and stored in syringes for up to 3 days in temperature controlled shipping containers at 1 to 10° C. Three syringes with a total of 2.5 mL per test formulation condition per day were prepared. For each test formulation condition, the syringes were prepared such that two syringes contained 1 mL and one syringe contained 0.5 mL.

The Crēdo Series 4-1296 thermal shipping containers from Minnesota Thermal Science were chosen because of their specified thermal performance of holding a temperature range of 1 to 10° C. for 96+ hours. A temperature logger was included in each box and the temperature was recorded for the duration of storage. The syringes were packaged into the shippers by sandwiching three syringes per media condition horizontally between two pieces of non-insulating foam. A temperature logger was placed inside the box between the first and second layers of foam. The shippers traveled by land and air via World Courier using NFO (next flight out) service and were delivered to World Courier, New Hyde Park 11040. The shippers were then returned by land and air to Round Lake 60073 arriving by mid-morning the day after the original shipment. The shippers containing the syringes for Day 1 testing were opened, the temperature logger was retrieved, and the conditions of the syringes were examined. The shippers containing syringes for Day 2 and 3 were stored unopened at room temperature conditions until the appropriate day of testing. All of the shippers (except for Run 10) remained within the temperature range of 1 to 10° C., including the shippers being used for testing on Day 3. Testing for Run 10 was terminated after the cells were frozen in transit due to the pre-conditioning of the Credo thermal panels at −70° C. instead of the user guide recommended −20° C.

The test formulations stored in the syringes were sampled daily (Days 0, 1, 2, and 3) by following a consistent process of rolling each syringe between two hands both vertically and horizontally; 15 times each. This process was repeated and followed by end-over-end mixing five times. The samples from each of the syringes were pooled according to test formulation and analyzed to determine the WBC concentration. Analysis of the WBC concentration was utilized to determine the amount of cells needed for the viability, apoptosis, migration, and CFU assays on each day of analysis and to observe potential changes in cell number over storage time.

The pre-syringe WBC concentrations closely resemble the Day 0 syringe concentrations, which suggest thorough mixing of the test formulations while they were being loaded into the syringes. For each of the test formulations, the WBC concentrations remained within 10% of the Day 0 WBC concentration over the three days of storage. Cells stored in Formulation A exhibited an average decrease in WBC concentration from 4.8×10⁶ cells/mL at Day 0 to 4.4×10⁶ cells/mL at Day 3. Cells stored in Formulation B exhibited an average decrease in WBC concentration from 4.9×10⁶ cells/mL at Day 0 to 4.6×10⁶ cells/mL at Day 3. Cells stored in Formulation C remained stable with an average of 5.1×10⁶ cells/mL at Day 0 and Day 3.

On Days 0, 1 2 and 3, the pooled cell samples were analyzed for viability, apoptosis, purity and clonogenic potential. The functionality of the CD34+ cells was also analyzed on Days 1, 2, and 3 by assessing the ability of the cells to migrate toward the chemoattractant SDF-1α. The data presented herein represents the average values of the results from each of the experiments that were completed with data obtained after storage of cells up to three days.

The viability of the cells was determined with the viability dye, 7-AAD. 7-AAD is a membrane impermeable dye that can only be detected in non-viable cells with compromised membranes. The percentage of total cells that excluded 7-AAD due to the presence of an intact cell membrane was recorded as a percentage of the total population.

The viability of the cells on Day 0 was similar for each of the test formulations with an average of approximately 97%. These results indicate a similar starting condition of the cells before storage for up to three days. Cells stored in PLASMA-LYTE A with autologous plasma and PLASMA-LYTE A with HSA (Test formulations B and C, respectively) maintained high levels of viability for up to three days with an average viability of approximately 94% to 95% at that time. Viability of the cells stored in saline with autologous plasma (Formulation A) started to decline after Day 1 with an average viability of approximately 86% on Day 2 and approximately 79% on Day 3. The primary acceptance criterion for the study is based on a viability of greater than 80%. Based on this criterion, cells stored in Formulation A for three days did not meet the acceptance criteria.

Statistical analysis was also completed on this data set. As defined in the acceptance criteria, if the lower bound of these intervals is greater than 80% then the cells may be stored in that solution at that concentration for the given number of days.

Table 14 provides estimates (Least Squares Means) of the viability of CD34+ cells for product stored from Day 0 through Day 3. It also provides estimates and 95% Confidence Intervals (CI) of the estimates. If the lower bound of the 95% CI is greater than 80% then the storage using a given solution for a given number of days may be considered “Acceptable”. Using this definition, all solutions may be stored for three days except Formulation A, which may only be stored for two days.

TABLE 14
Summary of Viability (% Viable Events, 7-AAD-) Least
Squares Means by Solutions and days of Storage

| | Days of | | 95% CI | | |
|---|---|---|---|---|---|
| Solution | Storage | Estimate | Lower | Upper | Acceptable * |
| Formulation A | 0 | 97.2 | 93.9 | 100.5 | Yes |
| | 1 | 92.9 | 89.6 | 96.1 | Yes |
| | 2 | 86.3 | 83.0 | 89.5 | Yes |
| | 3 | 79.1 | 75.8 | 82.3 | No |
| Formulation B | 0 | 97.3 | 94.0 | 100.5 | Yes |
| | 1 | 95.6 | 92.3 | 98.8 | Yes |
| | 2 | 95.8 | 92.5 | 99.0 | Yes |
| | 3 | 95.1 | 91.8 | 98.3 | Yes |
| Formulation C | 0 | 97.1 | 93.2 | 101.0 | Yes |
| | 1 | 96.3 | 92.4 | 100.1 | Yes |
| | 2 | 95.5 | 91.6 | 99.4 | Yes |
| | 3 | 94.0 | 90.1 | 97.9 | Yes |

* Based on the Lower 95% Confidence Limit on the LS Means > 80%

The differences between the LS Means for each pair of solutions at each day of testing were also calculated. The results are summarized in Table 15. This tests the hypothesis that these differences are equal to zero. A p-value of less than 0.05 provides evidence that they are not equal. There were statistical differences between Formulation A and Formulation B and Formulation C at two and three days of storage. There were no statistical differences between Formulations A-C at two and three days of storage.

TABLE 15
Summary of Viability (% Viable Events, 7-AAD-) Estimates
of Differences Between Least Squares Means

| | Solution 1 - Solution 2 * | | | | | |
|---|---|---|---|---|---|---|
| | | | | Lower | Upper | |
| Days of | Solution | Solution | | 95% | 95% | |
| Storage | 1 | 2 | Difference | CI | CI | p-value * |
| 0 | A | B | −0.0 | −4.1 | 4.0 | 0.981 |
| | | C | 0.1 | −4.5 | 4.8 | 0.950 |
| | B | C | 0.2 | −4.4 | 4.8 | 0.933 |
| 1 | A | B | −2.7 | −6.8 | 1.4 | 0.188 |
| | | C | −3.4 | −8.0 | 1.2 | 0.144 |
| | B | C | −0.7 | −5.3 | 3.9 | 0.766 |
| 2 | A | B | −9.5 | −13.6 | −5.4 | <.001 |
| | | C | −9.2 | −13.8 | −4.6 | <.001 |
| | B | C | 0.2 | −4.4 | 4.8 | 0.917 |
| 3 | A | B | −16.0 | −20.1 | −11.9 | <.001 |
| | | C | −14.9 | −19.5 | −10.3 | <.001 |
| | B | C | 1.1 | −3.5 | 5.7 | 0.631 |

* Tests the hypothesis that the differences are zero

The viability of the cells was further explored by determining the percentage of cells undergoing the various stages of apoptosis. This analysis was completed for the entire cell population present in the post ISOLEX 300i product (regardless of cell surface markers) by staining with Annexin V and PI. Annexin V is a protein with a strong, specific affinity to phosphatidylserine (PS), a component of the phospholipid bilayer of cell membranes. In the early stages of apoptotic cell death, the phospholipid asymmetry of the plasma membrane is disrupted and PS is translocated to the outer layer of the cell plasma membrane. PS exposed on the cell membrane binds Annexin V allowing for the detection of apoptotic cells. At this stage, the cell membrane remains intact. Therefore, staining with Annexin V was completed in conjunction with PI, a membrane impermeable dye, which is similar to 7-AAD, and can only be detected in cells with compromised membranes. This allowed for the identification of early apoptotic cells (Annexin V+/PI−) vs. cells undergoing the latest stages of cell death resulting from either apoptotic or necrotic processes. At this stage, the cell membrane integrity becomes compromised and the cells will stain both with Annexin V and PI.

On Day 0, an average of approximately 2% of the cells in each the test formulations were determined to be in the late stages of apoptosis (or non-viable). The percentage of late apoptotic (or non-viable) cells increased slightly to an average of 3.29% for Formulation B and 3.94% for Formulation C by Day 3 of testing. The percentage of late apoptotic cells in Formulation A increased over the three days of testing to an average of 9.70% by Day 3.

The percentage of early apoptotic cells in each of the test formulations were identified by Annexin V positive and PI negative staining.

On Day 0, the average percentage of cells in the early stages of apoptosis ranged from 2.62% to 3.75% among the three test formulations. After one day of storage, the percentage of early apoptotic cells remained stable for the Formulation B, but the percentage of apoptosis approximately doubled in the cells stored in the saline with Formulation A from 3.59% to 6.63% and Formulation C) (from 2.62% to 6.76%). The cell integrity was most stable overall during storage of the cells in Formulation B with an average of approximately 6% of the cells in the early stages of apoptosis by Day 3 of testing. Analysis of cells stored in Formulation C determined that approximately 12% of cells were in the early stages of apoptosis by Day 3. Cells stored in Formulation A, resulted in greatest increase in early apoptotic cells to cells with an average of approximately 17% by Day 3.

One of the secondary acceptance criteria for determination of the formulation most suitable for storage of the CD34+ cells was the level of apoptosis. Statistical analysis was completed on the early apoptotic data set. Table 15 provides estimates of the Least Squares Means and 95% CI for the early apoptotic cell data stored for 0 to 3 days. According to the defined acceptance criteria, if the upper bound of these intervals is less than 50% then the cells may be stored in that solution at that concentration for the given number of days. Using this definition, the acceptance criterion was met for storage of cells in each of the solutions at both concentrations for up to three days.

TABLE 16
Summary of Apoptosis (% Early Apoptotic Events,
Annexin V+/PI−) Least Squares Means
by Solutions and days of Storage

| | Days of | | 95% CI | | |
|---|---|---|---|---|---|
| Solution | Storage | Estimate | Lower | Upper | Acceptable * |
| Formulation A | 0 | 3.6 | 0.4 | 6.8 | Yes |
| | 1 | 6.6 | 3.5 | 9.8 | Yes |
| | 2 | 10.8 | 7.6 | 14.0 | Yes |
| | 3 | 17.0 | 13.8 | 20.1 | Yes |
| Formulation B | 0 | 3.8 | 0.6 | 6.9 | Yes |
| | 1 | 3.5 | 0.3 | 6.6 | Yes |
| | 2 | 4.4 | 1.3 | 7.6 | Yes |
| | 3 | 6.4 | 3.2 | 9.6 | Yes |
| Formulation C | 0 | 5.5 | 1.9 | 9.2 | Yes |
| | 1 | 6.9 | 3.2 | 10.5 | Yes |
| | 2 | 8.6 | 4.9 | 12.2 | Yes |
| | 3 | 12.0 | 8.3 | 15.7 | Yes |

* Based on the Upper 95% Confidence Limit on the LS Means < 50%

Additional statistical analyses were completed on the apoptosis data set to calculate the differences between the least square means for each pair of solutions at each day of analysis. A summary of the results can be found on Table 17 below.

TABLE 17
Summary of Apoptosis (% Early Apoptotic Events, Annexin V+/PI−))
Estimates of Differences Between Least Squares

| | | | Solution 1-Solution 2* | | | |
|---|---|---|---|---|---|---|
| | | | | Lower | Upper | |
| Days of | Solution | | | 95% | 95% | |
| Storage | 1 | Solution 2 | Difference | CI | CI | p-value* |
| 0 | A | B | −0.2 | −3.7 | 3.3 | 0.926 |
| | | C | −1.9 | −5.9 | 2.0 | 0.330 |
| | B | C | −1.8 | −5.7 | 2.2 | 0.371 |
| 1 | A | B | 3.2 | −0.3 | 6.7 | 0.073 |
| | | C | −0.2 | −4.2 | 3.7 | 0.905 |
| | B | C | −3.4 | −7.4 | 0.5 | 0.088 |
| 2 | A | B | 6.4 | 2.9 | 9.9 | <.001 |
| | | C | 2.3 | −1.7 | 6.2 | 0.254 |
| | B | C | −4.1 | −8.1 | −0.2 | 0.041 |
| 3 | A | B | 10.6 | 7.1 | 14.1 | <.001 |
| | | C | 5.0 | 1.0 | 8.9 | 0.015 |
| | B | C | −5.6 | −9.5 | −1.7 | 0.007 |

*Tests the hypothesis that the differences are zero.

The results of this additional analysis determined that there were statistically significant differences between formulation B and both formulations A and C at 2 and 3 days of storage. There was also a statistical difference between Formulation A and Formulation C at three days of storage.

An extensive subset analysis was completed in order to examine the purity of the samples at each time point. The percentage of the cells positive for CD34 (stem cells), CD3 (T cells), CD19 (B cells), CD14 (monocytes) and CD16 (granulocytes/macrophages) was determined. In addition, cells that were dual positive for CD61/34 (platelet/stem cell co-aggregates) CD14/34 (potential endothelial progenitor cells; EPCs) and CD19/34 (immature B-Cells) was determined. The results are summarized in Table 18 below, for each of the test formulations.

TABLE 18

| | | | | | | | | |
|---|---|---|---|---|---|---|---|---|
| Day | CD34+ | CD3+ | CD19+ | CD14+ | CD16+ | CD61/34+ | CD14/34+ | CD19/34+ |

Formulation A

| | | | | | | | | |
|---|---|---|---|---|---|---|---|---|
| 0 | 97.78 | 0.72 | 1.21 | 0.26 | 0.44 | 1.70 | 0.15 | 0.53 |
| 1 | 98.09 | 0.58 | 0.92 | 0.23 | 0.36 | 1.57 | 0.17 | 0.41 |
| 2 | 98.01 | 0.81 | 0.93 | 0.27 | 0.68 | 2.29 | 0.33 | 0.41 |
| 3 | 97.86 | 1.25 | 0.83 | 0.28 | 0.94 | 2.63 | 0.24 | 0.48 |
| Avg | 97.94 | 0.84 | 0.97 | 0.26 | 0.61 | 2.04 | 0.22 | 0.46 |
| S.D. | 0.14 | 0.29 | 0.16 | 0.02 | 0.26 | 0.50 | 0.08 | 0.06 |

Formulation B

| | | | | | | | | |
|---|---|---|---|---|---|---|---|---|
| 0 | 97.94 | 0.59 | 1.20 | 0.20 | 0.32 | 1.43 | 0.11 | 0.54 |
| 1 | 98.22 | 0.55 | 1.09 | 0.12 | 0.52 | 1.31 | 0.11 | 0.56 |
| 2 | 98.25 | 0.64 | 1.04 | 0.14 | 0.47 | 1.83 | 0.11 | 0.58 |
| 3 | 98.46 | 0.59 | 0.73 | 0.12 | 0.33 | 2.02 | 0.12 | 0.41 |
| Avg | 98.22 | 0.59 | 1.01 | 0.14 | 0.41 | 1.65 | 0.11 | 0.52 |
| S.D. | 0.21 | 0.04 | 0.20 | 0.04 | 0.10 | 0.33 | 0.01 | 0.08 |

Formulation C

| | | | | | | | | |
|---|---|---|---|---|---|---|---|---|
| 0 | 98.01 | 0.90 | 0.92 | 0.25 | 0.72 | 2.47 | 0.10 | 0.50 |
| 1 | 98.53 | 0.66 | 0.79 | 0.10 | 0.59 | 2.45 | 0.15 | 0.50 |
| 2 | 98.74 | 0.81 | 0.61 | 0.08 | 1.00 | 2.24 | 0.13 | 0.44 |
| 3 | 98.94 | 0.69 | 0.54 | 0.22 | 0.37 | 2.08 | 0.29 | 0.51 |
| Avg | 98.55 | 0.76 | 0.71 | 0.16 | 0.67 | 2.31 | 0.17 | 0.49 |
| S.D. | 0.40 | 0.11 | 0.17 | 0.09 | 0.26 | 0.18 | 0.08 | 0.03 |

On Day 0, each of the test formulations was determined to have a very high purity with an average composition of CD34+ cells in the range of 97.94% to 98.55%. These results correlate and confirm the purity results determined for the post ISOLEX product from the enumeration assay. The purity observed on Day 0 was maintained until Day 3 for each of the test formulations. Each of the test formulations contained a small percentage of contaminating cells that were non-specifically carried into the post ISOLEX product. The majority of these cells were B cells, followed in descending order by T cells, granulocytes, and monocytes. The average percent of CD19+ B-Cells was ≦1.01%. The average percent of CD19/34+ Immature B-Cells was ≦0.52%. The level of CD14/34+ cells in each of the test formulations was low (<0.3%). Analysis of the contaminating cell subsets accounted for approximately 100% of the cells present in each of the test formulations. The percentage of platelets (CD61+) co-aggregated to CD34+ cells averaged ≦2.5%. Overall, the results show that the composition of the cells in each of the test formulations remained very similar despite the solution tested or the time period that the cells were stored. The purity of the post Isolex selection as defined by average percentage of CD34+ cells remained very high for all of the testing days and was consistent between the three test formulations. The average purity for all three test formulations over the three days of testing remained approximately 98%.

The percentage of CD19+ B-cells averaged ≦1.21% on Day 0 for all three test formulations. Over the three days of testing, the average percentage of CD19+ B-cells decreased in all three test formulations. The percentage of CD19+ B-cells averaged ≦0.83% on Day 3 for all three test formulations.

Of the CD19+ B-Cells found in the post ISOLEX 300i product, approximately half of those are also CD34+. This suggests that a portion of the CD19+ population is not carried non-specifically into the product, but an active part of the heterogeneous CD34+ population.

The clonogenic potential of the cells was accessed after storage of the cells at each time point (Days 0 to 3). An aliquot of the cells was collected from each of the test formulations and placed into culture for approximately two weeks. The colonies on each plate were scored for the presence of Colony Forming Unit-Granulocyte Macrophage (CFU-GM), Colony Forming Unit-Erythroid (CFU-E), Burst Forming Unit-Erythroid (BFU-E), and Colony Forming Units with both GM and erythroid colonies (CFU-GEMM). Donor to donor variability was seen in the number of colonies produced. Therefore, the results of this analysis were averaged such that general trends in clonogenic potential correlated to test solutions could be determined.

CFU-GM, colony forming units of granulocytes and macrophage colonies, constituted a large portion of the total colonies observed after culture of the cells from each of the test formulations. At this time point, the average CFU-GM counts ranged from 84.9 for Formulation A to 93.5 and 92.4 for Formulations B and C. The average number of CFU-GM colonies was best maintained over storage time with the cells stored in Formulations B and C. Under these conditions, a gradual decrease in CFU-GM colony number was observed with an average colony number in the range of 71.4 to 78.1 on Day 3. The largest decrease in colony number was observed beginning after one day of storage of cells in Formulation A; and continued over the three days of storage. The average number of colonies on Day 3 for formulation A was 26.0 while cells stored in Formulations B and C produced colony counts of 71.4 and 78.1.

BFU-E, burst forming units producing erythroid colonies constituted a portion of the total colonies similar to CFU-GM on Day 0. At this time point, the average BFU-E counts ranged from 83.8 for Formulation A to 91.3 and 93.7 for Formulations B and C, respectively. The average number of BFU-E colonies was best maintained over storage time with the cells stored in Formulations B and C. Under these conditions, the colony counts remained stable through Day 1. On Day 2, a decrease in BFU-E colony number was observed with an average colony number in the range of 72.8 to 76.3 on Day 3. The largest decrease in colony number was observed beginning after one day of storage of cells in saline with autologous plasma (Formulation A); and continued over the three days of storage. The average number of colonies on Day 3 for Formulation A was 43.5 while cells stored in Formulations B and C produced colony counts of 72.8 and 76.3.

As is typically observed with CD34+ cells in the CFU assay, the CFU-GEMM colonies constituted a minority of the colonies produced after culture of cells from each of the test formulations. On Day 0, the average CFU-GEMM counts ranged from 2.9 to 3.3. As was observed with the other colonies, the highest number of CFU-GEMM colonies was maintained for the duration of the storage time with cells stored in Formulations B and C. Culture of Formulations B and C produced average CFU-GEMM colony numbers in the range of 2.4 to 3.6 on Day 1 with a gradual decrease to a range of 1.1 to 1.8 on Day 3. Cells stored in Formulation A produced an average of 3.0 colonies on Day 1 which decrease to 0.8 colonies by Day 3.

Similar to the CFU-GEMM, the CFU-E colonies also typically constitute a minority of the total colonies observed. On Day 0, the average CFU-E colonies ranged from 2.8 to 3.5. As was observed with the other colonies, the highest number of CFU-E colonies was maintained for the duration of the storage time with cells stored in Formulations B and C. Culture of Formulations B and C produced average CFU-E colony numbers of 3.1 to 3.5 on Day 1 with a decrease to an average of 1.5 and 1.7 by Day 3. Cells stored in Formulation A produced an average of 2.8 colonies on Day 1 which decreased to 0.8 colonies by Day 3.

The average clonogenic potential (percent clonogenicity) of the cells was calculated for each of the test formulations by totaling each of the colony types observed and dividing by the number of cells originally plated (500 cells/plate). On Day 0, the clonogenic potential of each of the test formulations ranged from 34.9% to 38.6%. The clonogenic potential of each of the test formulations decreased over storage time. This was most notably observed with cells stored in Formulation A. Cells stored under this condition had the lowest percent clonogenicity which steadily decreased to an average of 14.2% on Day 3. Cells stored in solutions composed of Formulations B and C retained a higher level of clonogenic potential over the three days of storage. The clonogenicity of Formulations B and C remained fairly stable until Day 2 when there was a decrease of approximately 3 percent to 35.0% and 34.8% (Formulations B and C, respectively). By Day 3 the percentage decreased to 29.3% and 31.6% (Formulations B and C, respectively).

The acceptance criterion for the CFU assay was pass or fail. Failure of the clonogenic assay is defined as no observable colony formation at the end of the incubation period. There were no failures for storage of the cells in each of the test formulations for three days. The number and type of colonies produced was similar for Formulations B and C through two days of storage. On the third day of testing, Formulation C produced a slightly higher number of colonies over Formulation B.

Functionality of Cells:

The functionality of the cells was measured after storage for one to three days in each of the solutions with assessment of their ability to migrate across a membrane in the presence of increasing concentrations of the chemoattractant, SDF-1α. Donor to donor variability was seen in the ability of the CD34+ cells to migrate in response to SDF-1α; therefore, the results of this analysis were averaged such that general trends in migration patterns correlated to test solutions.

On Day 1, cells stored in Formulation A had an average migration index of 52.0 with the highest concentration of SDF-1α, 400 ng. On Day 2 of storage, the average migration index decreased to 25.2 and by Day 3 the average migration index decreased even further to 11.7 with 400 ng of SDF-1α.

On Day 1, cells stored in saline Formulation B had an average migration index of 109.1 at the highest concentration of SDF-1α, 400 ng. On Day 2 of storage, the average migration index decreased to 70.1 and by Day 3 the average migration index decreased even further to 34.9 at 400 ng of SDF-1α.

On Day 1, cells stored in Formulation C had an average migration index of 164.2 at the highest concentration of SDF-1α, 400 ng. On Day 2 of storage, the average migration index decreased to 83.5 and by Day 3 the average migration index decreased even further to 39.0 at 400 ng of SDF-1α.

Overall, the CD34+ cells exhibited a dose response to increasing concentrations of SDF-1α with the highest migration index corresponding to the highest dose of SDF-1α, 400 ng. Formulation C had the highest functional response of the three test formulations on Days 1, 2 and 3 of testing. Formulation B had a higher functional response than Formulation A on Days 1, 2 and 3 of testing.

The acceptance criterion for the migration assay was pass or fail. Failure of the migration assay is defined as no migration of CD34+ cells in response to SDF-1α resulting in a migration index of 1.0 (same as the control) at each concentration of SDF-1α. A positive migration index was measured for each of the test formulations on each day of storage. Therefore, the acceptance criterion for this parameter was achieved for storage of the cells in each of the test formulations for three days. These results indicate that the receptor for SDF-1α (CXCR-4) is maintained to some extent on the cells for a period of at least three days of storage under these conditions. Thus, selected CD34+ cells may be able to home to ischemic areas in response to cytokines after injection of these cells into those sites.

### Conclusion

This Example evaluated the stability of selected CD34+ cells (that were mobilized from donors that received 5 μg/kg/day G-CSF for 5 days) after being concentrated in potential injection medias (i.e., Formulations A, B or C), loaded into syringes and shipped in commercially available shipping containers with monitored temperature conditions of 1 to 10° C. daily for three days. The goal of the study was to determine under which condition(s) the stability of the cells was maintained. In summary, the following key observations were made during this study:

1) The primary acceptance criteria for determining if the stability of the cells was maintained was based on viability of the cells with a percentage viability of greater than 80% considered acceptable. Cells stored in Formulations B and C, respectively, maintained high levels of viability for up to three days with an average viability of approximately 94%-95% at Day 3. Cells stored in Formulation A experienced a decline in viability with an average viability of approximately 86% on Day 2 and approximately 79% on Day 3. Based on this criterion, cells stored in Formulation A for three days did not meet the primary acceptance criteria for this study.

2) A statistical evaluation of viability reported that there were statistically significant differences between Formulation A and Formulations B and C at two and three days of storage. There were no statistical differences between Formulation B and Formulation C at two and three days of storage. The statistical evaluation reported that all solutions may be stored for up to three days except for Formulation A which may only be stored for up to two days.

3) The secondary acceptance criteria stating that the level of early apoptotic cells should be less than 50% was met for the cells stored in each of the solutions until Day 3. Storage in each of the solutions produced similar results through Day 1. The cell integrity was most stable overall during storage of the cells in Formulation B an average of approximately 6% of the cells in the early stages of apoptosis by Day 3 of testing. Analysis of cells stored in Formulation C determined that approximately 12% of cells were in the early stages of apoptosis by Day 3. Cells stored in formulation A resulted in greatest increase in early apoptotic cells to cells with an average of approximately 17% by Day 3.

4) A statistical evaluation of early apoptotic cells reported that there were statistically significant differences between formulation B and both Formulation A and Formulation C at two and three days of storage. There was also a statistical difference between Formulation A and formulation C at 3 days of storage.

5) The purity of the post ISOLEX 300i selection as defined by average percentage of CD34+ cells remained very high for all of the testing days and was consistent between the three test formulations. The average purity for all three test formulations over the three days of testing remained approximately 98%. A small percentage of contaminating cells were non-specifically carried into the post ISOLEX product. The majority of these cells were B cells, followed in descending order by T cells, granulocytes, and monocytes.

6) The CFU assay acceptance criterion of colony formation was met for storage of the cells in each of the test formulations for three days. The clonogenicity was similar for Formulations B and C through two days of storage. On the third day of testing, Formulation C produced a slightly higher number of colonies over Formulation B. Cells stored in saline with Formulation A had the lowest level of colony formation, which steadily decreased over the three days of storage.

7) The functional assay acceptance criterion of positive migration was met for storage of the cells in each of the test formulations for three days. In all three test formulations, the CD34+ cells exhibited a dose response to increasing concentrations of SDF-1α with the highest migration index corresponding to the highest dose of SDF-1α, 400 ng. Formulation C had the highest functional response of the three test formulations on Days 1, 2 and 3 of testing. Formulation B displayed a higher functional response than Formulation A on Days 1, 2 and 3 of testing.

Overall, cells stored in Formulations B and C maintained a high level of viability over the three days of testing. Cells stored in Formulation A maintained a viability of greater than 80% for two days of testing. Cells stored in Formulation B maintained the lowest level of apoptosis of the three test formulations and the difference was statistically significant at two and three days of storage. The CD34+ cell purity remained approximately 98% for all three test formulations over the three days of testing. The clonogenicity was similar for cells stored in Formulations B and C through two days of storage. On the third day of testing, cells stored in Formulation C produced a slightly higher number of colonies over those cells stored in Formulation B. Formulation displayed the highest functionality response of the three test formulations on Days 1, 2 and 3 of testing.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Certain embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A pharmaceutical composition comprising (i) a cell population comprising CD34+ cells, (ii) a plasma protein, and (iii) an isotonic solution comprising at least one salt, said isotonic solution comprising acetate, gluconate, or both acetate and gluconate.

2. The pharmaceutical composition of claim 1, wherein the cell population is a heterogeneous cell population of which at least 1% of the cells of the cell population are CD34+ cells.

3.-6. (canceled)

7. The pharmaceutical composition of claim 1, wherein at least 70% of the cells of the cell population are viable cells.

8. The pharmaceutical composition of claim 1, wherein the cell population comprises a subset of cells that express one or more of a cell surface marker selected from the group consisting of CXCR4, c-kit (CD117), FLK-1, (VEGFR-1), Tie-2, KDR (VEGFR-2), CD133, CD45, CD14, CD64, CD61, CD141, CD33, CD38, CD31, CD105, CD146, CD144, CD73, CD99, CD29 and CD90.

9. The pharmaceutical composition of claim 1, wherein at least 1% of cells have a migration index which is greater than that of a negative control, as tested by a chemokine gradient migration assay, after storing the pharmaceutical composition for about 3 to about 5 days at a temperature between 1 and 30 degrees Celsius.

10.-11. (canceled)

12. The pharmaceutical composition of claim 1, wherein the CD34+ cells are CD34+ cells isolated from a human, optionally, wherein the CD34+ cells are CD34+ cells isolated from peripheral blood of the human.

13.-14. (canceled)

15. The pharmaceutical composition of claim 1, wherein the CD34+ cells of the pharmaceutical composition have been formulated with the plasma protein and the isotonic solution for not more than 3 days or not more than 2 days.

16. The pharmaceutical composition of claim 1, wherein the total protein concentration attributed by the plasma protein(s) of the pharmaceutical composition is less than 60 g/L.

17. The pharmaceutical composition of claim 1, comprising no more than five different plasma proteins.

18.-20. (canceled)

21. The pharmaceutical composition of claim 1, comprising albumin.

22.-25. (canceled)

26. The pharmaceutical composition of claim 1, comprising plasma or serum, optionally, wherein the plasma or serum is human plasma or human serum, optionally, wherein the human plasma or human serum is obtained from the human from which the CD34+ cells were isolated.

27.-29. (canceled)

30. The pharmaceutical composition of claim 1, wherein the isotonic solution comprises about 100 mEq to about 180 mEq sodium, (ii) about 1 mEq to about 9 mEq potassium, (iii) about 0.5 mEq to about 5.5 mEq magnesium, (iv) about 70 mEq to about 120 mEq chloride, (v) about 10 mEq to about 40 mEq acetate, and/or (vi) about 10 mEq gluconate to about 40 mEq gluconate.

31.-39. (canceled)

40. The pharmaceutical composition of claim 1, wherein the isotonic solution comprises a crystalloid intravenous fluid comprising electrolytes.

41.-42. (canceled)

43. The pharmaceutical composition of claim 40, wherein the isotonic solution is or is substantially the same as any one of: Plasma-Lyte® A, Plasma-Lyte® 148, Plasma-Lyte® 56, Normosol®-R, Isolyte® P, Lactated Ringer's, Ringer's solution, and 5% Dextrose in Water (D5W).

44.-48. (canceled)

49. A method of repairing tissue damaged by ischemia in a subject, comprising administering to the subject a pharmaceutical composition of claim 1, in an amount effective to repair the tissue in the subject.

50. (canceled)

51. A method of treating a medical condition in a subject in need thereof, comprising administering to the subject a pharmaceutical composition of claim 1, in an amount effective to treat the medical condition in the subject.

52.-56. (canceled)

57. A method of promoting mobilization of CD34+ cells from bone marrow into peripheral blood in a subject, comprising administering to the subject granulocyte colony stimulating factor (G-CSF) at a total administered dose of less than 50 μg/kg.

58.-67. (canceled)

68. A method of obtaining CD34+ cells from a subject, comprising the steps of promoting mobilization of CD34+ cells from bone marrow into peripheral blood in the subject in accordance with the method of claim 57 and collecting CD34+ cells from the peripheral blood of the subject.

69.-72. (canceled)

73. A pharmaceutical composition prepared by (A) administering to a subject granulocyte colony stimulating factor (G-CSF) at a total administered dose of less than 50 μg/kg, (B) collecting CD34+ cells from the peripheral blood of the subject, and (C) formulating the CD34+ cells collected in (B) into a pharmaceutical composition comprising (i) a plasma protein and (ii) an isotonic solution comprising at least one salt, optionally, wherein the isotonic solution comprises acetate, gluconate, or both acetate and gluconate.
